(19) United States
(12) Patent Application Publication
(10) Pub. No.: US 2010/0105714 A1
(43) Pub. Date: Apr. 29, 2010

(54) Furanone Compounds and Methods of Making and Using The Same

(75) Inventors: Lihong Sun, Lexington, MA (US); Wen-Cherng Lee, Lexington, MA (US); Paul Lyne, Arlington, MA (US)

Correspondence Address: Jonathan P. O''Brien, Ph.D.;Honigman Miller Schwartz and Cohn, 350 East Michigan Avenue, Suite 300, KALAMAZOO, MI, 49007, US

(73) Assignee: Biogen Idec MA Inc., Cambridge (US)

(21) Appl. No.: 12/525,144

(22) PCT Filed: Jan. 30, 2008
(86) PCT No.: PCT/US08/01192

§ 371 Date: Oct. 15, 2009

Related U.S. Application Data
(60) Provisional application No. 60/898,293, filed on Jan. 30, 2007.

(51) Int. Cl. A61K 31/438; C07D 307/02; A61K 31/34; C07D 471/10; C07D 471/04; A61K 31/437

(52) U.S. Cl. 514/278; 549/479; 514/473; 546/16; 546/119; 514/303

(57) ABSTRACT

The invention features compounds of the general Formula (I): (formula should be inserted here) Compounds of Formula (I) possess unexpectedly high affinity for Alk5 and/or Alk4, and can be useful as antagonists thereof for preventing and/or treating numerous diseases, including fibrotic disorders.

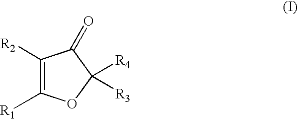

This application claims priority to U.S. Ser. No. 60/898,293, filed on Jan. 30, 2007. The entire contents of the aforementioned application are incorporated herein.

### BACKGROUND OF THE INVENTION

TGFβ (Transforming Growth Factor β) is a member of a large family of dimeric polypeptide growth factors that includes, for example, activins, inhibins, bone morphogenetic proteins (BMPs), growth and differentiation factors (GDFs) and mullerian inhibiting substance (MIS). TGFβ exists in three isoforms (TGFβ1, TGFβ2, and TGFβ3 and is present in most cells, along with its receptors. Each isoform is expressed in both a tissue-specific and developmentally regulated fashion. Each TGFβ isoform is synthesized as a precursor protein that is cleaved intracellularly into a C-terminal region (latency associated peptide (LAP)) and an N-terminal region known as mature or active TGFβ. LAP is typically non-covalently associated with mature TGFβ prior to secretion from the cell. The LAP-TGFβ complex cannot bind to the TGFβ receptors and is not biologically active. TGFβ is generally released (and activated) from the complex by a variety of mechanisms including, for example, interaction with thrombospondin-1 or plasmin.

Following activation, TGFβ binds at high affinity to the type II receptor (TGFβRII), a constitutively active serine/threonine kinase. The ligand-bound type II receptor phosphorylates the TGFβ type I receptor (Alk5) in a glycine/serine rich domain, which allows the type I receptor to recruit and phosphorylate downstream signaling molecules, Smad2 or Smad3. See, e.g., Huse, M. et al., Mol. Cell., 8: 671-682 (2001). Phosphorylated Smad2 or Smad3 can then complex with Smad4, and the entire hetero-Smad complex translocates to the nucleus and regulates transcription of various TGFβ-responsive genes. See, e.g., Massagué, J. Ann. Rev. Biochem. Med., 67: 773 (1998).

Activins are also members of the TGFβ superfamily, which are distinct from TGFβ in that they are homo- or heterodimers of activin βa or βb. Activins signal in a manner similar to TGFβ, that is, by binding to a constitutive serine-threonine receptor kinase, activin type II receptor (ActRIIB), and activating a type I serine-threonine receptor, Alk 4, to phosphorylate Smad2 or Smad3. The consequent formation of a hetero-Smad complex with Smad4 also results in the activin-induced regulation of gene transcription.

Indeed, TGFβ and related factors such as activin regulate a large array of cellular processes, e.g., cell cycle arrest in epithelial and hematopoietic cells, control of mesenchymal cell proliferation and differentiation, inflammatory cell recruitment, immunosuppression, wound healing, and extracellular matrix production. See, e.g., Massagué, J. Ann. Rev Biol., 6: 594-641 (1990); Roberts, A. B. and Sporn, M. B., Peptide Growth Factors and Their Receptors, 95: 419-472, Berlin: Springer-Verlag (1990); Roberts, A. B. and Sporn M. B., Growth Factors 8:1-9 (1993); and Alexandrow, M. G., Moses, H. L. Cancer Res., 55: 1452-1457 (1995). Hyperactivity of TGFβ signaling pathway underlies many human disorders (e.g., excess deposition of extracellular matrix, an abnormally high level of inflammatory responses, fibrotic disorders, and progressive cancers). Similarly, activin signaling and overexpression of activin is linked to pathological disorders that involve extracellular matrix accumulation and fibrosis (see, e.g., Matsuse, T. et al., Am. J. Respir. Cell Mol. Biol. 13: 17-24 (1995); Inoue, S. et al., Biochem. Biophys. Res. Comm. 205: 441-448 (1994); Matsuse, T. et al, Am. J. Pathol. 148: 707-713 (1996); De Bleser et al., Hepatology 26: 905-912 (1997); Pawlowski, J. E., et al., J. Clin. Invest. 100: 639-648 (1997); Sugiyama, M. et al., Gastroenterology 114: 550-558 (1998); Munz, B. et al., EMBO J. 18: 5205-5215 (1999)), inflammatory responses (see, e.g., Rosendahl, A. et al., Am. J. Repir. Cell Mol. Biol. 25: 60-68 (2001)), cachexia or wasting (see Matzuk, M. M. et al., Proc. Nat. Acad. Sci. USA 91: 8817-8821 (1994); Coerver, K. A. et al, Mol. Endocrinol. 10: 534-543 (1996); Cipriano, S. C. et al. Endocrinology 141: 2319-27 (2000)), diseases of or pathological responses in the central nervous system (see Logan, A. et al. Eur. J. Neurosci. 11: 2367-2374 (1999); Logan, A. et al. Exp. Neurol. 159: 504-510 (1999); Masliah, E. et al., Neurochem. Int. 39: 393-400 (2001); De Groot, C. J. A. et al, J. Neuropathol. Exp. Neurol. 58: 174-187 (1999); John, G. R. et al, Nat. Med. 8: 1115-21 (2002)) and hypertension (see Dahly, A. J. et al., Am. J. Physiol. Regul. Integr. Comp. Physiol. 283: R757-67 (2002)). Studies have shown that TGFβ and activin can act synergistically to induce extracellular matrix production (see, e.g., Sugiyama, M. et al., Gastroenterology, 114: 550-558, (1998)). It is therefore desirable to develop modulators (e.g., antagonists) to members of the TGFβ family to prevent and/or treat disorders involving this signaling pathway.

### SUMMARY OF THE INVENTION

The invention is based on the discovery that compounds of Formula (I) are potent antagonists of the TGFβ family type I receptors, Alk5 and/or Alk 4. Thus, compounds of Formula (I) can be employed in the prevention and/or treatment of diseases such as fibrosis (e.g., renal fibrosis, pulmonary fibrosis, and hepatic fibrosis), progressive cancers, or other diseases for which reduction of TGFβ family signaling activity is desirable.

In one aspect, the present invention provides compounds of Formula (I)

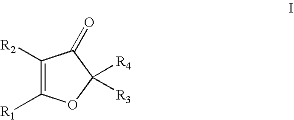

an N-oxide derivative, or a pharmaceutically acceptable salt thereof. Referring to Formula (I),

R¹ is an optionally substituted aryl or an optionally substituted heteroaryl;

R² is an optionally substituted aryl or an optionally substituted heteroaryl; and

Each of R³ and R⁴ is independently an optionally substituted aliphatic, an optionally substituted aryl, or an optionally substituted heteroaryl; or R³ and R⁴, together with the atom to which they are attached, form an optionally substituted 5- to 8-membered cycloaliphatic or an optionally substituted 5- to 8-membered heterocycloaliphatic ring. Unless otherwise specified, each of the ring systems for R₁, R₂, R₃, and R₄ recited herein can be linked to an adjacscent moiety at any position of the ring system.

In some embodiments, R¹ is an optionally substituted aryl.

In some embodiments, R¹ is phenyl optionally substituted with 1 to 3 substituents each independently selected from the group consisting of alkyl, alkenyl, alkynyl, alkoxy, acyl, halo, hydroxy, amino, nitro, cyano, guanadino, amidino, carboxy, amido, alkoxycarbonyl, alkylcarbonyloxy, urea, thiourea, carbamoyl, cycloalkyl, cycloalkyloxy, cycloalkylcarbonyl, heterocycloalkyl, heterocycloalkyloxy, heterocycloalkylcarbonyl, aryl, aryloxy, arylsulfanyl, aroyl, heteroaryl, heteroaryloxy, and heteroaroyl.

In some embodiments, R¹ is an optionally substituted monocyclic heteroaryl.

In some embodiments, R¹ is optionally substituted pyridyl or pyrimidinyl.

In some embodiments, R¹ is an optionally substituted bicyclic heteroaryl. Examples of optionally substituted bicyclic heteroaryl include, but are not limited to, benzo[1,3]dioxolyl, benzo[b]thiophenyl, benzooxadiazolyl, benzothiadiazolyl, benzoimidazolyl, benzooxazolyl, benzothiazolyl, 2-oxo-benzooxazolyl, 2,3-dihydrobenzo[1,4]dioxyl, 2,3-dihydrobenzofuryl, 2,3-dihydrobenzo[b]thiophenyl, 3,4-dihydrobenzo[1,4]oxazinyl, 3-oxo-benzo[1,4]oxazinyl, 1,1-dioxo-2,3 dihydrobenzo[b]thiophenyl, [1,2,4]triazolo[1,5 a]pyridyl, [1,2,4]triazolo[4,3 a]pyridyl, quinolinyl, quinoxalinyl, quinazolinyl, isoquinolinyl, or cinnolinyl; and is optionally substituted.

In some embodiments, R¹ is benzo[1,3]dioxolyl.

In some embodiments, R¹ is optionally substituted [1,2,4]triazolo[1,5-a]pyridin-6-yl.

In some embodiments, R¹ is optionally substituted quinoxalin-6-yl.

In some embodiments, R¹ is substituted with 1 to 3 substituents each independently selected from the group consisting of alkyl, alkenyl, alkynyl, alkoxy, acyl, halo, hydroxy, amino, nitro, oxo, thioxo, cyano, guanadino, amidino, carboxy, sulfo, mercapto, alkylsulfanyl, alkylsulfinyl, alkylsulfonyl, amido, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, cycloalkylcarbonyl, heterocycloalkyl, heterocycloalkyloxy, heterocycloalkylsulfanyl, heterocycloalkylcarbonyl, aryl, aryloxy, arylsulfanyl, aroyl, heteroaryl, heteroaryloxy, heteroarylsulfanyl, and heteroaroyl.

In some embodiments, R² is an optionally substituted aryl, e.g., optionally substituted phenyl.

In some embodiments, R² is phenyl substituted with 1 to 3 substituents each independently selected from the group consisting of alkyl, alkenyl, alkynyl, alkoxy, acyl, halo, hydroxy, amino, nitro, oxo, thioxo, cyano, guanadino, amidino, carboxy, sulfo, mercapto, alkylsulfanyl, alkylsulfinyl, alkylsulfonyl, amido, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, cycloalkylcarbonyl, heterocycloalkyl, heterocycloalkyloxy, heterocycloalkylsulfanyl, heterocycloalkylcarbonyl, aryl, aryloxy, arylsulfanyl, aroyl, heteroaryl, heteroaryloxy, heteroarylsulfanyl, and heteroaroyl.

In some embodiments, R² is o-, m-, or p-methylphenyl.

In some embodiments, R² is an optionally substituted heteroaryl.

In some embodiments, R² is an optionally substituted monocyclic heteroaryl.

In some embodiments, R² is optionally substituted pyridyl or optionally substituted pyrimidinyl.

In some embodiments, R² is an optionally substituted bicyclic heteroaryl. Examples of such bicyclic heteroaryl include, but are not limited to, benzo[1,3]dioxolyl, benzo[b]thiophenyl, benzooxadiazolyl, benzothiadiazolyl, benzoimidazolyl, benzooxazolyl, benzothiazolyl, 2-oxo-benzooxazolyl, 2,3-dihydrobenzo[1,4]dioxyl, 2,3-dihydrobenzofuryl, 2,3-dihydrobenzo[b]thiophenyl, 3,4-dihydrobenzo[1,4]oxazinyl, 3-oxo-benzo[1,4]oxazinyl, 1,1-dioxo-2,3-dihydrobenzo[b]thiophenyl, [1,2,4]triazolo[1,5 a]pyridyl, [1,2,4]triazolo[4,3 a]pyridyl, quinolinyl, quinoxalinyl, quinazolinyl, isoquinolinyl, and cinnolinyl.

In some embodiments, R² is optionally substituted benzo[1,3]dioxolyl.

In some embodiments, R² is substituted with 1 to 3 substituents each independently selected from the group consisting of alkyl, alkenyl, alkynyl, alkoxy, acyl, halo, hydroxy, amino, nitro, oxo, thioxo, cyano, guanadino, amidino, carboxy, sulfo, mercapto, alkylsulfanyl, alkylsulfinyl, alkylsulfonyl, amido, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, cycloalkylcarbonyl, heterocycloalkyl, heterocycloalkyloxy, heterocycloalkylsulfanyl, heterocycloalkylcarbonyl, aryl, aryloxy, arylsulfanyl, aroyl, heteroaryl, heteroaryloxy, heteroarylsulfanyl, and heteroaroyl.

In some embodiments, each of R³ and R⁴ is independently an optionally substituted C₁₋₆ alkyl.

In some embodiments, R³ and R⁴, together with the atom to which they are attached, form a 5- to 8-membered optionally substituted cycloaliphatic or an optionally substituted 5- to 8-membered heterocycloaliphatic ring.

In some embodiments, R³ and R⁴ together with the atom to which they are attached form an optionally substituted 5- to 8-membered cycloaliphatic ring compound of Formula (Ia).

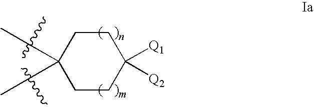

Referring to Formula (Ia),

each of m and n is independently 0, 1, 2, 3 or 4, provided that the sum of m and n is 1, 2, 3, 4 or 5; and

each of Q₁ and Q₂ is independently H, alkyl, alkenyl, alkynyl, alkoxy, acyl, halo, hydroxy, amino, azido, nitro, cyano, guanadino, amidino, carboxy, sulfo, mercapto, alkylsulfanyl, alkylsulfinyl, alkylsulfonyl, arylsulfonyl, amido, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, cycloalkylcarbonyl, heterocycloalkyl, heterocycloalkyloxy, heterocycloalkylsulfanyl, heterocycloalkylcarbonyl, aryl, aryloxy, arylsulfanyl, aroyl, heteroaryl, heteroaryloxy, heteroarylsulfanyl, or heteroaroyl; or

Q₁ and Q₂, together with the atom to which they are attached, form oxo (i.e., ═O), optionally substituted imino (i.e., ═N—R), or optionally substituted alkene (i.e., ═CRR′); or Q₁ and Q₂, together with the atom to which they are attached, form an optionally substituted 5- to 7-membered cycloaliphatic or an optionally substituted 5- to 7-membered heterocycloaliphatic ring.

In some embodiments, Q₁ is H; and Q₂ is hydroxy, alkoxy, alkylcarbonyloxy, or carbamoyl.

In some embodiments, Q₁ is H; and Q₂ is amino, azido, alkylsulfonylamino, arylsulfonylamino, alkylamido, arylamido, heteroarylamido, urea or aminosulfonylamino.

In some embodiments, Q₁ is H; and Q₂ is alkoxycarbonyl substituted aliphatic, carboxy substituted aliphatic, or amido substituted aliphatic.

In some embodiments, Q₁ and Q₂, together with the atom to which they are attached, form oxo or optionally substituted imino.

In some embodiments, Q₁ and Q₂, together with the atom to which they are attached, form a 5- to 7-membered cycloaliphatic or a 5- to 7-membered heterocycloaliphatic ring.

In some embodiments, R³ and R⁴, together with the atom to which they are attached, form an optionally substituted 5- to 8-membered heterocycloaliphatic ring of Formula (Ib),

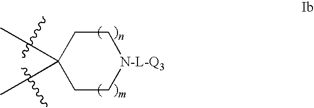

### Referring to Formula (Ib),

each of m and n is independently 0, 1, 2, 3 or 4, provided that the sum of m and n is 1, 2, 3, 4 or 5;

L is a bond, C(O) or S(O)ₚ;

p is 0, 1 or 2; and

Q₃ is H, optionally substituted aliphatic, optionally substituted aryl, optionally substituted cycloaliphatic, optionally substituted heterocycloaliphatic, amino, amido optionally substituted alkoxy, or optionally substituted aryloxy.

In some embodiments, L-Q₃ is H, alkoxycarbonyl, or amido.

In some embodiments, L-Q₃ is acyl, aroyl, alkylsulfonyl, or arylsulfonyl.

In some embodiments, each of m and n is independently 1.

In some embodiments, the compound of Formula (I) is
tert-butyl 3-(benzo[d][1,3]dioxol-5-yl)-4-oxo-2-(pyridin-4-yl)-1-oxa-8-azaspiro[4.5]dec-2-ene-8-carboxylate;
benzyl 3-(benzo[d][1,3]dioxol-5-yl)-4-oxo-2-(pyridin-4-yl)-1-oxa-8-azaspiro[4.5]dec-2-ene-8-carboxylate;
3-(benzo[d][1,3]dioxol-5-yl)-2-(pyridin-4-yl)-1-oxa-8-azaspiro[4.5]dec-2-en-4-one;
4-(3-(benzo[d][1,3]dioxol-5-yl)-4-oxo-2-(pyridin-4-yl)-1-oxa-8-azaspiro[4.5]dec-2-enecarbonyl)benzonitrile;
tert-butyl 2-(benzo[d][1,3]dioxol-5-yl)-4-oxo-3-(pyridin-3-yl)-1-oxa-8-azaspiro[4.5]dec-2-ene-8-carboxylate;
tert-butyl 2-(benzo[d][1,3]dioxol-5-yl)-4-oxo-3-(pyridin-4-yl)-1-oxa-8-azaspiro[4.5]dec-2-ene-8-carboxylate;
2-(benzo[d][1,3]dioxol-5-yl)-3-(pyridin-3-yl)-1-oxa-8-azaspiro[4.5]dec-2-en-4-one;
benzyl 2-(benzo[d][1,3]dioxol-5-yl)-4-oxo-3-(pyridin-4-yl)-1-oxa-8-azaspiro[4.5]dec-2-ene-8-carboxylate;
benzyl 2-(benzo[d][1,3]dioxol-5-yl)-4-oxo-3-(pyridin-3-yl)-1-oxa-8-azaspiro[4.5]dec-2-ene-8-carboxylate;
2-(benzo[d][1,3]dioxol-5-yl)-8-(4-hydroxybenzoyl)-3-(pyridin-3-yl)-1-oxa-8-azaspiro[4.5]dec-2-en-4-one;
2-(benzo[d][1,3]dioxol-5-yl)-3-(pyridin-3-yl)-8-tosyl-1-oxa-8-azaspiro[4.5]dec-2-en-4-one;
tert-butyl 3-(benzo[d][1,3]dioxol-5-yl)-4-oxo-2-(pyridin-2-yl)-1-oxa-8-azaspiro[4.5]dec-2-ene-8-carboxylate;
3-(benzo[d][1,3]dioxol-5-yl)-2-(pyridin-2-yl)-1-oxa-8-azaspiro[4.5]dec-2-en-4-one;
benzyl 3-(benzo[d][1,3]dioxol-5-yl)-4-oxo-2-(pyridin-2-yl)-1-oxa-8-azaspiro[4.5]dec-2-ene-8-carboxylate;
4-(3-(benzo[d][1,3]dioxol-5-yl)-4-oxo-2-(pyridin-2-yl)-1-oxa-8-azaspiro[4.5]dec-2-enecarbonyl)benzonitrile;
3-(benzo[d][1,3]dioxol-5-yl)-2-(pyridin-2-yl)-8-tosyl-1-oxa-8-azaspiro[4.5]dec-2-en-4-one;
3-(benzo[d][1,3]dioxol-5-yl)-N-benzyl-4-oxo-2-(pyridin-2-yl)-1-oxa-8-azaspiro[4.5]dec-2-ene-8-carboxamide;
tert-butyl 2-(benzo[d][1,3]dioxol-5-yl)-4-oxo-3-(pyridin-2-yl)-1-oxa-8-azaspiro[4.5]dec-2-ene-8-carboxylate;
2-(benzo[d][1,3]dioxol-5-yl)-3-(pyridin-2-yl)-8-tosyl-1-oxa-8-azaspiro[4.5]dec-2-en-4-one;
2-(benzo[d][1,3]dioxol-5-yl)-8-(4-chlorophenylsulfonyl)-3-(pyridin-2-yl)-1-oxa-8-azaspiro[4.5]dec-2-en-4-one;
2-(benzo[d][1,3]dioxol-5-yl)-8-(3,4-dichlorophenylsulfonyl)-3-(pyridin-2-yl)-1-oxa-8-azaspiro[4.5]dec-2-en-4-one;
4-(2-(benzo[d][1,3]dioxol-5-yl)-4-oxo-3-(pyridin-2-yl)-1-oxa-8-azaspiro[4.5]dec-2-en-8-ylsulfonyl)benzoic acid;
4-(2-(benzo[d][1,3]dioxol-5-yl)-4-oxo-3-(pyridin-2-yl)-1-oxa-8-azaspiro[4.5]dec-2-en-8-ylsulfonyl)benzonitrile;
3-(benzo[d][1,3]dioxol-5-yl)-8-(4-chlorophenylsulfonyl)-2-(pyridin-2-yl)-1-oxa-8-azaspiro[4.5]dec-2-en-4-one;
3-(benzo[d][1,3]dioxol-5-yl)-8-(3,4-dichlorophenylsulfonyl)-2-(pyridin-2-yl)-1-oxa-8-azaspiro[4.5]dec-2-en-4-one;
4-(3-(benzo[d][1,3]dioxol-5-yl)-4-oxo-2-(pyridin-2-yl)-1-oxa-8-azaspiro[4.5]dec-2-en-8-ylsulfonyl)benzoic acid;
4-(3-(benzo[d][1,3]dioxol-5-yl)-4-oxo-2-(pyridin-2-yl)-1-oxa-8-azaspiro[4.5]dec-2-en-8-ylsulfonyl)benzonitrile;
2-([1,2,4]triazolo[1,5-a]pyridin-6-yl)-3-m-tolyl-1-oxaspiro[4.5]dec-2-ene-4,8-dione;
10-[1,2,4]Triazolo[1,5-a]pyridin-6-yl-11-(6-methylpyridine-2-yl)-1,4,9-trioxa-dispiro[4.2.4.2]tetradec-10-en-12-one;
10-(quinoxalin-6-yl)-11-(3-methylphenyl)-1,4,9-tri oxa-dispiro[4.2.4.2]tetradec-10-en-12-one;
2-(quinoxalin-6-yl)-3-m-tolyl-1-oxaspiro[4.5]dec-2-ene-4,8-dione;
10-(quinoxalin-6-yl)-11-(3-chlorophenyl)-1,4,9-trioxa-dispiro[4.2.4.2]tetradec-10-en-12-one;
10-[1,2,4]Triazolo[1,5-a]pyridin-6-yl-11-(3-m-tolyl)-1,4,9-trioxa-dispiro[4.2.4.2]tetradec-10-en-12-one;
2-([1,2,4]triazolo[1,5-a]pyridin-6-yl)-8-hydroxy-3-m-tolyl-1-oxaspiro[4.5]dec-2-en-4-one;
3-(3-chlorophenyl)-2-(quinoxalin-6-yl)-1-oxaspiro[4.5]dec-2-ene-4,8-dione;
3-(3-chlorophenyl)-8-hydroxy-2-(quinoxalin-6-yl)-1-oxaspiro[4.5]dec-2-en-4-one;
8-hydroxy-2-(quinoxalin-6-yl)-3-m-tolyl-1-oxaspiro[4.5]dec-2-en-4-one;
ethyl 2-(4-oxo-2-(quinoxalin-6-yl)-3-m-tolyl-1-oxaspiro[4.5]dec-2-en-8-ylidene)acetate;
2-(4-oxo-2-(quinoxalin-6-yl)-3-m-tolyl-1-oxaspiro[4.5]dec-2-en-8-ylidene)acetic acid;
2-(4-oxo-2-(quinoxalin-6-yl)-3-m-tolyl-1-oxaspiro[4.5]dec-2-en-8-yl)acetic acid;
N-(2-morpholinoethyl)-2-(4-oxo-2-(quinoxalin-6-yl)-3-m-tolyl-1-oxaspiro[4.5]dec-2-en-8-yl)acetamide;
N-(2-(dimethylamino)ethyl)-2-(4-oxo-2-(quinoxalin-6-yl)-3-m-tolyl-1-oxaspiro[4.5]dec-2-en-8-yl)acetamide;
ethyl 2-(3-(3-chlorophenyl)-4-oxo-2-(quinoxalin-6-yl)-1-oxaspiro[4.5]dec-2-en-8-ylidene)acetate;
2-(3-(3-chlorophenyl)-4-oxo-2-(quinoxalin-6-yl)-1-oxaspiro[4.5]dec-2-en-8-yl)acetic acid;
2-([1,2,4]triazolo[1,5-a]pyridin-6-yl)-8-azido-3-m-tolyl-1-oxaspiro[4.5]dec-2-en-4-one;
2-([1,2,4]triazolo[1,5-a]pyridin-6-yl)-8-amino-3-m-tolyl-1-oxaspiro[4.5]dec-2-en-4-one;
N-(2-([1,2,4]triazolo[1,5-a]pyridin-6-yl)-4-oxo-3-m-tolyl-1-oxaspiro[4.5]dec-2-en-8-yl)methanesulfonamide;
N-methyl-2-(4-oxo-2-(quinoxalin-6-yl)-3-m-tolyl-1-oxaspiro[4.5]dec-2-en-8-yl)acetamide;
N-(2-methoxyethyl)-2-(4-oxo-2-(quinoxalin-6-yl)-3-m-tolyl-1-oxaspiro[4.5]dec-2-en-8-yl)acetamide;
2-(3-(3-chlorophenyl)-4-oxo-2-(quinoxalin-6-yl)-1-oxaspiro[4.5]dec-2-en-8-yl)-N-(2-morpholinoethyl)acetamide;
N-(2-([1,2,4]triazolo[1,5-a]pyridin-6-yl)-4-oxo-3-m-tolyl-1-oxaspiro[4.5]dec-2-en-8-yl)acetamide;
1-(2-([1,2,4]triazolo[1,5-a]pyridin-6-yl)-4-oxo-3-m-tolyl-1-oxaspiro[4.5]dec-2-en-8-yl)urea;
8-aminosulfonylamino-2-([1,2,4]triazolo[1,5-a]pyridin-6-yl)-3-m-tolyl-1-oxaspiro[4.5]dec-2-en-4-one;
5-([1,2,4]triazolo[1,5-a]pyridin-6-yl)-2,2-dimethyl-4-m-tolylfuran-3(2H)-one;
2-([1,2,4]triazolo[1,5-a]pyridin-6-yl)-8-hydroxy-3-(6-methylpyridin-2-yl)-1-oxaspiro[4.5]dec-2-en-4-one;
2-(4-oxo-2-(quinoxalin-6-yl)-3-m-tolyl-1-oxaspiro[4.5]dec-2-en-8-yl)acetamide;
Ethyl 2-(2-([1,2,4]triazolo[1,5-a]pyridin-6-yl)-4-oxo-3-m-tolyl-1-oxaspiro[4.5]dec-2-en-8-ylidene)acetate;
2-(2-([1,2,4]triazolo[1,5-a]pyridin-6-yl)-4-oxo-3-m-tolyl-1-oxaspiro[4.5]dec-2-en-8-ylamino)-2-oxoethyl acetate;
methyl 3-(2-([1,2,4]triazolo[1,5-a]pyridin-6-yl)-4-oxo-3-m-tolyl-1-oxaspiro[4.5]dec-2-en-8-ylamino)-3-oxopropanoate;
dimethyl 3,3′-(2-([1,2,4]triazolo[1,5-a]pyridin-6-yl)-4-oxo-3-m-tolyl-1-oxaspiro[4.5]dec-2-en-8-ylazanediyl)bis(3-oxopropanoate);
3-(2-([1,2,4]triazolo[1,5-a]pyridin-6-yl)-4-oxo-3-m-tolyl-1-oxaspiro[4.5]dec-2-en-8-ylamino)-3-oxopropanoic acid;
N-hydroxy-2-(4-oxo-2-(quinoxalin-6-yl)-3-m-tolyl-1-oxaspiro[4.5]dec-2-en-8-yl)acetamide;
ethyl 2-(2-([1,2,4]triazolo[1,5-a]pyridin-6-yl)-4-oxo-3-m-tolyl-1-oxaspiro[4.5]dec-2-en-8-yl)acetate (isomer A);
ethyl 2-(2-([1,2,4]triazolo[1,5-a]pyridin-6-yl)-4-oxo-3-m-tolyl-1-oxaspiro[4.5]dec-2-en-8-yl)acetate (isomer B);
2-(2-([1,2,4]triazolo[1,5-a]pyridin-6-yl)-4-oxo-3-m-tolyl-1-oxaspiro[4.5]dec-2-en-8-ylidene)acetamide;
2-(2-([1,2,4]triazolo[1,5-a]pyridin-6-yl)-4-oxo-3-m-tolyl-1-oxaspiro[4.5]dec-2-en-8-ylidene)-N-(pyridin-3-yl)methyl)acetamide;
2-(2-([1,2,4]triazolo[1,5-a]pyridin-6-yl)-4-oxo-3-m-tolyl-1-oxaspiro[4.5]dec-2-en-8-ylidene)-N-(3-(2-oxopyrrolidin-1-yl)propyl)acetamide;
11-(3-chloro-4-fluoro-phenyl)-10-[1,2,4]triazolo[1,5-a]pyridin-6-yl-1,4,9-trioxa-dispiro[4.2.4.2]tetradec-10-en-12-one;
3-(3-chloro-4-fluoro-phenyl)-2-[1,2,4]triazolo[1,5-a]pyridin-6-yl-1-oxa-spiro[4.5]dec-2-ene-4,8-dione;
ethyl 2-(2-([1,2,4]triazolo[1,5-a]pyridin-6-yl)-3-(3-chloro-4-fluorophenyl)-4-oxo-1-oxaspiro[4.5]dec-2-en-8-ylidene)acetate;
2-([1,2,4]triazolo[1,5-a]pyridin-6-yl)-8-(1,3-dioxolan-2-yl)-3-m-tolyl-1-oxaspiro[4.5]dec-2-en-4-one;
5-([1,2,4]triazolo[1,5-a]pyridin-6-yl)-4-(3-chlorophenyl)-2,2-dimethylfuran-3(2H)-one;
5-([1,2,4]triazolo[1,5-a]pyridin-6-yl)-4-(3-chloro-4-fluorophenyl)-2,2-dimethylfuran-3(2H)-one;
2-([1,2,4]triazolo[1,5-a]pyridin-6-yl)-8-((pyridin-3-ylamino)methyl)-3-m-tolyl-1-oxaspiro[4.5]dec-2-en-4-one;
2-([1,2,4]triazolo[1,5-a]pyridin-6-yl)-8-(1,3-dioxolan-2-yl)-3-(4-fluoro-3-methylphenyl)-1-oxaspiro[4.5]dec-2-en-4-one;
2-([1,2,4]triazolo[1,5-a]pyridin-6-yl)-3-(4-fluoro-3-methylphenyl)-4-oxo-1-oxaspiro[4.5]dec-2-ene-8-carboxylic acid; or
2-([1,2,4]triazolo[1,5-a]pyridin-6-yl)-3-(4-fluoro-3-methylphenyl)-4-oxo-1-oxaspiro[4.5]dec-2-ene-8-carboxamide.

In another aspect, the invention pertains to a pharmaceutical composition which includes a compound described above and a pharmaceutically acceptable carrier.

In another aspect, the invention provides a method of inhibiting the TGFβ signaling pathway in a subject (e.g., a mammal such as human). The method includes administering to the subject in need thereof an effective amount of at least one of the compounds described above.

In another aspect, the invention provides a method of inhibiting the TGFβ type I receptor in a cell, wherein the method includes contacting the cell with an effective amount of at least one of the compounds described above.

In another aspect, the invention provides a method of reducing the accumulation of excess extracellular matrix induced by TGFβ in a subject, wherein the method includes administering to the subject in need thereof an effective amount of at least one of the compounds described above.

In another aspect, the invention provides a method of treating or preventing a fibrotic condition in a subject, wherein the method includes administering to the subject in need thereof an effective amount of at least one of the compounds described above. Examples of the fibrotic condition include, but are not limited to, scleroderma, lupus nephritis, connective tissue disease, wound healing, surgical scarring, spinal cord injury, CNS scarring, acute lung injury, idiopathic pulmonary fibrosis, radiation-induced pulmonary fibrosis, chronic obstructive pulmonary disease, adult respiratory distress syndrome, acute lung injury, drug-induced lung injury, glomerulonephritis, diabetic nephropathy, hypertension-induced nephropathy, alimentary track or gastrointestinal fibrosis, renal fibrosis, hepatic or biliary fibrosis, liver cirrhosis, primary biliary cirrhosis, fatty liver disease, primary sclerosing cholangitis, restenosis (e.g., coronary restenosis, peripheral restenosis, or carotid restenosis), radiation-induced fibrosis, chemotherapy-induced fibrosis, cardiac fibrosis, opthalmic scarring, fibrosclerosis, a fibrotic cancer, a fibroid, fibroma, a fibroadenoma, a fibrosarcoma, transplant arteriopathy, mesothelioma, and keloid. The fibrotic condition can be idiopathic in nature, genetically linked, or induced by radiation.

In another aspect, the invention provides a method of treating or preventing vascular disease or hypertension in a subject, wherein the method includes administering to a subject in need thereof an effective amount of at least one of the compounds described above. Examples of the vascular disease include, but are not limited to, intimal thickening, vascular remodeling, and organ transplant-related vascular disease; and the example of the hypertension include, but are not limited to, primary or secondary hypertension, systolic hypertension, pulmonary hypertension, or hypertension-induced vascular remodeling.

In some embodiments of each of the methods described above, the compound use is administered locally or via an implantable device (e.g., a delivery pump or a stent).

In another aspect, the invention provides a method of inhibiting growth or metastasis of tumor cells or cancer in a subject, wherein the method include administering to the subject need thereof an effective amount of at least one of the compounds described above.

In another aspect, the invention provides a method of treating a disease or disorder mediated by an overexpression of TGFβ in a subject, wherein the method includes administering to the subject in need thereof an effective amount of at least one of the compounds described above. In some embodiments, the carcinoma is mediated by overexpression of TGFβ (e.g., the carcinoma of the lung, breast, liver, biliary tract, gastrointestinal tract, head and neck, pancreas, prostate, cervix, multiple myeloma, melanoma, glioma, or glioblastomas). In some other embodiments, the disease or disorder is selected from the group consisting of demyelination of neurons in multiple sclerosis, Alzheimer's disease, cerebral angiopathy, squamous cell carcinomas, multiple myeloma, melanoma, glioma, glioblastomas, leukemia, sarcomas, leiomyomas, mesothelioma, and carcinomas of the lung, breast, ovary, cervix, liver, biliary tract, gastrointestinal tract, pancreas, prostate, head, and neck.

In some embodiments of this invention, any of the methods described above can further include administering another active agent (e.g., anticancer or antimicrobial agent), either separately or together with the compound of Formula (I).

An N-oxide (also known as amino oxide) derivative or a pharmaceutically acceptable salt of each of the compounds of Formula (I) is also within the scope of this invention. For example, a nitrogen-containing heterocyclyl substituent can form an oxide in the presence of a suitable oxidizing agent such as m-chloroperbenzoic acid or H₂O₂.

A compound of Formula (I) that is acidic in nature (e.g., having a carboxyl or phenolic hydroxyl group) can form a pharmaceutically acceptable salt such as a sodium, potassium, calcium, or gold salt. Also within the scope of the invention are salts formed with pharmaceutically acceptable amines such as ammonia, alkyl amines, hydroxyalkylamines, and N-methylglycamine. A compound of Formula (I) can be treated with an acid to form acid addition salts. Examples of such acids include hydrochloric acid, hydrobromic acid, hydroiodic acid, sulfuric acid, methanesulfonic acid, phosphoric acid, p-bromophenyl-sulfonic acid, carbonic acid, succinic acid, citric acid, benzoic acid, oxalic acid, malonic acid, salicylic acid, malic acid, fumaric acid, ascorbic acid, maleic acid, acetic acid, and other mineral and organic acids well known to those skilled in the art. The acid addition salts can be prepared by treating a compound of Formula (I) in its free base form with a sufficient amount of an acid (e.g., hydrochloric acid) to produce an acid addition salt (e.g., a hydrochloride salt). The acid addition salt can be converted back to its free base form by treating the salt with a suitable dilute aqueous basic solution (e.g., sodium hydroxide, sodium bicarbonate, potassium carbonate, or ammonia). Compounds of Formula (I) can also be, e.g., in a forM achiral compounds, racemic mixtures, optically active compounds, pure diastereomers, or a mixture of diastereomers.

Compounds of Formula (I) exhibit surprisingly high affinity to the TGFβ family type I receptors, Alk5 and/or Alk4, e.g., with IC₅₀ and Kᵢ values of less than 10 μM under conditions as described below in Examples 81 and 83, respectively. Some compounds of Formula (I) exhibit IC₅₀ and Kᵢ values of less than 1 μM (such as below 50 nM).

Compounds of Formula (I) can also be modified by appending appropriate functionalities to enhance selective biological properties. Such modifications are known in the art and include those that increase biological penetration into a given biological system (e.g., blood, lymphatic system, central nervous system), increase oral availability, increase solubility to allow administration by injection, alter metabolism, and/or alter rate of excretion. Examples of these modifications include, but are not limited to, esterification with polyethylene glycols, derivatization with pivolates or fatty acid substituents, conversion to carbamates, hydroxylation of aromatic rings, and heteroatom-substitution in aromatic rings.

The present invention also features a pharmaceutical composition comprising a compound of Formula (I) (or a combination of two or more compounds of Formula (I)) and at least one pharmaceutically acceptable carrier. Also included in the present invention is a medicament composition including any of the compounds of Formula (I), alone or in a combination, together with a suitable excipient.

The invention also features a method of inhibiting the TGFβ family type I receptors, Alk5 and/or Alk4 (e.g., with an IC₅₀ value of less than 10 μM; such as, less than 1 μM; and for example, less than 5 nM) in a cell, including the step of contacting the cell with an effective amount of one or more compounds of Formula (I). Also within the scope of the invention is a method of inihibiting the TGFβ and/or activin signaling pathway in a cell or in a subject (e.g., a mammal such as a human), including the step of contacting the cell with or administering to the subject an effective amount of one or more of the compounds of Formula

Also within the scope of the present invention is a method of treating a subject or preventing a subject from suffering a condition characterized by or resulted from an elevated level of TGFβ and/or activin activity. The method includes the step of administering to the subject an effective amount of one or more of a compound of Formula (I). The conditions include an accumulation of excess extracellular matrix; a fibrotic condition (which can be induced by drug or radiation), e.g., scleroderma, lupus nephritis, connective tissue disease, wound healing, surgical scarring, spinal cord injury, CNS scarring, acute lung injury, pulmonary fibrosis (such as idiopathic pulmonary fibrosis and radiation-induced pulmonary fibrosis), chronic obstructive pulmonary disease, adult respiratory distress syndrome, acute lung injury, drug-induced lung injury, glomerulonephritis, diabetic nephropathy, hypertension-induced nephropathy, alimentary track or gastrointestinal fibrosis, renal fibrosis, hepatic or biliary fibrosis, liver cirrhosis, primary biliary cirrhosis, cirrhosis due to fatty liver disease (alcoholic and nonalcoholic steatosis), primary sclerosing cholangitis, restenosis, cardiac fibrosis, opthalmic scarring, fibrosclerosis, fibrotic cancers, fibroids, fibroma, fibroadenomas, fibrosarcomas, transplant arteriopathy, and keloid); TGFβ-induced growth or metastasis of tumor/cancer cells; and carcinomas (e.g., squamous cell carcinomas, multiple myeloma, melanoma, glioma, glioblastomas, leukemia, sarcomas, leiomyomas, mesothelioma, and carcinomas of the lung, breast, ovary, cervix, liver, biliary tract, gastrointestinal tract, pancreas, prostate, and head and neck); and other conditions such as cachexia, hypertension, ankylosing spondylitis, demyelination in multiple sclerosis, cerebral angiopathy and Alzheimer's disease.

For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference. As described herein, compounds of the invention may optionally be substituted with one or more substituents, such as are illustrated generally above, or as exemplified by particular classes, subclasses, and species of the invention.

As used herein the term “aliphatic” encompasses the terms alkyl, alkenyl, alkynyl, each of which being optionally substituted as set forth below.

As used herein, an “alkyl” group refers to a saturated aliphatic hydrocarbon group containing 1-10 (e.g., 1-8, 1-6, or 1-4) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl. An alkyl group can be substituted (i.e., optionally substituted) with one or more substituents such as halo; cycloaliphatic (e.g., cycloalkyl or cycloalkenyl); heterocycloaliphatic (e.g., heterocycloalkyl or heterocycloalkenyl); aryl; heteroaryl; alkoxy; aroyl; heteroaroyl; acyl (e.g., (aliphatic)carbonyl, (cycloaliphatic)carbonyl, or (heterocycloaliphatic)carbonyl); nitro; cyano; amido (e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl); amino (e.g., aliphaticamino, cycloaliphaticamino, or heterocycloaliphaticamino); sulfonyl (e.g., aliphatic-S(O)₂—); sulfinyl; sulfanyl; sulfoxy; urea; thiourea; sulfonamide; sulfamide; oxo; carboxy; carbamoyl; cycloaliphaticoxy; heterocycloaliphaticoxy; aryloxy; heteroaryloxy; aralkyloxy; heteroarylalkoxy; alkoxycarbonyl; alkylcarbonyloxy; or hydroxy. Without limitation, some examples of substituted alkyls include carboxyalkyl (such as HOOC-alkyl, alkoxycarbonylalkyl, and alkylcarbonyloxyalkyl); cyanoalkyl; hydroxyalkyl; alkoxyalkyl; acylalkyl; aralkyl; (alkoxyaryl)alkyl; (sulfonylamino)alkyl (such as alkyl-S(O)₂-aminoalkyl); aminoalkyl; amidoalkyl; (cycloaliphatic)alkyl; or haloalkyl.

As used herein, an “alkenyl” group refers to an aliphatic carbon group that contains 2-10 (e.g., 2-8, 2-6, or 2-4) carbon atoms and at least one double bond. Like an alkyl group, an alkenyl group can be straight or branched. Examples of an alkenyl group include, but are not limited to, allyl, isoprenyl, 2-butenyl, and 2-hexenyl. An alkenyl group can be optionally substituted with one or more substituents such as halo; cycloaliphatic (e.g., cyanoalkyl or cycloalkenyl); heterocycloaliphatic (e.g., heterocycloalkyl or heterocycloalkenyl); aryl; heteroaryl; alkoxy; aroyl; heteroaroyl; acyl (e.g., (aliphatic)carbonyl, (cycloaliphatic)carbonyl, or (heterocycloaliphatic)carbonyl); nitro; cyano; amido (e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl); amino (e.g., aliphaticamino, cycloaliphaticamino, heterocycloaliphaticamino, or aliphaticsulfonylamino); sulfonyl (e.g., alkyl-S(O)₂—, cycloaliphatic-S(O)₂—, or aryl-S(O)₂—); sulfinyl; sulfanyl; sulfoxy; urea; thiourea; sulfonamide; sulfamide; oxo; carboxy; carbamoyl; cycloaliphaticoxy; heterocycloaliphaticoxy; aryloxy; heteroaryloxy; aralkyloxy; heteroaralkoxy; alkoxycarbonyl; alkylcarbonyloxy; or hydroxy. Without limitation, some examples of substituted alkenyls include cyanoalkenyl, alkoxyalkenyl, acylalkenyl, hydroxyalkenyl, aralkenyl, (alkoxyaryl)alkenyl, (sulfonylamino)alkenyl (such as (alkyl-S(O)₂-aminoalkenyl), aminoalkenyl, amidoalkenyl, (cycloaliphatic)alkenyl, or haloalkenyl.

As used herein, an “alkynyl” group refers to an aliphatic carbon group that contains 2-10 (e.g., 2-10, 2-6, or 2-4) carbon atoms and has at least one triple bond. An alkynyl group can be straight or branched. Examples of an alkynyl group include, but are not limited to, propargyl and butynyl. An alkynyl group can be optionally substituted with one or more substituents such as aroyl; heteroaroyl; alkoxy; cycloalkyloxy; heterocycloalkyloxy; aryloxy; heteroaryloxy; aralkyloxy; nitro; carboxy; cyano; halo; hydroxy; sulfo; mercapto; sulfanyl (e.g., aliphatic-S— or cycloaliphatic-S—); sulfinyl (e.g., aliphatic-S(O)— or cycloaliphatic-S(O)—); sulfonyl (e.g., aliphatic-S(O)₂—, aliphaticamino-S(O)₂—, or cycloaliphatic-S(O)₂—); amido (e.g., aminocarbonyl, alkylaminocarbonyl, alkylcarbonylamino, cycloalkylaminocarbonyl, heterocycloalkylaminocarbonyl, cycloalkylcarbonylamino, arylaminocarbonyl, arylcarbonylamino, aralkylcarbonylamino, (heterocycloalkyl)carbonylamino, (cycloalkylalkyl)carbonylamino, heteroaralkylcarbonylamino, heteroarylcarbonylamino or heteroarylaminocarbonyl); urea; thiourea; sulfonamide; sulfamide; alkoxycarbonyl; alkylcarbonyloxy; cycloaliphatic; heterocycloaliphatic; aryl; heteroaryl; acyl (e.g., (cycloaliphatic)carbonyl or (heterocycloaliphatic)carbonyl); amino (e.g., aliphaticamino); sulfoxy; oxo; carbamoyl; (cycloaliphatic)oxy; (heterocycloaliphatic)oxy; or (heteroaryl)alkoxy.

As used herein, an “amido” encompasses both “aminocarbonyl” and “carbonylamino.” These terms when used alone or in connection with another group refers to an amido group such as —N(R^{X})—C(O)—R^{Y} or —C(O)—N(R^{X})₂, when used terminally, and —C(O)—N(R^{X})— or —N(R^{X})—C(O)— when used internally, wherein R^{X} and R^{Y} are defined below. Examples of amido groups include alkylamido (such as alkylcarbonylamino or alkylaminocarbonyl), (heterocycloaliphatic)amido, (heteroaralkyl)amido, (heteroaryl)amido, (heterocycloalkyl)alkylamido, arylamido, aralkylamido, (cycloalkyl)alkylamido, or cycloalkylamido.

As used herein, an “amino” group refers to —NR^{X}R^{Y} wherein each of R^{X} and R^{Y} is independently hydrogen, alkyl, cycloaliphatic, (cycloaliphatic)aliphatic, aryl, araliphatic, heterocycloaliphatic, (heterocycloaliphatic)aliphatic, heteroaryl, carboxy, sulfanyl, sulfinyl, sulfonyl, (aliphatic)carbonyl, (cycloaliphatic)carbonyl, ((cycloaliphatic)aliphatic)carbonyl, arylcarbonyl, (araliphatic)carbonyl, (heterocycloaliphatic)carbonyl, ((heterocycloaliphatic)aliphatic)carbonyl, (heteroaryl)carbonyl, or (heteroaraliphatic)carbonyl, each of which being defined herein and being optionally substituted. Examples of amino groups include alkylamino, dialkylamino, or arylamino. When the term “amino” is not the terminal group (e.g., alkylcarbonylamino), it is represented by —NR^{X}—. R^{X} has the same meaning as defined above.

As used herein, an “aryl” group used alone or as part of a larger moiety as in “aralkyl”, “aralkoxy”, or “aryloxyalkyl” refers to monocyclic (e.g., phenyl); bicyclic (e.g., indenyl, naphthalenyl, tetrahydronaphthyl, tetrahydroindenyl); and tricyclic (e.g., fluorenyl tetrahydrofluorenyl, or tetrahydroanthracenyl, anthracenyl) ring systems in which the monocyclic ring system is aromatic or at least one of the rings in a bicyclic or tricyclic ring system is aromatic. The bicyclic and tricyclic groups include benzofused 2-3 membered carbocyclic rings. For example, a benzofused group includes phenyl fused with two or more C₄₋₈ carbocyclic moieties. An aryl is optionally substituted with one or more substituents including aliphatic (e.g., alkyl, alkenyl, or alkynyl); cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic ring of a benzofused bicyclic or tricyclic aryl); nitro; carboxy; amido; acyl (e.g., aliphaticcarbonyl, (cycloaliphatic)carbonyl, ((cycloaliphatic)aliphatic)carbonyl, (araliphatic)carbonyl, (heterocycloaliphatic)carbonyl, ((heterocycloaliphatic)aliphatic)carbonyl, or (heteroaraliphatic)carbonyl); sulfonyl (e.g., aliphatic-S(O)₂— or amino-S(O)₂—); sulfinyl (e.g., aliphatic-S(O)— or cycloaliphatic-S(O)—); sulfanyl (e.g., aliphatic-S—); cyano; halo; hydroxy; mercapto; sulfoxy; urea; thiourea; sulfonamide; sulfamide; or carbamoyl. Alternatively, an aryl can be unsubstituted.

Non-limiting examples of substituted aryls include haloaryl (e.g., mono-, di (such as p,m-dihaloaryl), and (trihalo)aryl); (carboxy)aryl (e.g., (alkoxycarbonyl)aryl, ((aralkyl)carbonyloxy)aryl, and (alkoxycarbonyl)aryl); (amido)aryl (e.g., (aminocarbonyl)aryl, (((alkylamino)alkyl)aminocarbonyl)aryl, (alkylcarbonyl)aminoaryl, (arylaminocarbonyl)aryl, and (((heteroaryl)amino)carbonyl)aryl); aminoaryl (e.g., ((alkylsulfonyl)amino)aryl or ((dialkyl)amino)aryl); (cyanoalkyl)aryl; (alkoxy)aryl; (sulfonamide)aryl (e.g., (aminosulfonyl)aryl); (alkylsulfonyl)aryl; (cyano)aryl; (hydroxyalkyl)aryl; ((alkoxy)alkyl)aryl; (hydroxy)aryl, ((carboxy)alkyl)aryl; (((dialkyl)amino)alkyl)aryl; (nitroalkyl)aryl; (((alkylsulfonyl)amino)alkyl)aryl; ((heterocycloaliphatic)carbonyl)aryl; ((alkylsulfonyl)alkyl)aryl; (cyanoalkyl)aryl; (hydroxyalkyl)aryl; (alkylcarbonyl)aryl; alkylaryl; (trihaloalkyl)aryl; p-amino-m-alkoxycarbonylaryl; p-amino-m-cyanoaryl; p-halo-m-aminoaryl; or (m-(heterocycloaliphatic)-o-(alkyl))aryl.

As used herein, an “araliphatic” such as an “aralkyl” group refers to an aliphatic group (e.g., a C₁₋₄ alkyl group) that is substituted with an aryl group. “Aliphatic,” “alkyl,” and “aryl” are defined herein. An example of an araliphatic such as an aralkyl group is benzyl.

As used herein, an “aralkyl” group refers to an alkyl group (e.g., a C1-4 alkyl group) that is substituted with an aryl group. Both “alkyl” and “aryl” have been defined above. An example of an aralkyl group is benzyl. An aralkyl is optionally substituted with one or more substituents such as aliphatic (e.g., alkyl, alkenyl, or alkynyl, including carboxyalkyl, hydroxyalkyl, or haloalkyl such as trifluoromethyl); cycloaliphatic (e.g., cycloalkyl or cycloalkenyl); (cycloalkyl)alkyl; heterocycloalkyl; (heterocycloalkyl)alkyl; aryl; heteroaryl; alkoxy; cycloalkyloxy; heterocycloalkyloxy; aryloxy; heteroaryloxy; aralkyloxy; heteroaralkyloxy; aroyl; heteroaroyl; nitro; carboxy; alkoxycarbonyl; alkylcarbonyloxy; amido (e.g., aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, or heteroaralkylcarbonylamino); cyano; halo; hydroxy; acyl; mercapto; alkylsulfanyl; sulfoxy; urea; thiourea; sulfonamide; sulfamide; oxo; or carbamoyl.

As used herein, a “bicyclic ring system” includes 8-12 (e.g., 9, 10, or 11) membered structures that form two rings, wherein the two rings have at least one atom in common (e.g., 2 atoms in common). Bicyclic ring systems include bicycloaliphatics (e.g., bicycloalkyl or bicycloalkenyl), bicycloheteroaliphatics, bicyclic aryls, and bicyclic heteroaryls.

As used herein, a “cycloaliphatic” group encompasses a “cycloalkyl” group and a “cycloalkenyl” group, each of which being optionally substituted as set forth below.

As used herein, a “cycloalkyl” group refers to a saturated carbocyclic mono- or bicyclic (fused or bridged) ring of 3-10 (e.g., 5-10) carbon atoms. Examples of cycloalkyl groups include cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, bicyclo[3.3.2.]decyl, bicyclo[2.2.2]octyl, adamantyl, azacycloalkyl, or ((aminocarbonyl)cycloalkyl)cycloalkyl. A “cycloalkenyl” group, as used herein, refers to a non-aromatic carbocyclic ring of 3-10 (e.g., 4-8) carbon atoms having one or more double bonds. Examples of cycloalkenyl groups include cyclopentenyl, 1,4-cyclohexa-di-enyl, cycloheptenyl, cyclooctenyl, hexahydro-indenyl, octahydro-naphthyl, cyclohexenyl, cyclopentenyl, bicyclo[2.2.2]octenyl, or bicyclo[3.3.1]nonenyl. A cycloalkyl or cycloalkenyl group can be optionally substituted with one or more substituents such as aliphatic (e.g., alkyl, alkenyl, or alkynyl]; cycloaliphatic; (cycloaliphatic)aliphatic; heterocycloaliphatic; (heterocycloaliphatic) aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; amido (e.g., (aliphatic)carbonylamino, (cycloaliphatic)carbonylamino, ((cycloaliphatic)aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, or (heteroaraliphatic)carbonylamino); nitro; carboxy (e.g., HOOC—, alkoxycarbonyl, or alkylcarbonyloxy); acyl (e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, or (heteroaraliphatic)carbonyl); cyano; halo; hydroxy; mercapto; sulfonyl (e.g., alkyl-S(O)₂— and aryl-S(O)₂—); sulfinyl (e.g., alkyl-S(O)—); sulfanyl (e.g., alkyl-S—); sulfoxy; urea; thiourea; sulfonamide; sulfamide; oxo; or carbamoyl.

As used herein, “cyclic moiety” includes cycloaliphatic, heterocycloaliphatic, aryl, or heteroaryl, each of which has been defined previously.

As used herein, the term “heterocycloaliphatic” encompasses a heterocycloalkyl group and a heterocycloalkenyl group, each of which being optionally substituted as set forth below.

As used herein, a “heterocycloalkyl” group refers to a 3-10 membered mono- or bicylic (fused or bridged) (e.g., 5- to 10-membered mono- or bicyclic) saturated ring structure, in which one or more of the ring atoms is a heteroatom (e.g., N, O, S, or combinations thereof). Examples of a heterocycloalkyl group include piperidyl, piperazyl, tetrahydropyranyl, tetrahydrofuryl, 1,4-dioxolanyl, 1,4-dithianyl, 1,3-dioxolanyl, oxazolidyl, isoxazolidyl, morpholinyl, thiomorpholyl, octahydrobenzofuryl, octahydrochromenyl, octahydrothiochromenyl, octahydroindolyl, octahydropyrindinyl, decahydroquinolinyl, octahydrobenzo[b]thiopheneyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.0^{3,7}]nonyl. A monocyclic heterocycloalkyl group can be fused with a phenyl moiety such as tetrahydroisoquinoline. A “heterocycloalkenyl” group, as used herein, refers to a mono- or bicylic (e.g., 5- to 10-membered mono- or bicyclic) non-aromatic ring structure having one or more double bonds, and wherein one or more of the ring atoms is a heteroatom (e.g., N, O, or S). Monocyclic and bicycloheteroaliphatics are numbered according to standard chemical nomenclature.

A heterocycloalkyl or heterocycloalkenyl group can be optionally substituted with one or more substituents such as aliphatic (e.g., alkyl, alkenyl, or alkynyl); cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; amido (e.g., (aliphatic)carbonylamino, (cycloaliphatic)carbonylamino, ((cycloaliphatic) aliphatic)carbonylamino, (aryl)carbonylamino, (araliphatic)carbonylamino, (heterocycloaliphatic)carbonylamino, ((heterocycloaliphatic) aliphatic)carbonylamino, (heteroaryl)carbonylamino, or (heteroaraliphatic)carbonylamino); nitro; carboxy (e.g., HOOC—, alkoxycarbonyl, or alkylcarbonyloxy); acyl (e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, or (heteroaraliphatic)carbonyl); nitro; cyano; halo; hydroxy; mercapto; sulfonyl (e.g., alkylsulfonyl or arylsulfonyl); sulfinyl (e.g., alkylsulfinyl); sulfanyl (e.g., alkylsulfanyl); sulfoxy; urea; thiourea; sulfonamide; sulfamide; oxo; or carbamoyl.

A “heteroaryl” group, as used herein, refers to a monocyclic, bicyclic, or tricyclic ring system having 4 to 15 ring atoms wherein one or more of the ring atoms is a heteroatom (e.g., N, O, S, or combinations thereof) and in which the monocyclic ring system is aromatic or at least one of the rings in the bicyclic or tricyclic ring systems is aromatic. A heteroaryl group includes a benzofused ring system having 2 to 3 rings. For example, a benzofused group includes benzo fused with one or two 4 to 8 membered heterocycloaliphatic moieties (e.g., indolizyl, indolyl, isoindolyl, 3H-indolyl, indolinyl, benzo[b]furyl, benzo[b]thiophenyl, quinolinyl, or isoquinolinyl). Some examples of heteroaryl are azetidinyl, pyridyl, 1H-indazolyl, furyl, pyrrolyl, thienyl, thiazolyl, oxazolyl, imidazolyl, tetrazolyl, benzofuryl, isoquinolinyl, benzthiazolyl, xanthene, thioxanthene, phenothiazine, dihydroindole, benzo[1,3]dioxole, benzo[b]furyl, benzo[b]thiophenyl, indazolyl, benzimidazolyl, benzthiazolyl, puryl, cinnolyl, quinolyl, quinazolyl, cinnolyl, phthalazyl, quinazolyl, quinoxalyl, isoquinolyl, 4H-quinolizyl, benzo-1,2,5-thiadiazolyl, or 1,8-naphthyridyl.

Without limitation, monocyclic heteroaryls include furyl, thiophenyl, 2H-pyrrolyl, pyrrolyl, oxazolyl, thazolyl, imidazolyl, pyrazolyl, isoxazolyl, isothiazolyl, 1,3,4-thiadiazolyl, 2H-pyranyl, 4-H-pranyl, pyridyl, pyridazyl, pyrimidyl, pyrazolyl, pyrazyl, or 1,3,5-triazyl. Monocyclic heteroaryls are numbered according to standard chemical nomenclature.

Without limitation, bicyclic heteroaryls include indolizyl, indolyl, isoindolyl, 3H-indolyl, indolinyl, benzo[b]furyl, benzo[b]thiophenyl, quinolinyl, isoquinolinyl, indolizyl, isoindolyl, indolyl, benzo[b]furyl, bexo[b]thiophenyl, indazolyl, benzimidazyl, benzthiazolyl, purinyl, 4H-quinolizyl, quinolyl, isoquinolyl, cinnolyl, phthalazyl, quinazolyl, quinoxalyl, 1,8-naphthyridyl, or pteridyl. Bicyclic heteroaryls are numbered according to standard chemical nomenclature.

A heteroaryl is optionally substituted with one or more substituents such as aliphatic (e.g., alkyl, alkenyl, or alkynyl); cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic or heterocyclic ring of a bicyclic or tricyclic heteroaryl); carboxy; amido; acyl (e.g., aliphaticcarbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl); sulfonyl (e.g., aliphatic-S(O)₂— or amino-S(O)₂—); sulfinyl (e.g., aliphatic-S(O)—); sulfanyl (e.g., aliphatic-S—); nitro; cyano; halo; hydroxy; mercapto; sulfoxy; urea; thiourea; sulfonamide; sulfamide; or carbamoyl. Alternatively, a heteroaryl can be unsubstituted.

Non-limiting examples of substituted heteroaryls include (halo)heteroaryl (e.g., mono- and di-(halo)heteroaryl); (carboxy)heteroaryl (e.g., (alkoxycarbonyl)heteroaryl); cyanoheteroaryl; aminoheteroaryl (e.g., ((alkylsulfonyl)amino)heteroaryl and((dialkyl)amino)heteroaryl); (amido)heteroaryl (e.g., aminocarbonylheteroaryl, ((alkylcarbonyl)amino)heteroaryl, ((((alkyl)amino)alkyl)aminocarbonyl)heteroaryl, (((heteroaryl)amino)carbonyl)heteroaryl, ((heterocycloaliphatic)carbonyl)heteroaryl, and ((alkylcarbonyl)amino)heteroaryl); (cyanoalkyl)heteroaryl; (alkoxy)heteroaryl; (sulfonamide)heteroaryl (e.g., (aminosulfonyl)heteroaryl); (sulfonyl)heteroaryl (e.g., (alkylsulfonyl)heteroaryl); (hydroxyalkyl)heteroaryl; (alkoxyalkyl)heteroaryl; (hydroxy)heteroaryl; ((carboxy)alkyl)heteroaryl; [((dialkyl)amino)alkyl)heteroaryl; (heterocycloaliphatic)heteroaryl; (cycloaliphatic)heteroaryl; (nitroalkyl)heteroaryl; (((alkylsulfonyl)amino)alkyl)heteroaryl; ((alkylsulfonyl)alkyl)heteroaryl; (cyanoalkyl)heteroaryl; (acyl)heteroaryl (e.g., (alkylcarbonyl)heteroaryl); (alkyl)heteroaryl, and (haloalkyl)heteroaryl (e.g., trihaloalkylheteroaryl).

A “heteroaraliphatic (such as a heteroaralkyl group) as used herein, refers to an aliphatic group (e.g., a C₁₋₄ alkyl group) that is substituted with a heteroaryl group. “Aliphatic,” “alkyl,” and “heteroaryl” have been defined above.

A “heteroaralkyl” group, as used herein, refers to an alkyl group (e.g., a C₁₋₄ alkyl group) that is substituted with a heteroaryl group. Both “alkyl” and “heteroaryl” have been defined above. A heteroaralkyl is optionally substituted with one or more substituents such as alkyl (e.g., carboxyalkyl, hydroxyalkyl, and haloalkyl such as trifluoromethyl); alkenyl; alkynyl; cycloalkyl; (cycloalkyl)alkyl; heterocycloalkyl; (heterocycloalkyl)alkyl; aryl; heteroaryl; alkoxy; cycloalkyloxy; heterocycloalkyloxy; aryloxy; heteroaryloxy; aralkyloxy; heteroaralkyloxy; aroyl; heteroaroyl; nitro; carboxy; alkoxycarbonyl; alkylcarbonyloxy; aminocarbonyl; alkylcarbonylamino; cycloalkylcarbonylamino; (cycloalkylalkyl)carbonylamino; arylcarbonylamino; aralkylcarbonylamino; (heterocycloalkyl)carbonylamino; (heterocycloalkylalkyl)carbonylamino; heteroarylcarbonylamino; heteroaralkylcarbonylamino; cyano; halo; hydroxy; acyl; mercapto; alkylsulfanyl; sulfoxy; urea; thiourea; sulfonamide; sulfamide; oxo; or carbamoyl.

As used herein, an “acyl” group refers to a formyl group or R^{X}—C(O)— (such as -alkyl-C(O)—, also referred to as “alkylcarbonyl”) where R^{X} and “alkyl” have been defined previously. Acetyl and pivaloyl are examples of acyl groups.

As used herein, an “aroyl” or “heteroaroyl” refers to an aryl-C(O)— or a heteroaryl-C(O)—. The aryl and heteroaryl portion of the aroyl or heteroaroyl is optionally substituted as previously defined.

As used herein, an “alkoxy” group refers to an alkyl-O— group where “alkyl” has been defined previously.

As used herein, a “carbamoyl” group refers to a group having the structure —O—CO—NR^{X}R^{Y} or —NR^{X}—CO—O—R^{Z} wherein R^{X} and R^{Y} have been defined above and R^{Z} can be aliphatic, aryl, araliphatic, heterocycloaliphatic, heteroaryl, or heteroaraliphatic.

As used herein, a “carboxy” group refers to —COOH, —COOR^{X}, —OC(O)H, —OC(O)R^{X} when used as a terminal group; or —OC(O)— or —C(O)O— when used as an internal group.

As used herein, a “haloaliphatic” group refers to an aliphatic group substituted with 1-3 halogen. For instance, the term haloalkyl includes the group —CF₃.

As used herein, a “mercapto” group refers to —SH.

As used herein, a “sulfo” group refers to —SO₃H or —SO₃R^{X} when used terminally or —S(O)₃— when used internally.

As used herein, a “sulfamide” group refers to the structure —NR^{X}—S(O)₂—NR^{Y}R^{Z} when used terminally and —NR^{X}—S(O)₂—NR^{Y}— when used internally, wherein R^{X}, R^{Y}, and R^{Z} have been defined above.

As used herein, a “sulfonamide” group refers to the structure —S(O)₂—NR^{X}R^{Y} or —NR^{X}—S(O)₂—R^{Z} when used terminally; or —S(O)₂—NR^{X}— or —NR^{X}—S(O)₂— when used internally, wherein R^{X}, R^{Y}, and R^{Z} are defined above.

As used herein a “sulfanyl” group refers to —S—R^{X} when used terminally and —S— when used internally, wherein R^{X} has been defined above. Examples of sulfanyls include aliphatic-S—, cycloaliphatic-S—, aryl-S—, or the like.

As used herein a “sulfinyl” group refers to —S(O)—R^{X} when used terminally and —S(O)—when used internally, wherein R^{X} has been defined above. Exemplary sulfinyl groups include aliphatic-S(O)—, aryl-S(O)—, (cycloaliphatic(aliphatic))-S(O)—, cycloalkyl-S(O)—, heterocycloaliphatic-S(O)—, heteroaryl-S(O)—, or the like.

As used herein, a “sulfonyl” group refers to —S(O)₂—R^{X} when used terminally and —S(O)₂-when used internally, wherein R^{X} has been defined above. Exemplary sulfonyl groups include aliphatic-S(O)₂—, aryl-S(O)₂—, (cycloaliphatic(aliphatic))-S(O)₂—, cycloaliphatic-S(O)₂—, heterocycloaliphatic-S(O)₂—, heteroaryl-S(O)₂—, (cycloaliphatic(amido(aliphatic)))-S(O)₂-or the like.

As used herein, a “sulfoxy” group refers to —O—SO—R^{X} or —SO—O—R^{X}, when used terminally and —O—S(O)— or —S(O)—O— when used internally, where R^{X} has been defined above.

As used herein, a “halogen” or “halo” group refers to fluorine, chlorine, bromine or iodine.

As used herein, an “alkoxycarbonyl,” which is encompassed by the term carboxy, used alone or in connection with another group refers to a group such as alkyl-O—C(O)—.

As used herein, an “alkoxyalkyl” refers to an alkyl group such as alkyl-O-alkyl-, wherein alkyl has been defined above.

As used herein, a “carbonyl” refer to —C(O)—.

As used herein, an “oxo” refers to ═O.

As used herein, an “aminoalkyl” refers to the structure (R^{X})₂N-alkyl-.

As used herein, a “cyanoalkyl” refers to the structure (NC)-alkyl-.

As used herein, a “urea” group refers to the structure —NR^{X}—CO—NR^{Y}R^{Z} and a “thiourea” group refers to the structure —NR^{X}—CS—NR^{Y}R^{Z} when used terminally and —NR^{X}—CO—NR^{Y}— or —NR^{X}—CS—NR^{Y}— when used internally, wherein R^{X}, R^{Y}, and R^{Z} have been defined above.

As used herein, a “guanidino” group refers to the structure —N═C(N(R^{X}R^{Y}))N(R^{X}R^{Y}) or —N(R^{X})C═(N(R^{X}))N(R^{X}R^{Y}) wherein R^{X} and R^{Y} have been defined above.

As used herein, the term “amidino” group refers to the structure —C═(NR^{X})N(R^{X}R^{Y}) wherein R^{X} and R^{Y} have been defined above.

In general, the term “vicinal” refers to the placement of substituents on a group that includes two or more carbon atoms, wherein the substituents are attached to adjacent carbon atoms.

In general, the term “geminal” refers to the placement of substituents on a group that includes two or more carbon atoms, wherein the substituents are attached to the same carbon atom.

The terms “terminally” and “internally” refer to the location of a group within a substituent. A group is terminal when the group is present at the end of the substituent not further bonded to the rest of the chemical structure. Carboxyalkyl, i.e., R^{X}O(O)C-alkyl is an example of a carboxy group used terminally. A group is internal when the group is present in the middle of a substituent to at the end of the substituent bound to the rest of the chemical structure. Alkylcarboxy (e.g., alkyl-C(O)O— or alkyl-OC(O)—) and alkylcarboxyaryl (e.g., alkyl-C(O)O-aryl- or alkyl-O(CO)-aryl-) are examples of carboxy groups used internally.

As used herein, “cyclic group” includes mono-, bi-, and tri-cyclic ring systems including cycloaliphatic, heterocycloaliphatic, aryl, or heteroaryl, each of which has been previously defined.

As used herein, a “bridged bicyclic ring system” refers to a bicyclic heterocyclicalipahtic ring system or bicyclic cycloaliphatic ring system in which the rings have at least two common atoms. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.2.3]nonyl, 2-oxabicyclo[2.2.2]octyl, 1-azabicyclo[2.2.2]octyl, 3-azabicyclo[3.2.1]octyl, and 2,6-dioxatricyclo[3.3.1.03,7]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfonamide, sulfamide, oxo, or carbamoyl.

As used herein, an “aliphatic chain” refers to a branched or straight aliphatic group (e.g., alkyl groups, alkenyl groups, or alkynyl groups). A straight aliphatic chain has the structure —(CH₂)ᵥ—, where v is 1-6. A branched aliphatic chain is a straight aliphatic chain that is substituted with one or more aliphatic groups. A branched aliphatic chain has the structure —(CHQ)ᵥ— where Q is hydrogen or an aliphatic group; however, Q shall be an aliphatic group in at least one instance. The term aliphatic chain includes alkyl chains, alkenyl chains, and alkynyl chains, where alkyl, alkenyl, and alkynyl are defined above.

The phrase “optionally substituted” is used interchangeably with the phrase “substituted or unsubstituted.” As described herein, compounds of the invention can optionally be substituted with one or more substituents, such as are illustrated generally above, or as exemplified by particular classes, subclasses, and species of the invention. As described herein, the variables R₁, R₂, R₃, R₄, and other variables contained in Formula (I) encompass specific groups, such as alkyl and aryl. Unless otherwise noted, each of the specific groups for the variables R₁, R₂, R₃, R₄, and other variables contained therein can be optionally substituted with one or more substituents described herein. Each substituent of a specific group is further optionally substituted with one to three of halo, cyano, hydroxy, amino, nitro, aryl, haloalkyl, and alkyl. For instance, an alkyl group can be substituted with alkylsulfanyl and the alkylsulfanyl can be optionally substituted with one to three of halo, cyano, hydroxy, amino, nitro, aryl, haloalkyl, and alkyl. As an additional example, the cycloalkyl portion of a (cycloalkyl)carbonylamino can be optionally substituted with one to three of halo, cyano, alkoxy, hydroxy, nitro, haloalkyl, and alkyl. When two alkoxy groups are bound to the same atom or adjacent atoms, the two alkxoy groups can form a ring together with the atom(s) to which they are bound.

In general, the term “substituted,” whether preceded by the term “optionally” or not, refers to the replacement of hydrogen radicals in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, e.g., both rings share one common atom. As one of ordinary skill in the art will recognize, combinations of substituents envisioned by this invention are those combinations that result in the formation of stable or chemically feasible compounds.

The phrase “stable or chemically feasible,” as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

As used herein, an effective amount is defined as the amount required to confer a therapeutic effect on the treated patient, and is typically determined based on age, surface area, weight, and condition of the patient. The interrelationship of dosages for animals and humans (based on milligrams per meter squared of body surface) is described by Freireich et al., Cancer Chemother. Rep., 50: 219 (1966). Body surface area may be approximately determined from height and weight of the patient. See, e.g., Scientific Tables, Geigy Pharmaceuticals, Ardsley, N.Y., 537 (1970). As used herein, “patient” refers to a mammal, including a human.

An antagonist, as used herein, is a molecule that binds to the receptor without activating the receptor. It competes with the endogenous ligand(s) or substrate(s) for binding site(s) on the receptor and, thus inhibits the ability of the receptor to transduce an intracellular signal in response to endogenous ligand binding.

As compounds of Formula (I) are antagonists of TGFβ receptor type I (Alk5) and/or activin receptor type I (Alk4), these compounds are useful in inhibiting the consequences of TGFβ and/or activin signal transduction such as the production of extracellular matrix (e.g., collagen and fibronectin), the differentiation of stromal cells to myofibroblasts, and the stimulation of and migration of inflammatory cells. Thus, compounds of Formula (I) inhibit pathological inflammatory and fibrotic responses and possess the therapeutic utility of treating and/or preventing disorders or diseases for which reduction of TGFβ and/or activin activity is desirable (e.g., various types of fibrosis or progressive cancers). In addition, the compounds of Formula (I) are useful for studying and researching the role of TGFβ receptor type I (Alk5) and/or activin receptor type I (Alk4), such as their role in cellular processes, for example, signal transduction, production of extracellular matrix, the differentiation of stromal cells to myofibroblasts, and the stimulation of and migration of inflammatory cells.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a 13C- or 14C-enriched carbon are within the scope of this invention. Such compounds are useful, for example, as analytical tools or probes in biological assays.

### DETAILED DESCRIPTION OF THE INVENTION

In general, the invention features compounds of Formula (I), which exhibit surprisingly high affinity for the TGFβ family type I receptors, Alk 5 and/or Alk 4.

### Synthesis of the Compounds of Formula (I)

Compound of the invention may be prepared by known methods and as further illustrated below.

In one method, compounds of this intention may be prepared as illustrated in Scheme 1.

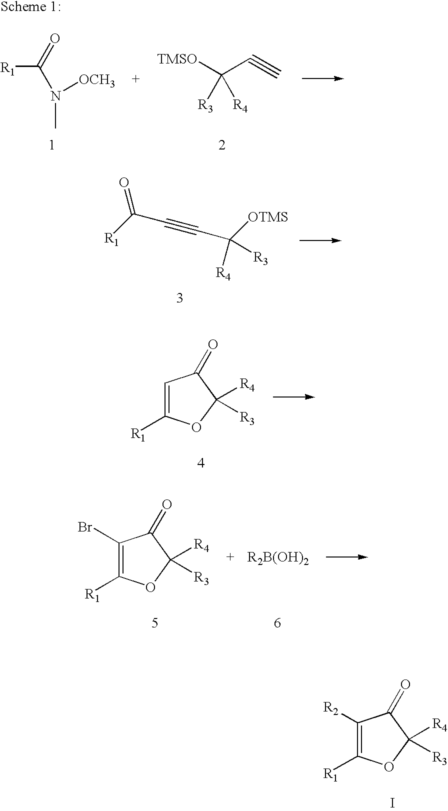

Referring to Scheme 1, a methoxymethyl amide 1 reacts with a trimethylsilyl protected propynol 2 in the presence of a strong base such as, e.g., lithium diisopropylamide (LDA) to provide a yneone 3. Removal of the trimethylsilyl group and cyclization with a secondary amine, e.g. diethylamine, provides a furanone 4. Bromination of the furanone 4 with, e.g., N-bromsuccinimide provides an intermediate bromofuranone 5 which is subsequently coupled with an aryl boronic acid 6 in the presence of a catalyst such as, e.g., palladium acetate, to provide a compound of the invention I.

An alternative method for preparing the compounds of this invention is illustrated in Scheme 2.

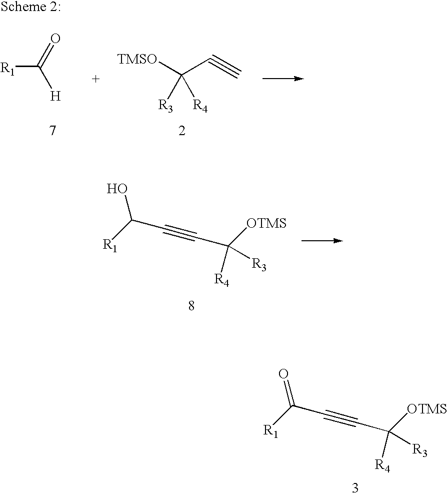

Referring to Scheme 2, an aryl aldehyde 7 reacts with a trimethylsilyl protected propynol 2 in the presence of a strong base such as, e.g., lithium diisopropylamide (LDA) to provide a protected intermediate 8. Oxidation of the intermediate 8 with, e.g., Dess-Martin periodinane provides a yneone 3 which is then converted to compound I as described in Scheme 1.

An alternative method for the addition of the R₂ moiety is illustrated in Scheme 3.

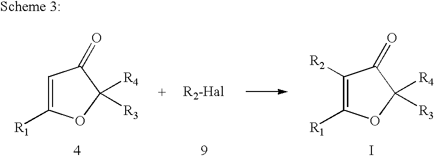

In Scheme 3, a furanone 4 reacts with an aryl halide 9, wherein Hal is iodo or bromo, to provide compound of Formula I.

In some embodiments, wherein R₃ and R₄ together with the atom to which they are attached form a heterocycloaliphatic ring, for example, a piperidine ring, further modification of the ring nitrogen can be achieved, e.g., as illustrated in Scheme 4.

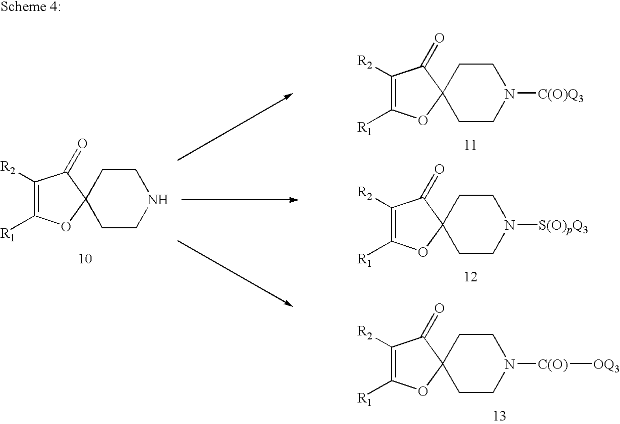

Referring to Scheme 4, reaction of a piperidine compound 10 with acyl or aryl acid halide Q₃C(O)-Hal provides compounds of formula 11. Likewise, reaction of the piperidine compound 10 with a sulfenyl, sulfinyl or sulfonyl halide Q₃S(O)ₚ— gives compounds of formula 12. Reaction of the piperidine compound 10 with an alkoxy carbonyl halide provides carbamate compounds of formula 13.

The spiro-piperidine 10 of Scheme 4 may be prepared, e.g., as illustrated below in Scheme 5

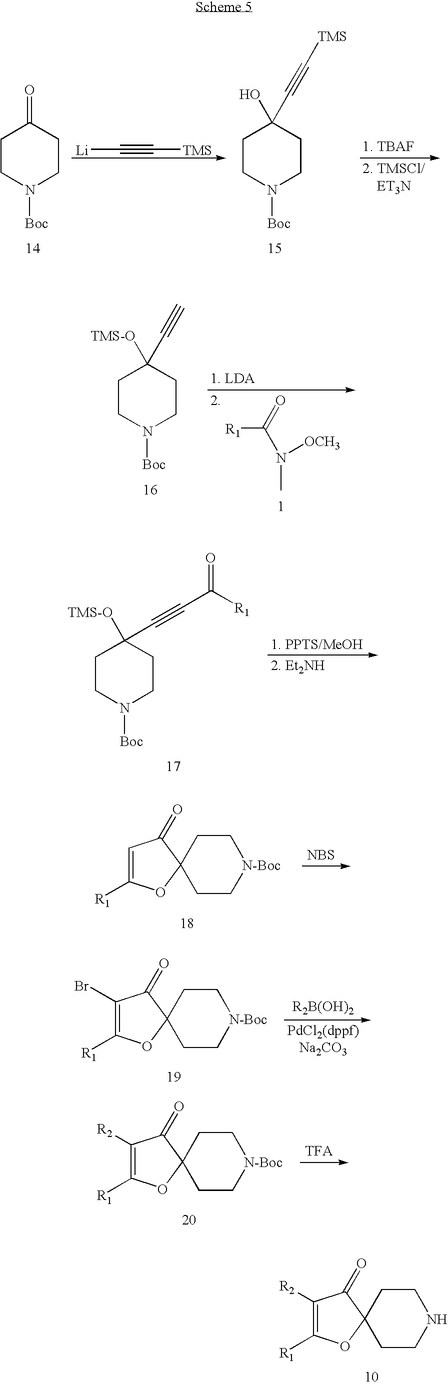

Referring to Scheme 5, the protected piperidone 14 is reacted with ((trimethylsilyl)ethynyl)lithium to provide the adduct 15. Deprotection of the acetylenic trimethylsilyl compound 15 with, for example, tetrabutylammonium fluoride followed by protection of the hydroxygroup provides the acetylenic-piperidine 16. Reaction of compound 16 with a methoxymethyl amide 1 in the presence of a strong base such as, for example, LDA provides the adduct 17. Removal of the trimethylsilyl protecting group followed by cyclization as previously described provides a furanone 18. Bromination of the furanone 18 provides a bromofuranone 19 which undergoes a Suzuki reaction to give the furanone 20. Removal of the Boc protecting group provides the piperidine-furanone 10.

In some embodiments, wherein R₃ and R₄ together with the atom to which they are attached form, for example, a 6-membered ring containing a hydroxy group, further modifications may be made, e.g., as illustrated below in Scheme 6.

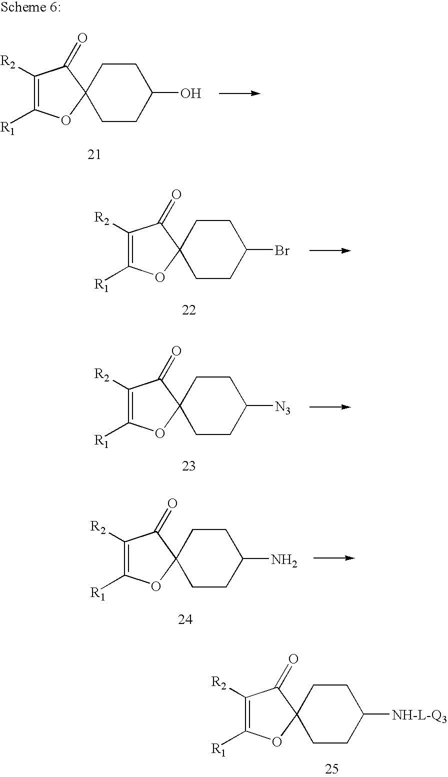

Referring to Scheme 6, the hydroxy group of compound 21 may be substituted with bromo to give a bromo compound 22 by reaction with carbontetrabromide and triphenylphosphine. Displacement of bromo in compound 22 with, for example, sodium azide provides an azido compound 23 which may be subsequently reduced using know conditions such as reaction with triphenyl phosphine to the amino compound 24. Further modification of compound 24 as described above for the piperidine compound 10 provides compounds of the invention of formula 25.

In some embodiments, wherein R₃ and R₄ together with the atom to which they are attached form, for example, a 6-membered ring containing a keto group, further modifications may be made, e.g., as illustrated in Scheme 7.

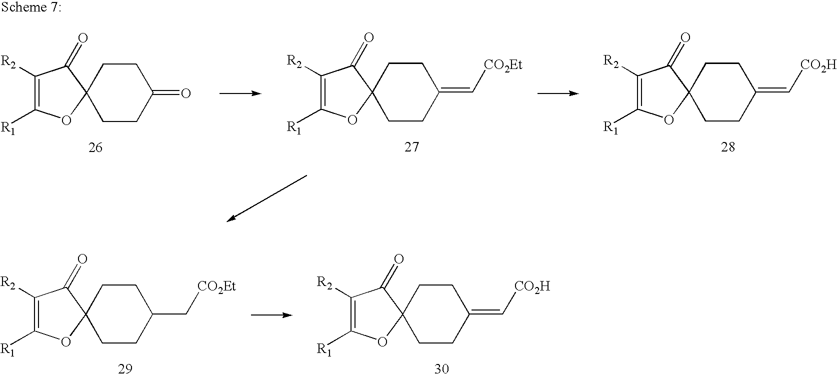

Referring to Scheme 7, a ketone 26 may be reacted with a carbethoxy ylid to provide an ester 27. Hydrolysis of the ester of 27 provides an acid 28 which may be converted to acid derivatives such as, for example, amides, ureas, esters and carbamates. Alternatively, reduction of the ester 27 with, for example, hydrogen in the presence of a palladium catalyst provides a saturated ester 29 which may be hydrolysed to an acid 30. The acid 30 may be further modified to provide additional derivatives as described above.

In some embodiments, wherein R₃ and R₄ together with the atom to which they are attached form, for example, a 6-membered ring containing an aldehyde group, further modifications may be made, e.g., as illustrated in Scheme 8.

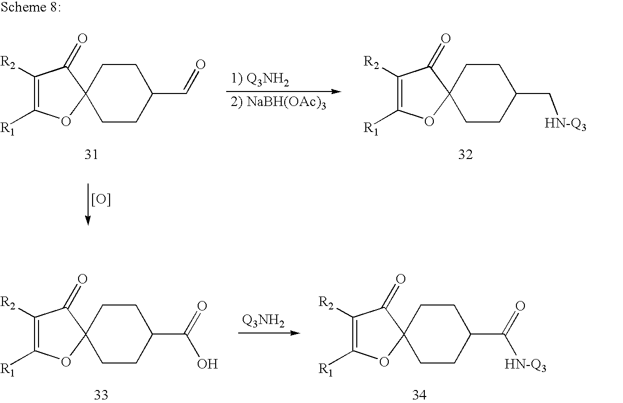

Referring to Scheme 8, an aldehyde 31 may be reductively aminated with an amine Q₃NH₂ and a reducing agent such as, for example, sodium triacetoxyborohydride to provide an amine of formula 32. Alternatively, the aldehyde 31 may be oxidized with, for example, chromic acid in acetone (Jones' reagent) to provide an acid 33. Further derivitization provides, for example, amides of formula 34.

The aldehyde 31 shown in Scheme 7 may be prepared as illustrated in Scheme 9.

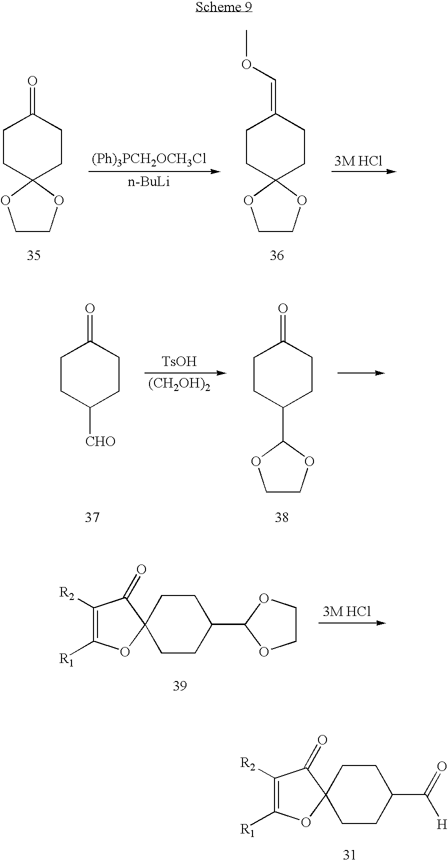

Referring to Scheme 9, the ketal 35 is reacted with (methoxymethyl)triphenyl-phosphonium chloride in the presence of butyl lithium to provide the methoxymethylene compound 36. Hydrolysis with 3M hydrochloric acid provides the aldehyde-ketone 37 which may be selectively protected with ethylene glycol in the presence of toluenesulfonic acid to provide the acetal-ketone 38. The furanone 39 may subsequently be prepared from compound 38 using methods as described by Schemes 1, 2, and 3. Hydrolysis of the acetal 39 provides the aldehyde 31.

Methods for the preparation of starting materials illustrated in Schemes 4, 5, 6, 7 and 8 are provided in the examples below.

### Uses of Compounds of Formula (I)

As discussed above, hyperactivity of the TGFβ family signaling pathways can result in excess deposition of extracellular matrix and increased inflammatory responses, which can then lead to fibrosis in tissues and organs (e.g., lung, kidney, and liver) and ultimately result in organ failure. See, e.g., Border, W. A. and Ruoslahti E. J. Ctn. Invest. 90:1-7 (1992) and Border, W. A. and Noble, N. A. N Engl. J. Med. 331: 1286-1292 (1994). Studies have been shown that the expression of TGFβ and/or activin mRNA and the level of TGFβ and/or activin are increased in patients suffering from various fibrotic disorders, e.g., fibrotic kidney diseases, alcohol-induced and autoimmune hepatic fibrosis, myelofibrosis, bleomycin-induced pulmonary fibrosis, and idiopathic pulmonary fibrosis. Elevated TGFβ and/or activin is has also been demonstrated in cachexia, demyelination of neurons in multiple sclerosis, Alzheimer's disease, cerebral angiopathy and hypertension.

Compounds of Formula (I), which are antagonists of the TGFβ family type I receptors Alk5 and/or Alk4, and inhibit TGFβ and/or activin signaling pathway, are therefore useful for treating and/or preventing fibrotic disorders or diseases mediated by an increased level of TGFβ and/or activin activity. As used herein, a compound inhibits the TGFβ family signaling pathway when it binds (e.g., with an IC₅₀ value of less than 10 μM; such as, less than 1 μM; and for example, less than 5 nM) to a receptor of the pathway (e.g., Alk 5 and/or Alk 4), thereby competing with the endogenous ligand(s) or substrate(s) for binding site(s) on the receptor and reducing the ability of the receptor to transduce an intracellular signal in response to the endogenous ligand or substrate binding. The aforementioned disorders or diseases include any condition (a) marked by the presence of an abnormally high level of TGFβ and/or activin; and/or (b) an excess accumulation of extracellular matrix; and/or (c) an increased number and synthetic activity of myofibroblasts. These disorders or diseases include, but are not limited to, fibrotic conditions such as scleroderma, glomerulonephritis, diabetic nephropathy, lupus nephritis, hypertension-induced nephropathy, ocular or corneal scarring, alimentary track or gastrointestinal fibrosis, renal fibrosis, hepatic or biliary fibrosis, acute lung injury, pulmonary fibrosis (such as idiopathic pulmonary fibrosis and radiation-induced pulmonary fibrosis), post-infarction cardiac fibrosis, fibrosclerosis, fibrotic cancers, fibroids, fibroma, fibroadenomas, and fibrosarcomas. Other fibrotic conditions for which preventive treatment with compounds of Formula (I) can have therapeutic utility include radiation-induced fibrosis, chemotherapy-induced fibrosis, and surgically-induced scarring including surgical adhesions, laminectomy, and coronary restenosis.

Increased TGFβ activity is also found to manifest in patients with progressive cancers. Studies have shown that in many cancers, the tumor cells, stromal cells, and/or other cells within a tumor generally overexpress TGFβ. This leads to stimulation of angiogenesis and cell motility, suppression of the immune system, and/or increased interaction of tumor cells with the extracellular matrix. See, e.g., Hojo, M. et al., Nature 397: 530-534 (1999) and Lammerts E. et al., Int. J. Cancer 102: 453-462 (2002). As a result, the tumors grow more readily, become more invasive and metastasize to distant organs. See, e.g., Maehara, Y. et al., J. Clin. Oncol. 17: 607-614 (1999) and Picon, A. et al., Cancer Epidemiol. Biomarkers Prey. 7: 497-504 (1998). Thus, compounds of Formula (I), which are antagonists of the TGFβ type I receptor and inhibit TGFβ signaling pathways, are also useful for treating and/or preventing various cancers which overexpress TGFβ or benefit from TGFβ's above-mentioned pro-tumor activities. Such cancers include carcinomas of the lung, breast, liver, biliary tract, gastrointestinal tract, head and neck, pancreas, prostate, cervix as well as multiple myeloma, melanoma, glioma, and glioblastomas.

Importantly, it should be pointed out that because of the chronic, and in some cases localized, nature of disorders or diseases mediated by overexpression of TGFβ and/or activin (e.g., fibrosis or cancers), small molecule treatments (such as treatment disclosed in the present invention) are favored for long-term treatment.

Not only are compounds of Formula (I) useful in treating disorders or diseases mediated by high levels of TGFβ and/or activin activity, these compounds can also be used to prevent the same disorders or diseases. It is known that polymorphisms leading to increased TGFβ and/or activin production have been associated with fibrosis and hypertension. Indeed, high serum TGFβ levels are correlated with the development of fibrosis in patients with breast cancer who have received radiation therapy, chronic graft-versus-host-disease, idiopathic interstitial pneumonitis, veno-occlusive disease in transplant recipients, and peritoneal fibrosis in patients undergoing continuous ambulatory peritoneal dialysis. Thus, the levels of TGFβ and/or activin in serum and of TGFβ and/or activin mRNA in tissue can be measured and used as diagnostic or prognostic markers for disorders or diseases mediated by overexpression of TGFβ and/or activin, and polymorphisms in the gene for TGFβ that determine the production of TGFβ and/or activin can also be used in predicting susceptibility to disorders or diseases. See, e.g., Blobe, G. C. et al., N Engl. J. Med., 342(18): 1350-1358 (2000); Matsuse, T. et al., Am. J. Respir. Cell Mol. Biol., 13: 17-24 (1995); Inoue, S. et al., Biochem. Biophys. Res. Comm., 205: 441-448 (1994); Matsuse, T. et al, Am. J. Pathol., 148: 707-713 (1996); De Bleser et al., Hepatology, 26: 905-912 (1997); Pawlowski, J. E., et al., J. Clin. Invest., 100: 639-648 (1997); and Sugiyama, M. et al., Gastroenterology, 114: 550-558 (1998).

### Administration of Compounds of Formula (I)

As defined above, an effective amount is the amount required to confer a therapeutic effect on the treated patient. For a compound of Formula (I), an effective amount can range, for example, from about 1 mg/kg to about 150 mg/kg (e.g., from about 1 mg/kg to about 100 mg/kg). Effective doses will also vary, as recognized by those skilled in the art, dependant on route of administration, excipient usage, and the possibility of co-usage with other therapeutic treatments including use of other therapeutic agents and/or radiation therapy.

Compounds of Formula (I) can be administered in any manner suitable for the administration of pharmaceutical compounds, including, but not limited to, pills, tablets, capsules, aerosols, suppositories, liquid formulations for ingestion or injection or for use as eye or ear drops, dietary supplements, and topical preparations. The pharmaceutically acceptable compositions include aqueous solutions of the active agent, in an isotonic saline, 5% glucose or other well-known pharmaceutically acceptable excipient. Solubilizing agents such as cyclodextrins, or other solubilizing agents well-known to those familiar with the art, can be utilized as pharmaceutical excipients for delivery of the therapeutic compounds. As to route of administration, the compositions can be administered orally, intranasally, transdermally, intradermally, vaginally, intraaurally, intraocularly, buccally, rectally, transmucosally, or via inhalation, implantation (e.g., surgically), or intravenous administration. The compositions can be administered to an animal (e.g., a mammal such as a human, non-human primate, horse, dog, cow, pig, sheep, goat, cat, mouse, rat, guinea pig, rabbit, hamster, gerbil, or ferret, or a bird, or a reptile, such as a lizard).

Optionally, compounds of Formula (I) can be administered in conjunction with one or more other agents that inhibit the TGFβ signaling pathway or treat the corresponding pathological disorders (e.g., fibrosis or progressive cancers) by way of a different mechanisM action. Examples of these agents include angiotensin converting enzyme inhibitors, nonsteroid and steroid anti-inflammatory agents, immunotherapeutics, chemotherapeutics, as well as agents that antagonize ligand binding or activation of the TGFβ receptors, e.g., anti-TGFβ, anti-TGFβ receptor antibodies, or antagonists of the TGFβ type II receptors. Compounds of Formula (I) can also be administered in conjunction with other treatments, e.g., radiation.

### Preparations and Examples

The invention will be further described in the following examples, which do not limit the scope of the invention described in the claims. Nomenclature is consistent with ChemDraw Ultra, version 9.0.1, Cambridgesoft.com.

### Example 1

### 2-([1,2,4]triazolo[1,5-a]pyridin-6-yl)-8-hydroxy-3-m-tolyl-1-oxaspiro[4.5]dec-2-en-4-one}

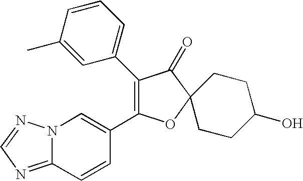

### Step 1: [1,2,4]Triazolo[1,5-a]pyridine-6-carboxylic acid ethyl ester

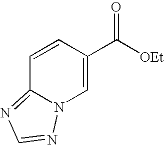

6-Iodo-[1,2,4]triazolo[1,5-a]pyridine (20.0 g, 0.0816 mol), sodium acetate (33.5 g, 0.408 mol; Aldrich), [1,1′-Bis(diphenylphosphino)ferrocene]dichloropalladium(II), complex with dichloromethane (1:1) (2.0 g, 0.0024 mol; Aldrich), and ethanol (600 mL, 10 mol; Fisher) were added to a Parr reactor and stirred. A carbon monoxide (CO) tank was connected to the Parr reactor and 55 psi of CO was added to the reactor after one vent to clear the reactor. The reactor was then heated to 90° C. and stirred overnight. The reactor was cooled and LC-MS showed no starting material and clean conversion to the product (2.08 min, ES+/192.19). The reaction contents were removed from the bomb and concentrated. The residue was dissolved in ethyl acetate and the solution washed with saturated sodium bicarbonate and brine, dried over MgSO₄ and concentrated to dryness. The residue was purified on a CombiFlash silica gel column with 0-100% ethyl acetate in hexane to give the desired product as a tan solid (8.6 g, 55%).

¹H-NMR (CDCl₃, 300 MHz) δ d 9.25 (1H, s), 8.38 (1H, s), 8.06 (1H, d), 7.74 (1H, d), 4.39 (2H, qt), 1.37 (3H, t)

### Step 2: N-methoxy-N-methyl-[1,2,4]triazolo[1,5-a]pyridine-6-carboxamide

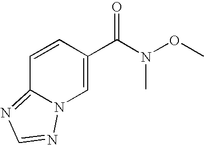

A mixture of [1,2,4]triazolo[1,5-a]pyridine-6-carboxylic acid methyl ester (9 g, 0.05 mol) in 2 M sodium hydroxide (100 mL) and tetrahydrofuran (200 mL, Acros) was stirred overnight when TLC showed no more starting ester. The mixture was slowly acidified with 3M HCl until a white milky ppt formed and persisted, diluted with water and the ppt was collected and dried and used without further purification.

To a suspension of the above product in methylene chloride (250 mL, 3.9 mol; Fisher) and N,N-dimethylformamide (0.04 mL, 0.0005 mol; Acros) was slowly added oxalyl chloride (11 mL, 0.13 mol; Aldrich) and the mixture stirred for 2 h. The solvents were evaporated and a suspension of N-methoxymethanamine hydrochloride (13 g; Acros) in pyridine (200 mL, Aldrich) was added and the mixture stirred overnight. TLC and LC-MS showed the formation of the desired product (1.17 min, ES+/207.06). The mixture was diluted with methylene chloride and water, the phases separated and the organic phase dried over MgSO₄ and concentrated. The residue was purified on a CombiFlash silica gel column with 0-100% ethyl acetate to give the desired product as a white crystalline solid (8.66 g, 80%).

¹H-NMR: (CDCl₃, 300 MHz) δ 9.13 (1H, s), 8.35 (1H, s), 7.99 (1H, d), 7.72 (1H, d), 3.56 (3H, s), 3.36 (3H, s).

### Step 3: 4-(tert-Butyl-dimethyl-silanyloxy)-cyclohexanone

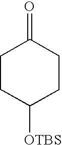

To a solution of 1,4-cyclohexanediol (20.0 g, 0.172 mol; Aldrich) and 1H-Imidazole (33 g, 0.48 mol; Aldrich) in N,N-dimethylformamide (250 mL, 3.2 mol; Acros) was added a solution of tert-butyldimethylsilyl chloride (20.0 g, 0.133 mol; Aldrich) in N,N-dimethylformamide (100 mL, 1 mol; Acros) via a dropping funnel at 0° C. The mixture was was allowed to warm to room temperature and stirred overnight. TLC (ethyl acetate/hexane 1:4) showed almost complete reaction and the mixture was partitioned between water and ether, the organic phase dried over MgSO₄ and concentrated in vacuo.

The crude product from above was dissolved in methylene chloride (500 mL, 8 mol; Acros) and mixed with Celite (100 g). Pyridinium chlorochromate (41 g, 0.19 mol; Aldrich) was added in 5 portions at 0° C. The cooling bath was removed and the mixture stirred for 3 hours. The mixture was filtered thru a short silica gel column and the column washed with methylene chloride. The filtrates were concentrated and further purified on CombiFlash silica gel column with 0-50% ethyl acetate in hexane to give the desired product as a colorless oil.

### Step 4: 4-(tert-Butyl-dimethyl-silanyloxy)-1-ethynyl-cyclohexanol

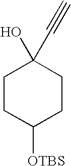

To a solution of N,N-diisopropylamine (12 mL, 0.087 mol; Aldrich) in anhydrous tetrahydrofuran (100 mL, 1 mol; Acros) was added dropwise 1.6 M n-Butyllithium in hexane (54 mL; Aldrich) at −78° C. and the mixture stirred for 30 minutes. (Trimethylsilyl)acetylene (12 mL, 0.087 mol; Aldrich) was then added and stirred for 1 h before a solution of 4-(tert-Butyl-dimethyl-silanyloxy)-cyclohexanone (16.5 g, 0.0722 mol) in THF (20 mL) was added and stirred for an hour at −78° C. and then warmed to room temperature. TLC showed complete reaction. The mixture was partitioned between saturated ammonium chloride and ether, the organic phase was dried over MgSO₄ and concentration in vacuo to give a colorless oil.

The above crude product was dissolved in methanol (200 mL, 5 mol; Fisher), potassium carbonate (10 g, 0.07 mol; Fisher) was added and the mixture stirred for 30 minutes. TLC showed complete reaction. The mixture was concentrated and the residue partioned between methylene chloride and water, the organic phase dried over MgSO₄ and concentrated in vacuo and the residue purified on CombiFlash silica gel column with 0-50% ethyl acetate to give the desired product as a colorless oil.

¹H NMR (CDCl₃, 300 MHz): δ 3.80 (br, 1H), 2.63 (s, 1H), 1.94-1.85 (m, 2H), 1.74-1.65 (m, 6H), 0.84 (s, 9H), 0.00 (s, 6H)

### Step 5: 4-(tert-Butyl-dimethyl-silanyloxy)-1-ethynyl-1-trimethylsilanyloxycyclohexane

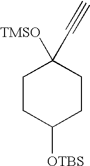

To a solution of [A]-4-(tert-butyldimethylsilanyloxy)-1-ethynylcyclohexanol (13.1 g, 0.0515 mol) in methylene chloride (400 mL, 6 mol; Fisher) was added 4-dimethylaminopyridine (60 mg, 0.0005 mol; Aldrich), triethylamine (22 mL, 0.15 mol; Aldrich) and chlorotrimethylsilane (9.8 mL, 0.077 mol; Aldrich). The mixture was stirred for 30 min when TLC showed complete reaction. The mixture was partioned between methylene chloride and water, the organic phase dried over MgSO₄ and concentrated in vacuo and the residue purified on a short silica gel column with 100% ether to give the desired product as a colorless oil.

¹H NMR (CDCl₃, 300 MHz): δ 3.50 (br, 1H), 2.31 (s, 1H), 1.81-1.73 (m, 2H), 1.47-1.36 (m, 6H), 0.70 (s, 9H), 0.00 (s, 9H), −0.15 (s, 6H)

### Step 6: 3-([1,2,4]triazolo[1,5-a]pyridin-6-yl)-1-(4-(tert-butyldimethylsilyloxy)-1-(trimethylsilyloxy)cyclohexyl)prop-2-yn-1-one

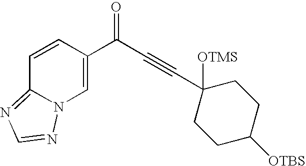

To a solution of N,N-diisopropylamine (5.6 mL, 0.040 mol; Aldrich) in tetrahydrofuran (100 mL, 1 mol; Acros) was added dropwise 1.6 M n-butyllithium in hexane (23 mL; Aldrich) at −78° C. and the mixture stirred for 30 min. A solution of 4-(tert-butyldimethylsilyloxy)-1-ethynyl-1-trimethylsilanyloxycyclohexane (10.0 g, 0.0306 mol) in tetrahydrofuran (50 mL, 0.6 mol; Acros) was then added slowly to at −78° C. and the mixture stirred for 30 min. A solution of [1,2,4]triazolo[1,5-a]pyridine-6-carboxylic acid, methoxy-methyl-amide (8.6 g, 0.042 mol) in tetrahydrofuran (100 mL, 1 mol; Acros) was then added slowly and the mixture stirred for 30 minutes. The cooling bath was removed and the mixture allowed to warm to room temperature for 1 hour. TLC showed complete reaction. The mixture was portioned between ether and water, the organic phase dried over MgSO₄ and concentrated in vacuo and the residue and purified on a short silica gel column with 100% methylene chloride, then 100% ethyl acetate to give the desired product as an orange foamy solid.

¹H NMR (CDCl₃, 300 MHz): [symbol] 9.33 (d, 1H), 8.45 (d, 1H), 7.77 (d, 1H), 3.78 (br, 1H), 2.20 (m, 2H), 1.8-1.6 (m, 6H), 0.83 (s, 9H), 0.18 (s, 9H), 0.00 (s, 6H)

### Step 7: 2-([1,2,4]triazolo[1,5-a]pyridin-6-yl)-8-(tert-butyldimethylsilyloxy)-1-oxaspiro[4.5]dec-2-en-4-one

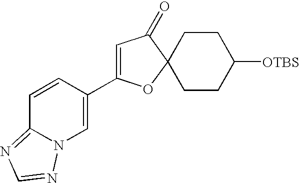

A solution of 3-[4-(tert-butyldimethylsilanyloxy)-1-trimethylsilanyloxy-cyclohexyl]-1-[1,2,4]triazolo[1,5-a]pyridin-6-yl-propynone (12.5 g, 0.0265 mol) and potassium carbonate (0.92 g, 0.0066 mol; Fisher) in methanol (300 mL, 7 mol; Fisher) was stirred overnight when TLC showed complete conversion. The mixture was concentrated and the residue purified on CombiFlash silica gel column with 0-100% ethyl acetate in hexane to give the desired product C as a yellow foamy solid. (9.8 g, 92%, ES+/400.42)

### Step 8: 2-([1,2,4]triazolo[1,5-a]pyridin-6-yl)-8-hydroxy-3-m-tolyl-1-oxaspiro[4.5]dec-2-en-4-one

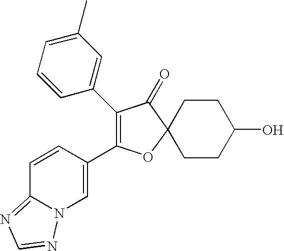

Cesium acetate (1920 mg, 0.0100 mol) was dried under vacuum (120 microns) at 125° C. for 2 hours. Then palladium acetate (11.2 mg, 0.0000500 mol), tris(4-trifluoromethylphenyl)phosphine (93.4 mg, 0.000200 mol), and anhydrous N,N-dimethylformamide (4 mL, 0.05 mol) were added and the mixture stirred for 30 min. In a separate flask under an atmosphere of nitrogen, 2-([1,2,4]triazolo[1,5-a]pyridin-6-yl)-8-(tert-butyldimethylsilyloxy)-1-oxaspiro[4.5]dec-2-en-4-one (2000.0 mg, 0.0050055 mol) and 3-iodotoluene (0.768 mL, 0.00601 mol) was mixed in anhydrous N,N-dimethylformamide (10 mL) then transferred into the catalyst mixture under nitrogen. The mixture was heated at 125° C. overnight. LC-MS showed the formation of the desired product (4.68 min, ES+/490.38) and some of desilyated product (2.72 min, ES+/376.35). The mixture was cooled to room temperature, partitioned between ethyl acetate and water, the organic phase dried over MgSO₄ and concentrated. The crude product was treated with 2N HCl in THF to remove the TBS, the mixture was neutralized with 1N NaOH and extracted with ethyl acetate. The organic phase was dried over MgSO₄ and purified on CombiFlash silica gel column with 0-100% ethyl acetate in hexane, then 0-15% MeOH in ethyl acetate to give the desired product (0.3 g, 20%).

¹H NMR (CDCl₃, 300 MHz): [symbol] 9.07 (s, 1H), 8.40 (s, 1H), 7.72 (d, 1H), 7.63 (d, 1H), 7.25-6.9 (m, 4H), 3.78 (br, 1H), 2.26 (s, 3H), 2.1-1.5 (m, 8H)

MS ESI: 376.35 (M+1).

### Example 2

### 8-Bromo-3-m-tolyl-2-[1,2,4]triazolo[1,5-a]pyridin-6-yl-1-oxa-spiro[4.5]dec-2-en-4-one

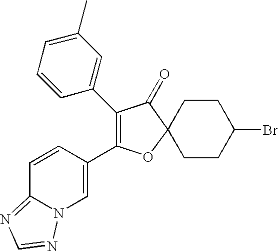

To a solution of 8-hydroxy-3-m-tolyl-2-[1,2,4]triazolo[1,5-a]pyridin-6-yl-1-oxa-spiro[4.5]dec-2-en-4-one (1.1 g, 0.0029 mol) and carbon tetrabromide (1.1 g, 0.0032 mol; Aldrich) in anhydrous tetrahydrofuran (60 mL, 0.7 mol; Acros) was added triphenylphosphine (0.84 g, 0.0032 mol; Aldrich) and the mixture stirred overnight. LC-MS showed the formation of the desired product (3.63 min, ES+/438.07&440.39). The mixture was partitioned between water and ethyl acetate, the organic phase dried over MgSO₄ and purified on CombiFlash silica gel column with 0-100% ethyl acetate in methylene chloride to give the desired product (950 mg, 74%).

¹H NMR (CDCl₃, 300 MHz): [symbol] 8.99 (s, 1H), 8.33 (s, 1H), 7.60 (d, 1H), 7.55 (d, 1H), 7.26-6.99 (m, 4H), 4.69 (br, 1H), 2.26 (s, 3H), 2.4-2.0 (m, 8H)

MS ESI: 438.07/440.39 (M+1).

### Example 3

### 2-([1,2,4]triazolo[1,5-a]pyridin-6-yl)-8-azido-3-m-tolyl-1-oxaspiro[4.5]dec-2-en-4-one

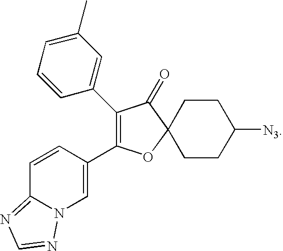

A mixture of 8-bromo-3-m-tolyl-2-[1,2,4]triazolo[1,5-a]pyridin-6-yl-1-oxa-spiro[4.5]dec-2-en-4-one (0.9 g, 0.002 mol) and sodium azide (1.33 g, 0.0205 mol; Aldrich) in N,N-dimethylformamide (10 mL, 0.1 mol; Acros) was heated at 60° C. overnight. LC-MS showed formation of the desired product (3.55 min, ES+/401.26). The mixture was cooled to room temperature and water was added and the resultant precipitate collected, washed with water and dried. (750 mg, 90%).

¹H NMR (CDCl₃, 300 MHz): [symbol] 9.14 (s, 1H), 8.65 (s, 1H), 7.95 (d, 1H), 7.82 (d, 1H), 7.36-7.04 (m, 4H), 3.62 (m, 1H), 2.38 (s, 3H), 2.25-2.10 (m, 2H), 2.00-1.81 (m, 6H)

MS ESI: 401.26 (M+1).

### Example 4

### 2-([1,2,4]triazolo[1,5-a]pyridin-6-yl)-8-amino-3-m-tolyl-1-oxaspiro[4.5]dec-2-en-4-one.

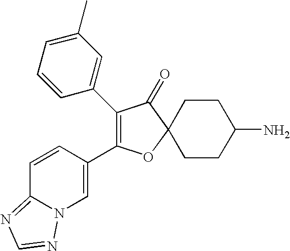

A solution of 8-azido-3-m-tolyl-2-[1,2,4]triazolo[1,5-a]pyridin-6-yl-1-oxa-spiro[4.5]dec-2-en-4-one (750.0 mg, 0.001873 mol) and triphenylphosphine (580 mg, 0.0022 mol; Aldrich) in tetrahydrofuran (20 mL, 0.2 mol; Acros) was stirred overnight when LC-MS showed complete conversion of the azide to the corresponding phosphine imide (3.47 min, ES+/635.38). 2 N HCl (20 mL) was added and the mixture refluxed overnight. LC-MS showed the formation of the desired product (2.31 min, ES+/375.30) and remaining phosphine imide. Reflux was continued for 24 hours when LC-MS showed reaction is almost complete. The mixture was cooled to room temperature and diluted with 1N HCl and extracted with ethyl acetate. The aqueous layer was neutralized with sodium carbonate and extracted with ethyl acetate. The organic phase was dried over MgSO₄ and purified on a short silica gel column with ethyl acetate/MeOH/sat. NH₄OH (85:10:5) to give the desired product as a yellow solid (580 mg, 83%). ¹H NMR (CDCl₃, 300 MHz): [symbol] 9.01 (s, 1H), 8.34 (s, 1H), 7.59 (s, 4H), 7.25-6.90 (m, 4H), 2.85 (m, 1H), 2.28 (s, 3H), 2.00-1.41 (m, 8H)

MS ESI: 375.30 (M+1).

### Example 5

### N-(2-([1,2,4]triazolo[1,5-a]pyridin-6-yl)-4-oxo-3-m-tolyl-1-oxaspiro[4.5]dec-2-en-8-yl)methanesulfonamide

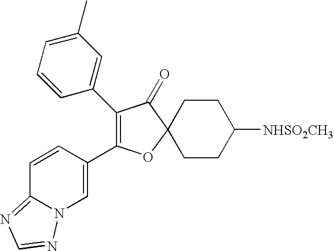

To solution of 8-amino-3-m-tolyl-2-[1,2,4]triazolo[1,5-a]pyridin-6-yl-1-oxa-spiro[4.5]dec-2-en-4-one (80 mg, 0.0002 mol) and N,N-diisopropylethylamine (200 uL, 0.001 mol; Aldrich) in tetrahydrofuran (5 mL, 0.06 mol; Acros) was added methanesulfonyl chloride (25 uL, 0.00032 mol; Aldrich) and the mixture stirred overnight. LC-MS showed complete reaction (2.88 min, ES+/453.17). The mixture was partitioned between ethyl acetate and water, the organic phase dried over MgSO₄ and purified on Gilson HPLC to give the desired product (25 mg, 20%).

¹H NMR (CDCl₃, 300 MHz): [symbol] 9.19 (s, 1H), 8.54 (s, 1H), 7.85 (d, 1H), 7.76 (d, 1H), 7.35-7.06 (m, 4H), 3.56 (m, 1H), 3.03 (s, 3H), 2.28 (s, 3H), 2.27-1.80 (m, 8H)

MS ESI: 453.17 (M+1).

### Example 6

### 11-m-Tolyl-10-[1,2,4]triazolo[1,5-a]pyridin-6-yl-1,4,9-trioxa-dispiro[4.2.4.2]tetradec-10-en-12-one.

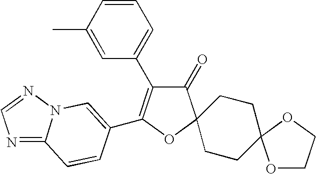

### Step 1: 8-Trimethylsilanylethynyl-1,4-dioxa-spiro[4.5]decan-8-ol

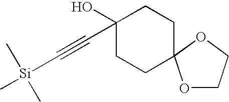

To a solution of (trimethylsilyl)acetylene (14.0 mL, 0.0991 mol; Aldrich) in anhydrous tetrahydrofuran (200.0 mL, 2.466 mol; Acros) was added dropwise a solution of 1.6 M n-Butyllithium in hexane (52.0 mL; Aldrich) at −78° C. and the mixture stirred for 1 h. A solution of 1,4-dioxa-spiro[4.5]decan-8-one (10.0 g, 0.0640 mol; Aldrich) in ahydrous THF (50 mL) was added dropwise at −78° C. and the mixture stirred for 1 h and then allowed to warm to room temperature. The mixture was quenched with sat. NH₄Cl, extracted with diethyl ether and the ether phase dried over MgSO₄. After concentration in vacuo, the residue was purified on a short silica column with 0-20% ethyl acetate in methylene chloride to give the desired product as a white waxy solid.

¹H NMR (CDCl₃, 300 MHz): δ 3.90 (s, 4H), 1.94-1.82 (m, 4H), 1.72 (t, 4H), 0.12 (s, 9H)

### Step 2: 8-Ethynyl-1,4-dioxa-spiro[4.5]decan-8-ol

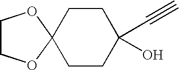

A suspension of 8-trimethylsilanylethynyl-1,4-dioxa-spiro[4.5]decan-8-ol (15.7 g, 0.0617 mol) and potassium carbonate (26.0 g, 0.188 mol; Fisher) in methanol (200.0 mL, 4.937 mol; Fischer) was stirred at room temperature for 1 h until TLC showed complete reaction. The mixture was filtered and the filtrate concentrated. The residue was purified on a short silica gel column with 20-50% ethyl acetate in methylene chloride to give the desired product as a colorless syrup.

¹HNMR (CDCl₃, 300 MHz): 3.77 (s, 4H), 2.34 (s, 1H), 1.87-1.75 (m, 4H), 1.62 (t, 4H)

### Step 3: (8-Ethynyl-1,4-dioxa-spiro[4.5]dec-8-yloxy)-trimethyl-silane

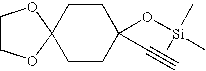

To a solution of 8-ethynyl-1,4-dioxa-spiro[4.5]decan-8-ol (11.1 g, 0.0609 mol) and triethylamine (25 mL, 0.18 mol; Aldrich) and 4-dimethylaminopyridine (0.1 g, 0.001 mol) in methylene chloride (250.0 mL, 3.900 mol; Fisher) was slowly added chlorotrimethylsilane (12 mL, 0.091 mol; Aldrich). The mixture was stirred at room temperature for 5 h when TLC showed complete reaction. The mixture was partitioned between methylene chloride and water, the organic phase dried over MgSO₄ then purified on a short silica gel column with 100% methylene chloride to give the desired product as yellow liquid.

¹HNMR (CDCl₃, 300 MHz): 3.77 (s, 4H), 2.29 (s, 1H), 1.78-1.67 (m, 4H), 1.63-1.51 (m, 4H), 0.00 (s, 9H).

### Step 4: 1-[1,2,4]-Triazolo[1,5-a]pyridin-6-yl-3-(8-trimethylsilanyloxy-1,4-dioxa-spiro[4.5]dec-8-yl)-prop-2-yn-1-ol

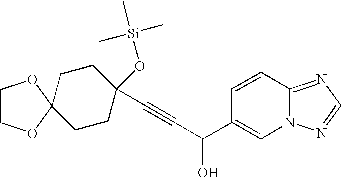

To a solution of N,N-diisopropylamine (4.6 mL, 0.033 mol; Aldrich) in anhydrous THF (100 mL, Acros) was added dropwise 1.6 M n-butyllithium in hexane (19.0 mL; Aldrich) at −78° C. and the mixture stirred for 1 h. To the above solution was added dropwise a solution of (8-ethynyl-1,4-dioxa-spiro[4.5]dec-8-yloxy)-trimethylsilane (6.5 g, 0.026 mol) in anhydrous THF (50 mL, Acros) at −78° C. and the mixture stirred for 1 h. To the above solution was added dropwise a solution of [1,2,4]triazolo[1,5-a]pyridine-6-carbaldehyde (4.1 g, 0.028 mol) in anhydrous THF (150 mL, Acros) at −78° C. and the mixture stirred for 1 h. The mixture was allowed to warm to room temperature then quenched with water and extracted with ethyl acetate (3×). The combined organic phases were washed with saturated ammonium chloride dried over MgSO₄, concentrated and the residue purified on CombiFlash silica gel column with 0-100% ethyl acetate in methylene chloride to give the desired product (2.4 g, 22%) and the desilyated product (4.2 g, 50%). ES+/330.42

### Step 5: 1-([1,2,4]triazolo[1,5-a]pyridin-6-yl)-3-(8-(trimethylsilyloxy)-1,4-dioxaspiro[4.5]decan-8-yl)prop-2-yn-1-one

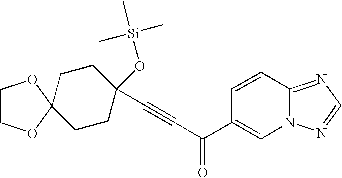

To a solution of 8-(3-Hydroxy-3-[1,2,4]triazolo[1,5-a]pyridin-6-yl-prop-1-ynyl)-1,4-dioxa-spiro[4.5]decan-8-ol (0.44 g, 0.0013 mol) in methylene chloride (100.0 mL, 1.560 mol; Fisher) was added Dess-Martin periodinane (0.85 g, 0.0020 mol; Lancaster) and stirred at room temperature for 15 min. LC-MS showed complete reaction. Worked up with methylene chloride and saturated sodium carbonate and dried over MgSO₄ to give a foamy white solid.

¹H-NMR (CDCl₃, 300 MHz): d 9.33 (s, 1H), 8.42 (s, 1H), 8.09 (d, 1H), 7.90 (d, 1H), 3.88 (s, 4H), 2.19-2.03 (m, 4H), 1.86-1.83 (m, 2H), 1.77-1.75 (m, 2H); ES+/328.15

### Step 6: 10-[1,2,4]Triazolo[1,5-a]pyridin-6-yl-1,4,9-trioxa-dispiro[4.2.4.2]tetradec-10-en-12-one

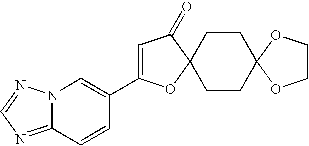

A solution of 1-([1,2,4]triazolo[1,5-a]pyridin-6-yl)-3-(8-(trimethylsilyloxy)-1,4-dioxaspiro[4.5]decan-8-yl)prop-2-yn-1-one (1.3 g, 0.0040 mol) and diethylamine (1.0 mL, 0.0097 mol; Aldrich) in methanol (100 mL, 3 mol; Fisher) was stirred at room temperature for 2 h. LC-MS showed formation of the desired furanone (2.28 min, ES+/328.21). The mixture was concentrated and the residue purified on CombiFlash silica gel column with 0-5% methanol in ethyl acetate to give the desired product as a white solid (0.44 g, 32%).

### Step 7: 11-m-Tolyl-10-[1,2,4]triazolo[1,5-a]pyridin-6-yl-1,4,9-trioxa-dispiro[4.2.4.2]tetradec-10-en-12-one

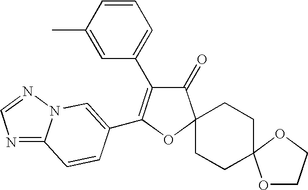

Cesium acetate (79.9 mg, 0.000416 mol) was dried under vacumn (120 microns) at 125° C. for 2 hours in an 8 ml vial fitted with a septum. Palladium acetate (1.37 mg, 6.11E-6 mol; Strem), tris(4-trifluoromethylphenyl)phosphine (11.4 mg, 0.0000244 mol; Strem), and anhydrous N,N-dimethylformamide (0.5 mL, 0.006 mol; Acros) were added and the mixture stirred for 30 min. In a seperate flask under an atmosphere of nitrogen, 10-[1,2,4]Triazolo[1,5-a]pyridin-6-yl-1,4,9-trioxa-dispiro[4.2.4.2]tetradec-10-en-12-one (200 mg, 0.0006 mol) and 3-iodotoluene (0.160 g, 0.000733 mol; Aldrich) was mixed in anhydrous N,N-dimethylformamide (1.3 mL, 0.016 mol; Acros). This mixture was transferred into the catalyst mixture under nitrogen and the mixture heated at 125° C. overnight. LC-MS showed 80% of conversion to the desired product (3.21 min, ES+/418.14). The mixture was cooled, partitioned between ethyl aacetate and water, the organic phase dried over MgSO₄ and purified on CombiFlash silica gel column with 0-100% ethyl acetate in hexane to give the desired product as a white solid (145 mg, 60%).

¹H-NMR (CDCl₃, 300 MHz): δ 8.98 (s, 1H), 8.33 (s, 1H), 7.60 (s, 2H), 7.25-6.98 (m, 4H), 3.94 (s, 4H), 2.27 (s, 3H), 2.19-2.03 (m, 2H), 2.01-1.79 (m, 6H); ES+/418.14

### Example 7

### 2-([1,2,4]triazolo[1,5-a]pyridin-6-yl)-3-m-tolyl-1-oxaspiro[4.5]dec-2-ene-4,8-dione

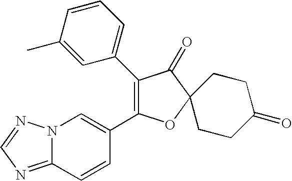

A mixture of 11-m-tolyl-10-[1,2,4]triazolo[1,5-a]pyridin-6-yl-1,4,9-trioxa-dispiro[4.2.4.2]tetradec-10-en-12-one (140 mg, 0.00034 mol), 3 M hydrogen chloride in water (10 mL) and tetrahydrofuran (10 mL, 0.1 mol; Acros) was stirred overnight when LC-MS showed complete reaction. The mixture was concentrated and the residue neutralized with saturated Na₂CO₃ then extracted with ethyl acetate. The organic phase was dried over MgSO₄ and purified on CombiFlash with 0-100% ethyl acetate in hexane to give an 80% pure product which was then further purified on HPLC to give the product as a TFA salt.

¹H-NMR (CDCl₃, 300 MHz): d 9.05 (s, 1H), 8.69 (s, 1H), 7.77 (d, 1H), 7.66 (d, 1H), 7.28-7.01 (m, 4H), 2.36 (s, 3H), 2.78-2.15 (m, 8H) ES+/374.19

### Example 8

### 11-(6-Methyl-pyridin-2-yl)-10-[1,2,4]triazolo[1,5-a]pyridin-6-yl-1,4,9-trioxa-dispiro[4.2.4.2]tetradec-10-en-12-one

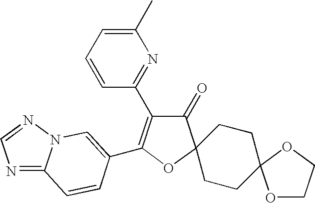

Cesium Acetate (79.9 mg, 0.000416 mol) was dried under vacumn (120 microns) at 125° C. for 2 hours in a 8 ml vial fitted with a septum. Added Palladium Acetate (1.37 mg, 6.11E-6 mol; Strem) and tris(4-trifluoromethylphenyl)phosphine (11.4 mg, 0.0000244 mol; Strem), and anhydrous N,N-dimethylformamide (0.5 mL, 0.006 mol; Acros) were added to a small vial and let stir for 30 min. In a seperate flask under an atmosphere of nitrogen, 10-[1,2,4]-triazolo[1,5-a]pyridin-6-yl-1,4,9-trioxa-dispiro[4.2.4.2]tetradec-10-en-12-one (200 mg, 0.0006 mol) and 2-bromo-6-methylpyridine (0.13 g, 0.00073 mol; Aldrich) were mixed in anhydrous N,N-dimethylformamide (1.3 mL, 0.016 mol; Acros). The mixture was transferred into the catalyst mixture under nitrogen and heated at 125° C. overnight. The mixture was cooled to the room temperature and purified on CombiFlash silica gel column with 0-100% ethyl acetate to give recovered starting material (100 mg, 50%) and the desired product (60 mg, 20%).

ES+/: 419.20

### Example 9

### 2-([1,2,4]triazolo[1,5-a]pyridin-6-yl)-3-(6-methylpyridin-2-yl)-1-oxaspiro[4.5]dec-2-ene-4,8-dione

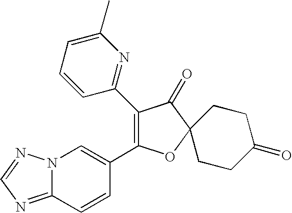

A solution of 11-(6-Methyl-pyridin-2-yl)-10-[1,2,4]triazolo[1,5-a]pyridin-6-yl-1,4,9-trioxa-dispiro[4.2.4.2]tetradec-10-en-12-one (50 mg, 0.0001 mol) in 3 M hydrogen chloride in water (10 mL) and tetrahydrofuran (10 mL, 0.1 mol; Acros) was stirred overnight when LC-MS showed complete reaction (1.23 min, ES+/375.12). The mixture was concentrated and the residue purified on Gilson semi-prep HPLC to give the product as a TFA salt (2 mg, 4%).

¹H-NMR (MeOD, 300 MHz): d 9.57 (s, 1H), 8.69 (s, 1H), 8.42 (d, 1H), 8.35 (d, 1H), 7.98-7.72 (m, 4H), 2.87 (s, 3H), 2.51-2.49 (m, 2H), 2.22-1.98 (m, 6H) ES+/375.12

### Example 10

### Ethyl 2-(2-([1,2,4]triazolo[1,5-a]pyridin-6-yl)-4-oxo-3-m-tolyl-1-oxaspiro[4.5]dec-2-en-8-ylidene)acetate

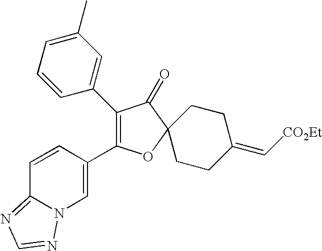

To a stirred solution of ethyl 2-(diethoxyphosphoryl)acetate (269 mg, 1.2 mmol) in THF (5 mL), NaH (27 mg, 1.1 mmol) was added under protection of nitrogen at room temperature (15° C.) and stirred for 30 min at this temperature. A solution of 3-m-tolyl-2-[1,2,4]triazolo[1,5-a]pyridin-6-yl-1-oxa-spiro[4.5]dec-2-ene-4,8-dione (373 mg, 1.0 mmol) in THF (5 mL) was added and stirred for another 30 min at room temperature. The mixture was quenched with NH₄Cl aqueous solution (20 mL) and extracted with methylene chloride (20 mL×2). The combined organic phases were dried over MgSO₄, concentrated and the residue purified on silica gel column using ethylacetate/petroleum ether (¼) as eluant to give ethyl 2-(2-([1,2,4]triazolo[1,5-a]pyridin-6-yl)-4-oxo-3-m-tolyl-1-oxaspiro[4.5]dec-2-en-8-ylidene)acetate (376 mg, Y: 85%) as a yellow solid.

¹H NMR, (400 MHz, CDCl₃) δ 9.14 (s, 1H), 8.55 (s, 1H), 7.92 (d, J=9.2 Hz, 1H), 7.81 (d, J=9.2 Hz, 1H), 7.33-7.04 (m, 4H), 5.82 (s, 1H), 4.21 (q, J=7.0 Hz, 2H), 3.95 (d, J=14.0 Hz, 1H), 2.72-2.51 (m, 3H), 2.36 (s, 3H), 2.15-2.04 (m, 4H), 1.31 (t, J=7.0 Hz, 3H).

MS ESI: 444 (M+1).

### Example 11

### Ethyl 2-(2-([1,2,4]triazolo[1,5-a]pyridin-6-yl)-4-oxo-3-m-tolyl-1-oxaspiro[4.5]dec-2-en-8-yl)acetate

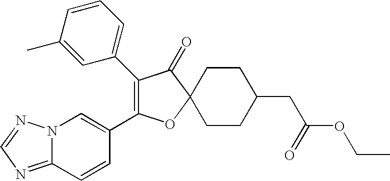

To a stirred solution of ethyl 2-(2-([1,2,4]triazolo[1,5-a]pyridin-6-yl)-4-oxo-3-m-tolyl-1-oxaspiro[4.5]dec-2-en-8-ylidene)acetate (420 mg, 0.95 mmol) in ethyl acetate (15 mL), Pd/C (10%, 100 mg) was added and the mixture hydrogenated at 50° C. and 1 atm H₂ for 10 h. After filtration and concentration, the residue was purified on silica gel column using ethyl acetate/petroleum ether (¼) as eluant to give a mixture (50 mg) of cis and trans isomers as a yellow solid. The isomers were separated by preparative-HPLC to give 18a (12 mg, 3%) and 18b (12 mg, 3%) as yellow solids.

18a: ¹H-NMR, (400 MHz, CDCl₃) δ 9.11 (s, 1H), 8.50 (s, 1H), 7.82 (d, J=8.8 Hz, 1H), 7.72 (d, J=8.8 Hz, 1H), 7.32-7.04 (m, 4H), 4.17 (q, J=7.2 Hz, 2H), 2.74-2.27 (m, 2H), 2.38 (s, 3H), 2.03-1.79 (m, 7H), 1.59-1.49 (m, 2H), 1.28 (t, J=7.0 Hz, 3H). MS ESI: 446 (M+1).

18b: ¹H-NMR, (400 MHz, CDCl₃) δ 9.06 (s, 1H), 8.41 (s, 1H), 7.63 (s, 2H), 7.31-7.05 (m, 4H), 4.17 (q, J=7.2 Hz, 2H), 2.71-2.41 (m, 3H), 2.38 (s, 3H), 2.20-1.84 (m, 9H), 1.29 (t, J=7.0 Hz, 3H). MS ESI: 446 (M+1).

### Example 12

### 2-(2-([1,2,4]triazolo[1,5-a]pyridin-6-yl)-4-oxo-3-m-tolyl-1-oxaspiro[4.5]dec-2-en-8-yl)acetic acid

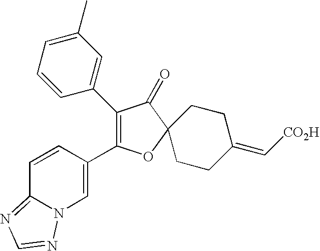

Lithium hydroxide (40 mg, 0.002 mol) was added to a solution of (4-oxo-3-m-tolyl-2-[1,2,4]triazolo[1,5-a]pyridin-6-yl-1-oxa-spiro[4.5]dec-2-en-8-ylidene)-acetic acid ethyl ester (0.34 g, 0.00078 mol) in tetrahydrofuran (10 mL, 0.1 mol). The mixture was stirred for 1 h then partitioned between ethyl acetate and water. The organic phase was purified by HPLC to give the product as a TFA salt (250 mg, 77%).

¹H NMR, (400 MHz, CDCl₃) δ 9.14 (s, 1H), 8.55 (s, 1H), 7.92 (d, J=9.2 Hz, 1H), 7.81 (d, J=9.2 Hz, 1H), 7.33-7.04 (m, 4H), 5.82 (s, 1H), 3.95 (d, J=14.0 Hz, 1H), 2.72-2.51 (m, 3H), 2.36 (s, 3H), 2.15-2.04 (m, 4H).

### Example 13

### 2-(2-([1,2,4]triazolo[1,5-a]pyridin-6-yl)-4-oxo-3-m-tolyl-1-oxaspiro[4.5]dec-2-en-8-ylidene)acetamide

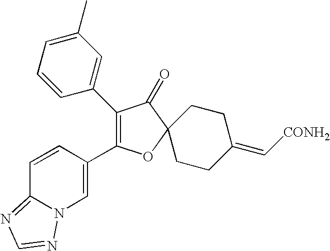

DMF (0.05 mL) was added to a solution of 2-(2-([1,2,4]triazolo[1,5-a]pyridin-6-yl)-4-oxo-3-m-tolyl-1-oxaspiro[4.5]dec-2-en-8-yl)acetic acid (62 mg, 0.15 mmol) in dichloromethane (5 mL). Then (COCl)₂ (57 mg, 0.45 mmol) was added dropwise cautiously at 0° C. and stirred for 1 h. After removal of solvent in vacuo, the residue was dissolved in methylene chloride (5 mL) and ammonia sparged into the mixture at room temperature for 10 min. The mixture was concentrated and the residue extracted with MeOH (2×5 mL). After removal of solvent in vacuo, the residue was purified by HPLC to give the desired product (40 mg, Y: 64%) as a yellow solid.

¹H NMR, (400 MHz, CDCl₃) δ 9.11 (s, 1H), 8.50 (s, 1H), 7.70 (q, J=9.6 Hz, 2H), 7.32-7.06 (m, 4H), 6.21 (s, 1H), 5.76 (s, 1H), 5.60 (s, 1H), 3.94 (d, J=13.6 Hz, 1H), 2.73-2.06 (m, 7H), 2.36 (s, 3H).

MS ESI: 415 (M+1).

### Example 14

### 2-(2-([1,2,4]triazolo[1,5-a]pyridin-6-yl)-4-oxo-3-m-tolyl-1-oxaspiro[4.5]dec-2-en-8-ylidene)-N-(3-(2-oxopyrrolidin-1-yl)propyl)acetamide

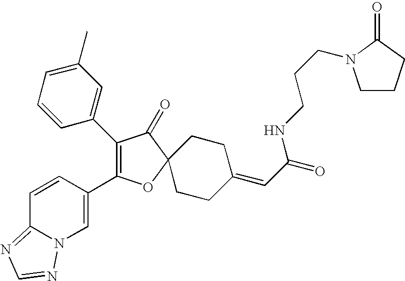

To a solution of 2-(2-([1,2,4]triazolo[1,5-a]pyridin-6-yl)-4-oxo-3-m-tolyl-1-oxaspiro[4.5]dec-2-en-8-yl)acetic acid (62 mg, 0.15 mmol) in dichloromethane (5 mL), HATU (57 mg, 0.15 mmol) and 1-(3-aminopropyl)pyrrolidin-2-one (32 mg, 0.23 mmol) was added and stirred at room temperature for 10 hours. The mixture was concentrated and the residue extracted with MeOH (2×5 mL). After removal of solvent in vacuo, the residue was purified by preparation-HPLC to give the desired product (35 mg, yield 43%) as a yellow solid.

¹H NMR, (400 MHz, CDCl₃) δ 9.09 (s, 1H), 8.49 (s, 1H), 7.69 (q, J=8.5 Hz, 2H), 7.31-7.06 (m, 4H), 6.81 (s, 1H), 5.76 (s, 1H), 4.02 (d, J=13.6 Hz, 1H), 3.42 (m, 4H), 3.27 (m, 2H), 2.73-1.72 (m, 13H), 2.36 (s, 3H).

MS ESI: 540 (M+1).

### Example 15

### 2-([1,2,4]triazolo[1,5-a]pyridin-6-yl)-8-(1,3-dioxolan-2-yl)-3-m-tolyl-1-oxaspiro[4.5]dec-2-en-4-one

### Step 1: 8-(methoxymethylene)-1,4-dioxaspiro[4.5]decane

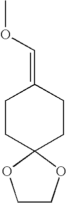

To a suspension of (methoxymethyl)triphenylphosphonium chloride (205 g, 0.60 mol) in 1700 mL of THF was added dropwise n-BuLi (220 mL, 0.55 mol, 2.5 M) at 0° C. The suspension was stirred at 0° C. for 2 hours, then a solution of 1,4-dioxaspiro[4.5]decan-8-one (78 g, 0.5 mol) was added dropwise. The mixture was allowed to warm to room temperature and stirred overnight. The mixture was quenched with saturated NH₄Cl solution (300 mL), and partitioned with ethyl acetate (1000 mL) and H₂O (800 mL). The organic layer was dried over anhydrous Na₂SO₄, and concentrated under reduced pressure. The residue was purified on a silica gel column with 1-5% ethyl acetate/petroleum ether as eluant to give 8-(methoxymethylene)-1,4-dioxaspiro[4.5]decane (52 g, 56%) as a colorless oil.

¹H NMR, (400 MHz, CDCl₃) δ 5.78 (s, 1H), 3.94 (s, 4H), 3.53 (s, 3H), 2.32-2.28 (t, J=6.4 Hz, 2H), 2.10-2.06 (t, J=6.4 Hz, 2H), 1.64-1.60 (m, 4H).

### Step 2: 4-oxocyclohexanecarbaldehyde

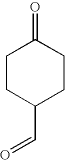

To a solution of 8-(methoxymethylene)-1,4-dioxaspiro[4.5]decane (30 g, 0.16 mol) in 1,4-dioxane (60 mL) was added H₂O (30 mL) and conc. HCl solution (120 mL, 1.4 mol) at room temperature. The mixture was stirred at room temperature for 1 hr. The mixture was partitioned with ethyl acetate (150 mL) and H₂O (150 mL), the organic layer was dried over anhydrous Na₂SO₄, and concentrated under reduced pressure to give a crude 4-oxocyclohexanecarbaldehyde (15.3 g, 86.9%) as a colorless oil, which was used in the next step without further purification.

¹H NMR, (400 MHz, CDCl₃) δ 9.71 (s, 1H), 2.64-2.62 (m, 1H), 2.41-2.29 (m, 5H), 2.20-2.15 (m, 2H), 1.94-1.89 (m, 2H).

### Step 3: 4-(1,3-dioxolan-2-yl)cyclohexanone

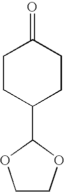

To a solution of 4-oxocyclohexanecarbaldehyde (15.1 g, 120 mmol) and ethylene glycol (7.4 g, 120 mol) in benzene (300 mL) was added p-TsOH (0.21 g, 1.2 mmol) at room temperature. The mixture was heated to reflux and stirred overnight. Benzene was removed under reduced pressure, and the residue was purified on a silica gel column with 5-10% ethyl acetate/petroleum ether as eluant to give 4-(1,3-dioxolan-2-yl)cyclohexanone (9.1 g, 44%) as a colorless oil.

¹H NMR, (400 MHz, CDCl₃) δ 4.73 (d, J=4.4 Hz, 1H), 3.99-3.83 (m, 4H), 2.44-2.24 (m, 4H), 2.13-2.09 (m, 2H), 2.03-1.94 (m, 1H), 1.65-1.53 (m, 2H).

### Step 4: 4-(1,3-dioxolan-2-yl)-1-((trimethylsilyl)ethynyl)cyclohexanol

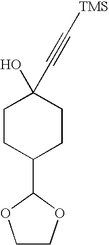

To a solution of ethynyltrimethylsilane (6.23 g, 63.6 mmol) in THF (80 mL) was added n-BuLi (26 mL, 2.5 M, 65 mmol) dropwise at −78° C. under nitrogen. The solution was stirred at −78° C. for 1 hr. Then a solution of 4-(1,3-dioxolan-2-yl)cyclohexanone (9.0 g, 53 mmol) in THF (20 mL) was added dropwise to the stirring solution at this temperature. The mixture was stirred at −78° C. for 1 hr, then allowed to warm to room temperature and stirred overnight. The mixture was quenched with saturated NH₄Cl solution (50 mL), and partitioned with ethyl acetate (200 mL) and H₂O (200 mL). The organic layer was dried over anhydrous Na₂SO₄, filtered and concentrated under reduced pressure to give the desired product (13 g, 91%) as a yellow oil.

¹H NMR, (400 MHz, CDCl₃) δ 4.62 (d, J=12.8 Hz, 1H), 3.95-3.82 (m, 4H), 2.04-1.95 (m, 1H), 1.83-1.66 (m, 3H), 1.53-1.40 (m, 5H), 0.16 (s, 9H).

### Step 5: 4-(1,3-dioxolan-2-yl)-1-ethynylcyclohexanol

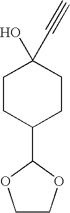

A suspension of 4-(1,3-dioxolan-2-yl)-1-((trimethylsilypethynyl)cyclohexanol (13 g, 48.51 mmol) and K₂CO₃ (20.1 g, 145 mmol) in MeOH (150 mL) was stirred at room temperature for 2 hours. The mixture was filtered and the filtrate concentrated under reduced pressure. The residue was purified on a short silica gel column with 20-50% dichloromethane/ethyl acetate to give 4-(1,3-dioxolan-2-yl)-1-ethynylcyclohexanol (7.1 g, 74%) as a yellow oil.

¹H NMR, (400 MHz, CDCl₃) δ 4.61 (d, J=1.6 Hz, 1H), 3.93-3.78 (m, 4H), 2.59 (s, 1H), 2.02-1.95 (m, 2H), 1.80-1.64 (m, 2H), 1.53-1.39 (m, 5H).

### Step 6: (4-(1,3-dioxolan-2-yl)-1-ethynylcyclohexyloxy)trimethylsilane

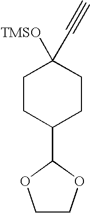

To a solution of 4-(1,3-dioxolan-2-yl)-1-ethynylcyclohexanol (5.3 g, 27 mmol), DMAP (65 mg, 0.54 mmol) and TEA (8.2 g, 81 mmol) in dichloromethane (100 mL) was added TMSCl (4.4 g, 40 mmol) slowly at 0° C. The mixture was allowed to warm to room temperature and stirred for 4 hr. Dichloromethane (200 mL) and H₂O (200 mL) were added and the aqueous phase extracted with dichloromethane (200 mL×2). The combined organic phase was dried over anhydrous Na₂SO₄, filtered and concentrated under reduced pressure. The residue was purified on a short silica gel column with dichloromethane as eluant to give compound (4-(1,3-dioxolan-2-yl)-1-ethynylcyclohexyloxy)trimethylsilane (6.7 g, 92%) as a white solid.

¹H NMR, (400 MHz, CDCl₃) δ 4.61 (d, J=1.6 Hz, 1H), 3.95-3.80 (m, 4H), 2.52 (s, 1H), 2.03-1.96 (m, 2H), 1.77-1.76 (m, 2H), 1.60-1.48 (m, 5H), 0.18 (s, 1H).

### Step 7: 3-(4-(1,3-dioxolan-2-yl)-1-(trimethylsilyloxy)cyclohexyl)-1-([1,2,4]triazolo[1,5-a]pyridin-6-yl)prop-2-yn-1-one

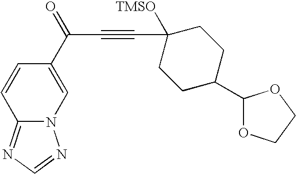

A solution of n-BuLi (16 mL, 2.5 M, 40 mol) was added dropwise to a stirring solution of 4-(1,3-dioxolan-2-yl)-1-ethynylcyclohexyloxy)trimethylsilane (10.3 g, 38 mmol) in THF (80 mL) at −78° C. under nitrogen. The mixture was stirred at −78° C. for 1 hr before adding a solution of N-methoxy-N-methyl-[1,2,4]triazolo[1,5-a]pyridine-6-carboxamide (6.6 g, 32 mmol) in THF (30 mL). After addition, the mixture was allowed to warm to room temperature and stirred overnight. The mixture was quenched with saturated NH₄Cl solution (50 mL), and partitioned with ethyl acetate (100 mL) and H₂O (100 mL). The organic layer was washed with brine (100 mL), dried over anhydrous Na₂SO₄, filtered and concentrated under reduced pressure. The residue was purified on a silica gel column with 10-50% ethyl acetate/petroleum ether as eluant to give 3-(4-(1,3-dioxolan-2-yl)-1-(trimethylsilyloxy)cyclohexyl)-1-([1,2,4]triazolo[1,5-a]pyridin-6-yl)prop-2-yn-1-one (9.1 g, 68%) as a yellow solid.

¹H NMR, (400 MHz, CDCl₃) δ 9.43 (s, 1H), 8.48 (s, 1H), 8.20 (d, J=9.2 Hz, 1H), 7.81 (d, J=9.2 Hz, 1H), 4.76 (d, J=2.8 Hz, 1H), 4.00-3.88 (m, 4H), 2.22 (d, J=11.6 Hz, 2H), 1.90 (d, J=10.8 Hz, 2H), 1.67-1.58 (m, 5H).

### Step 8: 2-([1,2,4]triazolo[1,5-a]pyridin-6-yl)-8-(1,3-dioxolan-2-yl)-1-oxaspiro[4.5]dec-2-en-4-one

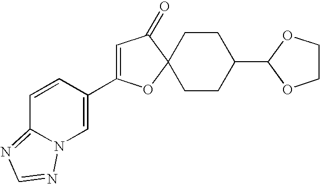

A mixture of 3-(4-(1,3-dioxolan-2-yl)-1-(trimethylsilyloxy)cyclohexyl)-1-([1,2,4]triazolo[1,5-a]pyridin-6-yl)prop-2-yn-1-one (6.1 g, 14 mmol) and K₂CO₃ (6.1 g, 44 mmol) in MeOH (100 mL) was stirred at room temperature for 2 h. Diethylamine (2.69 g, 36.9 mmol) was added at room temperature and the mixture stirred overnight. The resulting mixture was filtered and the filtrate was concentrated under reduced pressure. The residue was purified on a silica gel column using 10% methylene chloride/ethyl acetate as eluant to give 2-([1,2,4]triazolo[1,5-a]pyridin-6-yl)-8-(1,3-dioxolan-2-yl)-1-oxaspiro[4.5]dec-2-en-4-one (4.57 g, 90.8%).

¹H NMR, (400 MHz, CDCl₃) δ 8.75 (s, 1H), 8.33 (s, 1H), 7.73-7.64 (m, 2H), 4.80-4.77 (m, 2H), 4.08-3.86 (m, 4H), 2.13-1.73 (m, 9H).

### Step 9: 2-([1,2,4]triazolo[1,5-a]pyridin-6-yl)-8-(1,3-dioxolan-2-yl)-3-m-tolyl-1-oxaspiro[4.5]dec-2-en-4-one

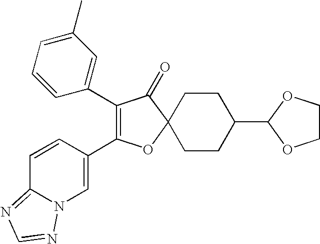

To a 100 mL flask CsOAc (5.07 g, 26.4 mmol) was added and heated under vacuum at 120° C. for 2 hours. After cooling to room temperature, Pd(OAc)₂ (99 mg, 0.44 mmol), (p-PhCF₃)₃P (205 mg, 0.44 mmol) and anhydrous DMF (50 mL) were added and the mixture stirred for 30 min at room temperature. A mixture of 2-([1,2,4]triazolo[1,5-a]pyridin-6-yl)-8-(1,3-dioxolan-2-yl)-1-oxaspiro[4.5]dec-2-en-4-one (3.0 g, 8.8 mmol) and 1-iodo-3-methylbenzene (2.3 g, 10.6 mmol) was added and the mixture stirred for 10 min then heated at 120° C. overnight. The mixture was cooled to room temperature and diluted with ethyl acetate (150 mL) and water (500 mL). The aqueous phase was extracted with ethyl acetate (30 mL×3), the combined organic layers dried over MgSO₄, concentrated and the residue purified on a silica gel column using 1:1 (v/v) ethyl acetate/petroleum ether as eluant to give 2-([1,2,4]biazolo[1,5-a]pyridin-6-yl)-8-(1,3-dioxolan-2-yl)-3-m-tolyl-1-oxaspiro[4.5]dec-2-en-4-one (760 mg, yield 21%) as a yellow solid. ¹H NMR, (400 MHz, CDCl₃) δ 9.09 (s, 1H), 8.52 (s, 1H), 7.88 (d, J=9.6 Hz, 1H), 7.77 (d, J=9.2 Hz, 1H), 7.32-7.28 (t, J=7.6 Hz, 1H), 7.20 (d, J=8.0 Hz, 1H), 7.13 (s, 1H), 7.04 (d, J=7.2 Hz, 1H), 4.87 (d, J=5.6 Hz, 1H), 4.02-3.89 (m, 4H), 2.35 (s, 3H), 2.13-2.03 (m, 4H), 1.96-1.81 (m, 5H).

MS ESI: 432 (M+1).

### Example 16

### 2-([1,2,4]triazolo[1,5-a]pyridin-6-yl)-8-((pyridin-3-ylamino)methyl)-3-m-tolyl-1-oxaspiro[4.5]dec-2-en-4-one

### Step 1: 2-([1,2,4]triazolo[1,5-a]pyridin-6-yl)-4-oxo-3-m-tolyl-1-oxaspiro[4.5]dec-2-ene-8-carbaldehyde

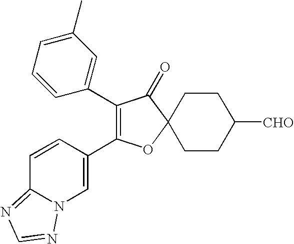

To a solution of the starting material 1 (50 mg, 0.12 mmol) in 1,4-dioxane (2 mL) was added 0.4 mL of H₂O and 0.7 mL of concentrated HCl dropwise at room temperature. The reaction mixture was stirred at r.t. for 1 hour. After the reaction was complete, the resulting mixture was partitioned with ethyl acetate and water. The aqueous phase was extracted with ethyl acetate, and the combined organic layer was dried over anhydrous Na₂SO₄, filtered, and concentrated under reduced pressure to give the crude product 2, which was used for the next step without further purification.

¹H NMR (CDCl₃, 400 MHz) δ 9.76 (s, 1H), 9.05 (s, 1H), 8.42 (s, 1H), 7.69-7.62 (m, 2H), 7.31 (t, J=7.6 Hz, 1H), 7.20 (d, J=7.1 Hz, 1H), 7.14 (s, 1H), 7.06 (d, J=7.6 Hz, 1H), 2.55-2.49 (m, 1H), 2.38 (s, 3H), 2.20-2.17 (m, 2H), 1.97-1.95 (m, 4H), 1.84-1.78 (m, 2H).

MS (m+1): 388.2.

### Step 2: 2-([1,2,4]triazolo[1,5-a]pyridin-6-yl)-8-((pyridin-3-ylamino)methyl)-3-m-tolyl-1-oxaspiro[4.5]dec-2-en-4-one

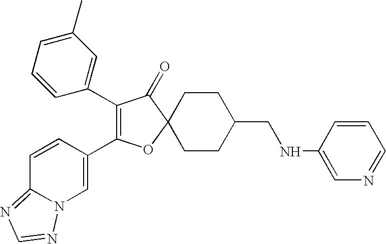

To a solution of 2-([1,2,4]triazolo[1,5-a]pyridin-6-yl)-4-oxo-3-m-tolyl-1-oxaspiro[4.5]dec-2-ene-8-carbaldehyde (128 mg, 0.33 mmol) in EtOH (4 mL) was added pyridin-3-ylmethanamine (39.3 mg, 0.364 mmol) at room temperature. The mixture was stirred at room temperature for 2 days. After the reaction completion, NaBH(OAc)₃ (280 mg, 1.32 mmol) was added to the solution with stirring. The reaction mixture was stirred at room temperature overnight. The mixture was partitioned with CH₂Cl₂ (10 mL) and H₂O (50 mL), and the aqueous phase extracted with CH₂Cl₂ (10 mL*3). The combined organic layer was dried over anhydrous Na₂SO₄, filtered and concentrated under reduced pressure. The residue was purified by preparative- HPLC to give 2-([1,2,4]triazolo[1,5-a]pyridin-6-yl)-8-((pyridin-3-ylamino)methyl)-3-m-tolyl-1-oxaspiro[4.5]dec-2-en-4-one (50 mg, 31%).

¹H NMR, (400 MHz, CDCl₃) δ 9.11 (s, 1H), 9.00 (d, J=8.8 Hz, 1H), 8.65 (s, 1H), 8.44-8.43 (m, 2H), 7.74-7.59 (m, 3H), 7.28-7.26 (m, 1H), 7.20 (d, J=5.6 Hz, 1H), 7.07 (s, 1H), 7.00 (d, J=10.0 Hz, 1H), 4.42 (s, 2H), 3.09 (d, J=1.6 Hz, 2H), 2.31 (s, 3H), 2.11-1.50 (m, 9H). MS ESI: 466 (M+1).

### Example 17

### 2-([1,2,4]triazolo[1,5-a]pyridin-6-yl)-8-(1,3-dioxolan-2-yl)-3-(4-fluoro-3-methylphenyl)-1-oxaspiro[4.5]dec-2-en-4-one

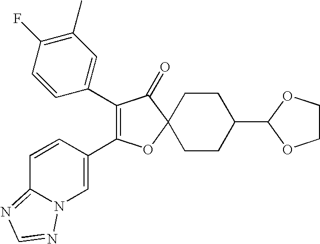

The title compound was prepared according to the procedures of Example 15 and substituting appropriate starting materials.

¹H NMR, (400 MHz, CDCl₃) δ 9.10 (s, 1H), 8.52 (s, 1H), 7.86 (d, J=9.6 Hz, 1H), 7.75 (d, J=8.4 Hz, 1H), 7.16 (d, J=7.6 Hz, 1H), 7.06 (d, J=6.4 Hz, 2H), 4.86 (d, J=5.6 Hz, 1H), 4.02-3.89 (m, 4H), 2.35 (s, 3H), 2.12-2.03 (m, 4H), 1.94-1.80 (m, 5H). MS ESI: 450 (M+1).

### Example 18

### 2-([1,2,4]triazolo[1,5-a]pyridin-6-yl)-3-(4-fluoro-3-methylphenyl)-4-oxo-1-oxaspiro[4.5]dec-2-ene-8-carboxylic acid

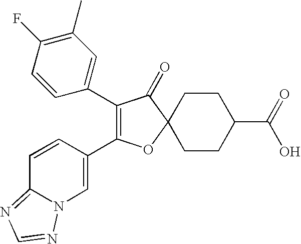

### Step 1: 2-([1,2,4]triazolo[1,5-a]pyridin-6-yl)-3-(4-fluoro-3-methylphenyl)-4-oxo-1-oxaspiro[4.5]dec-2-ene-8-carbaldehyde

The title compound was prepared according to the procedures of Example 16, step 1 MS (M+1): 406.2.

### Step 2: 2-([1,2,4]triazolo[1,5-a]pyridin-6-yl)-3-(4-fluoro-3-methylphenyl)-4-oxo-1-oxaspiro[4.5]dec-2-ene-8-carboxylic acid

To a solution of 2-([1,2,4]triazolo[1,5-a]pyridin-6-yl)-3-(4-fluoro-3-methylphenyl)-4-oxo-1-oxaspiro[4.5]dec-2-ene-8-carbaldehyde (0.83 g, 2.0 mmol) in acetone (10 mL) was added Jones' reagent (30 mL, 90 mmol) at room temperature. The mixture was stirred at room temperature overnight then partitioned with CH₂Cl₂ (30 mL) and H₂O (50 mL), the aqueous phase extracted with CH₂Cl₂ (20 mL×2) and the combined organic layer dried over anhydrous Na₂SO₄, filtered and concentrated under reduced pressure. The residue was purified by preparation-HPLC to give 2-([1,2,4]triazolo[1,5-a]pyridin-6-yl)-3-(4-fluoro-3-methylphenyl)-4-oxo-1-oxaspiro[4.5]dec-2-ene-8-carboxylic acid (80 mg, yield 10%) as a yellow solid.

¹H NMR, (400 MHz, CDCl₃) δ 9.19 (s, 1H), 8.49 (s, 1H), 7.82-7.67 (m, 2H), 7.16 (d, J=9.2

Hz, 1H), 7.07-7.05 (d, J=10.0 Hz, 2H), 2.33-2.18 (m, 4H), 2.04-1.96 (m, 8H).

MS (M+1): 422.

### Example 19

### 2-([1,2,4]triazolo[1,5-a]pyridin-6-yl)-3-(4-fluoro-3-methylphenyl)-4-oxo-1-oxaspiro[4.5]dec-2-ene-8-carboxamide.

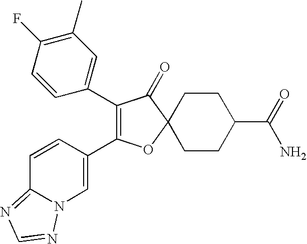

To a solution of 2-([1,2,4]triazolo[1,5-a]pyridin-6-yl)-3-(4-fluoro-3-methylphenyl)-4-oxo-1-oxaspiro[4.5]dec-2-ene-8-carboxylic acid (25 mg, 0.059 mmol) in dichloromethane (4 mL), was added DMF (0.04 mL). Then (COCl)₂ (22.43 mg, 0.18 mmol) was added dropwise cautiously at 0° C. and the resultant mixture stirred for 1 h. After removal of solvent in vacuo, the residue was dissolved in methylene chloride (5 mL) and the mixture sparged with ammonia at room temperature for 10 min. The mixture was concentrated and the residue extracted with MeOH (5 mL×2). After removal of the solvent in vacuo, the residue was purified by preparative HPLC to give 2-([1,2,4]triazolo[1,5-a]pyridin-6-yl)-3-(4-fluoro-3-methylphenyl)-4-oxo-1-oxaspiro[4.5]dec-2-ene-8-carboxamide (7.0 mg, Y: 28%) as a yellow solid.

¹H NMR, (400 MHz, CDCl₃) δ 9.09 (s, 1H), 8.44 (s, 1H), 7.74-7.67 (m, 2H), 7.16 (d, J=6.8 Hz, 1H), 7.07-7.03 (m, 2H), 6.02-5.68 (m, 2H), 2.48-2.32 (m, 1H), 2.32 (s, 3H), 2.16-1.82 (m, 8H). MS ESI: 421 (M+1).

### Example 20

### 5-([1,2,4]triazolo[1,5-a]pyridin-6-yl)-2,2-dimethyl-4-m-tolylfuran-3(2H)-one

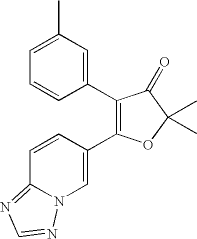

Cesium acetate (100 mg, 0.000523 mol) was added a round bottom flask and dried under vacuum at 125° C. for 2 hours. Palladium acetate (0.588 mg, 2.62E-6 mol), tris(4-trifluoromethylphenyl)phosphine (4.88 mg, 0.0000105 mol), and N,N-dimethylformamide (1.0 mL, 0.013 mol) were added to the dried cesium acetate and the mixture stirred for 30 min. In a separate flask, 5-([1,2,4]triazolo[1,5-a]pyridin-6-yl)-2,2-dimethylfuran-3(2H)-one (60.0 mg, 0.000262 mol), and 1-iodo-3-methylbenzene (0.0335 mL, 0.000262 mol) were added to N,N-dimethylformamide (1.0 mL, 0.013 mol). The second solution was added to the first solution. The mixture was heated at 125° C. for 18 hours. The cooled mixture was partitioned between ethyl acetate and water. The organic phase was washed with brine, dried over sodium sulfate, filtered, and concentrated to give a yellow oil. The yellow oil was purified on HPLC eluting with acetonitrile:water (0.1% TFA) to give 30 mg (40%) of the title compound as a yellow oil. ¹H NMR (300 MHz, CDCl₃) δ 9.07 (s, 1H), 8.46 (s, 1H), 7.73 (m, 2H), 7.29 (m, 1H), 7.20 (d, 1H, J=6.0 Hz), 7.13 (s, 1H), 7.06 (d, 1H, J=6.0 Hz), 2.35 (s, 3H), 1.59 (s, 6H). MS (ESP⁺) m/z 320.33.

### Example 21

### 5-([1,2,4]triazolo[1,5-a]pyridin-6-yl)-4-(3-chlorophenyl)-2,2-dimethylfuran-3(2H)-one

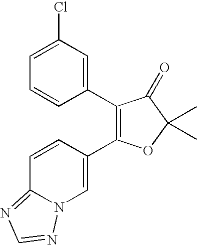

Cesium acetate (167 mg, 0.000872 mol) was added a round bottom flask and dried under vacuum at 125° C. for 2 hours. Palladium acetate (0.98 mg, 0.0000044 mol), tris(4-trifluoromethylphenyl)phosphine (8.1 mg, 0.000017 mol), and N,N-dimethylformamide (1.7 mL, 0.022 mol) were added to the dried cesium acetate and the mixture stirred for 30 min. In a separate flask, 5-([1,2,4]triazolo[1,5-a]pyridin-6-yl)-2,2-dimethylfuran-3(2H)-one (100.0 mg, 0.0004362 mol), and 1-chloro-3-iodobenzene, (0.05402 mL, 0.0004363 mol) were added to N,N-dimethylformamide (1.69 mL, 0.0218 mol). The second solution was added to the first solution. The mixture was heated at 125° C. for 18 hours. The mixture was then partitioned between ethyl acetate and water. The organic phase was washed with brine, dried over sodium sulfate, filtered, and concentrated to give a yellow oil. The yellow oil was purified on HPLC eluting with acetonitrile:water (0.1% TFA) to give 5.0 mg (3.4%) of the title compound as a yellow solid. ¹H NMR (300 MHz, Methanol-d₄) δ 9.17 (s, 1H), 8.52 (s, 1H), 7.73 (m, 2H), 7.40 (m, 3H), 7.24 (m, 1H), 1.59 (s, 6H). MS (ESP⁺) m/z 340.11.

### Example 22

### 5-([1,2,4]triazolo[1,5-a]pyridin-6-yl)-4-(3-chloro-4-fluorophenyl)-2,2-dimethylfuran-3(2H)-one

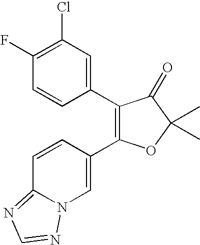

Cesium acetate (167 mg, 0.000872 mol) was added to a round bottom flask and dried under vacuum at 125° C. for 2 hours. Palladium acetate (0.98 mg, 0.0000044 mol), tris(4-trifluoromethylphenyl)phosphine (8.1 mg, 0.000017 mol), and N,N-dimethylformamide (1.7 mL, 0.022 mol) were added to the dried cesium acetate and the mixture stirred for 30 min. 5-([1,2,4]triazolo[1,5-a]pyridin-6-yl)-2,2-dimethylfuran-3(2H)-one (100.0 mg, 0.0004362 mol) and 2-chloro-1-fluoro-4-iodobenzene (112 mg, 0.000436 mol) in N,N-dimethylformamide (1.7 mL, 0.022 mol) were added and the mixture stirred at 125° C. for 18 hr. The mixture was partitioned between ethyl acetate and water. The organic phase was washed with brine, dried over sodium sulfate, filtered, and concentrated to give a yellow oil. The yellow oil was purified on HPLC eluting with acetonitrile:water (0.1% TFA) to give 20.0 mg (12.8%) of the title compound as a yellow solid.

¹H NMR (300 MHz, Methanol-d₄) δ 9.19 (s, 1H), 8.49 (s, 1H), 7.75 (m, 3H), 7.29 (m, 2H), 1.59 (s, 6H). MS (ESP⁺) m/z 358.09.

### Example 23

### 3-(benzo[d][1,3]dioxol-5-yl)-2-(pyridin-4-yl)-1-oxa-8-azaspiro[4.5]dec-2-en-4-one

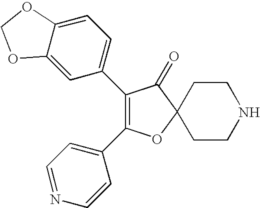

### Step 1: Tert-butyl 4-hydroxy-4-((trimethylsilyl)ethynyl)piperidine-1-carboxylate

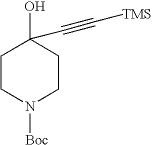

At −78° C., to solution of trimethylsilylacetylene (10 mL, 0.071 mol) in anhydrous THF (100 mL) was added dropwise n-butyllithium in hexane (1.6M, 35 mL) and stirred for 1 hour. A solution of tert-butyl 4-oxopiperidine-1-carboxylate (9.4 g, 0.047 mol) in anhydrous THF (10 mL) was added to the above solution in 10 minutes and stirred at −78° C. for 1 hour then warmed to room temperature and stirred for 3 hr. The mixture was quenched with saturated ammonium chloride and extracted with diethyl ether. The organic phase was washed with water and dried over MgSO₄. Concentration gave a crystalline solid which was used in Step 2 without further purification.

### Step 2: Tert-butyl 4-ethynyl-4-hydroxypiperidine-1-carboxylate

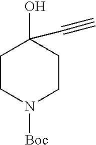

A solution of tert-butyl 4-hydroxy-4-((trimethylsilyl)ethynyl)piperidine-1-carboxylate (14 g, 0.047 mol) in THF (100 mL) was added a solution of 1M TBAF in THF (47 mL) and stirred for 1 h at room temperature. The mixture was concentrated and the residue purified on silica gel column with 100% hexane, then 100% methylene chloride, and 100% ethyl acetate to give the desired product (10.8 g).

¹H NMR (400 MHz, CDCl₃) δ 3.88 (s, 1H), 3.62 (br, 2H), 3.14 (m, 1H), 2.41 (s, 1H), 1.75 (m, 2H), 1.62 (m, 2H) 1.32 (s, 9H)

### Step 3: Tert-butyl 4-ethynyl-4-(trimethylsilyloxy)piperidine-1-carboxylate

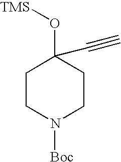

To a solution of tert-butyl 4-ethynyl-4-hydroxypiperidine-1-carboxylate (10.8 g, 0.048 mol) and triethylamine (21 mL, 0.093 mol), and catalytic amount of DMAP in methylene chloride was added 1M TMSCl in methylene chloride (48 mL, 0.048 mol) and the mixture stirred overnight. The mixture was partitioned with methylene chloride and water, the organic phase dried over MgSO₄ and concentrated. The residue was purified on silica gel column with 5% ethyl acetate in hexane to give the desired product as a colorless syrup (10.9 g, 75%)

¹H NMR (400 MHz, CDCl₃) δ 3.62 (br, 2H), 3.20 (m, 1H), 2.50 (s, 1H), 1.82 (m, 2H), 1.70 (m, 2H), 1.42 (s, 9H), 0.1 (s, 9H).

### Step 4: Tert-butyl 4-(3-oxo-3-(pyridin-4-yl)prop-1-ynyl)-4-(trimethylsilyloxy)piperidine-1-carboxylate

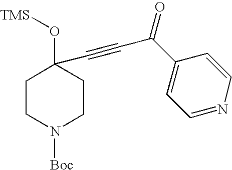

At −78° C., n-butyllithium (1.6M/hexane, 10 mL, 0.016 mol) was added to a solution of tert-butyl 4-ethynyl-4-(trimethylsilyloxy)piperidine-1-carboxylate (5.1 g, 0.017 mol) and the mixture stirred for 1 h. A solution of N-methoxy-N-methylisonicotinamide (3.4 g, 0.020 mol) was added and the mixture stirred at room temperature for 3 h. The mixture was quenched with saturated ammonium chloride and extracted with diethyl ether. The organic phase was washed with water, dried over MgSO₄ and concentrated. The residue was purified on silica gel column with 5-10% ethyl acetate in methylene chloride to give the desired product (4.1 g, 59%)

¹H NMR (300 MHz, CDCl₃) δ 8.85 (d, 2H), 7.85 (d, 2H), 3.65 (m, 2H), 3.40 (m, 2H), 2.0 (m, 2H), 1.85 (m, 2H), 1.45 (s, 9H), 0.2 (s, 9H),

### Step 5: Tert-butyl 4-oxo-2-(pyridin-4-yl)-1-oxa-8-azaspiro[4.5]dec-2-ene-8-carboxylate

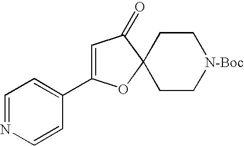

A solution of tert-butyl 4-(3-oxo-3-(pyridin-4-yl)prop-1-ynyl)-4-(trimethylsilyloxy)piperidine-1-carboxylate (4.1 g, 0.01 mol) and TsOH monohydrate (1.9 g, 0.01 mol) in methanol was stirred for 1 h then concentrated. The residue was partitioned between saturated sodium bicarbonate and ethyl acetate. The organic phase was dried over MgSO₄ and concentrated to give a crude product as a dark brown foam, which was then mixed with diethylamine (1.13 mL, 0.012 mol) in ethanol (100 mL) for 15 minutes before it was concentrated. The residue was stirred with a solution of 0.1% TFA in water and acetonitrile (1:1) for 1 h and partitioned between ethyl acetate and sat. NaHCO₃. The organic phase was dried over MgSO₄, concentrated and the residue purified on a silica gel column with 75% ethyl acetate in methylene chloride to give the title compound (1.9 g, 57%).

¹H NMR (300 MHz, CDCl₃) δ 8.9 (d, 2H), 8.0 (d, 2H), 6.3 (s, 1H), 4.20 (m, 2H), 3.25 (m, 2H), 2.0 (m, 2H), 1.85 (m, 2H), 1.45 (s, 9H)

MS (ESP⁺) m/z 331.27

### Step 6: Tert-butyl 3-bromo-4-oxo-2-(pyridin-4-yl)-1-oxa-8-azaspiro[4.5]dec-2-ene-8-carboxylate

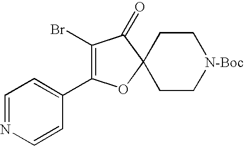

A solution of tert-butyl 4-oxo-2-(pyridin-4-yl)-1-oxa-8-azaspiro[4.5]dec-2-ene-8-carboxylate (266 mg, 0.81 mmol) and N-bromosuccinamide (143 mg, 0.81 mmol) in anhydrous chloroform (50 mL) was stirred at room temperature overnight. Another portion of NBS (100 mg, 0.56 mmol) was added and the mixture heated to 60° C. for 10 min then concentrated. The residue was purified on a silica gel column with 40% ethyl acetate in methylene chloride to give the title compound as an off-white solid (270 mg, 82%)

¹H NMR (400 MHz, CD₃OD) δ 8.8 (d, 2H), 8.2 (d, 2H), 4.20 (m, 2H), 3.30 (m, 2H), 1.90 (m, 2H), 1.80 (m, 2H), 1.50 (s, 9H)

MS (ESP) m/z 409.20/411.20

### Step 7: Tert-butyl 3-(benzo[d][1,3]dioxol-5-yl)-4-oxo-2-(pyridin-4-yl)-1-oxa-8-azaspiro[4.5]dec-2-ene-8-carboxylate

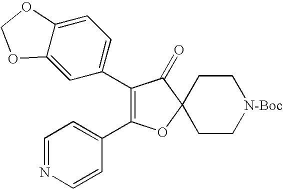

Tert-butyl 3-bromo-4-oxo-2-(pyridin-4-yl)-1-oxa-8-azaspiro[4.5]dec-2-ene-8-carboxylate (1.24 g, 3.0 mmol) and benzo[d][1,3]dioxol-5-ylboronic acid (604 mg, 3.6 mmol) were dissolved in DMF (20 mL). 2M aqueous Na₂CO₃ (7.5 mL, 15.0 mmol) and PdCl₂(dppf) dichloromethane complex (74 mg, 0.09 mmol) was added and the mixture heated to 100° C. for 3 h then cooled to room temperature. The mixture was partitioned between ethyl acetate and water, the organic phase dried over MgSO₄ and concentrated and the residue purified on a silica gel column to give the title compound (420 mg, 31%).

¹H NMR (400 MHz, CDCl₃) δ 8.80 (d, 2H), 7.80 (d, 2H), 6.85 (d, 1H), 6.70 (m, 2H), 4.20 (m, 2H), 3.30 (m, 2H), 1.90 (m, 2H), 1.80 (m, 2H), 1.50 (s, 9H)

MS (ESP⁺) m/z 451.2

### Step 8: 3-(benzo[d][1,3]dioxol-5-yl)-2-(pyridin-4-yl)-1-oxa-8-azaspiro[4.5]dec-2-en-4-one

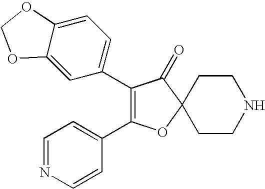

A solution of tert-butyl 3-(benzo[d][1,3]dioxol-5-yl)-4-oxo-2-(pyridin-4-yl)-1-oxa-8-azaspiro[4.5]dec-2-ene-8-carboxylate (420 mg, 0.9 mmol) in trifluoroacetic acid (1 mL) and methylene chloride (5 mL) was stirred at room temperature for 1 h and then concentrated to give the desired product as a TFA salt.

¹H NMR (400 MHz, CDCl₃) δ 8.80 (d, 2H), 7.80 (d, 2H), 6.85 (d, 1H), 6.70 (m, 2H), 3.7 (m, 2H), 3.60 (m, 2H), 2.3-2.1 (m, 4H)

MS (ESP⁺) m/z 351.1

### Example 24

### benzyl 3-(benzo[d][1,3]dioxol-5-yl)-4-oxo-2-(pyridin-4-yl)-1-oxa-8-azaspiro[4.5]dec-2-ene-8-carboxylate

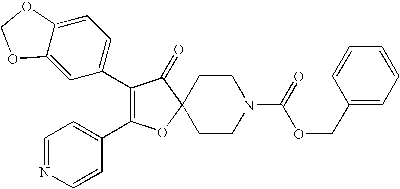

To a solution of 3-(benzo[d][1,3]dioxol-5-yl)-2-(pyridin-4-yl)-1-oxa-8-azaspiro[4.5]dec-2-en-4-one, TFA salt (60 mg) and 2M Na₂CO₃ (0.4 mL) in acetonitrile (5 mL) was added benzyl chloroformate (60 uL) and the mixture stirred for 1 hour. The mixture was diluted with water, extracted with methylene chloride, the organic phase dried over sodium sulfate and concentrated. The residue was purified on HPLC to give the title compound (10 mg).

¹H NMR (400 MHz, CDCl₃) δ 8.80 (d, 2H), 7.80 (d, 2H), 7.40 (m, 5H), 6.80 (d, 1H), 6.70 (m, 2H), 6.0 (s, 2H), 5.20 (s, 2H), 4.25 (s, br, 2H), 3.35 (s, br, 2H), 2.1 (m, 2H), 1.8 (m, 2H)

MS (ESP⁺) m/z 485.2

### Example 25

### 4-(3-(benzo[d][1,3]dioxol-5-yl)-4-oxo-2-(pyridin-4-yl)-1-oxa-8-azaspiro[4,5]dec-2-enecarbonyl)benzonitrile

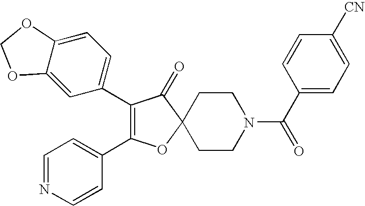

4-Cyanobenzoyl chloride (95 mg) was added to a mixture of 3-(benzo[d][1,3]dioxol-5-yl)-2-(pyridin-4-yl)-1-oxa-8-azaspiro[4.5]dec-2-en-4-one, TFA salt (80 mg) and 5N NaOH (230 uL) in acetonitrile (5 mL) and stirred for 10 minutes. The mixture was diluted with water, extracted with methylene chloride, the organic phase dried over sodium sulfate and concentrated. The residue was purified on HPLC to give the title compound (10 mg).

¹H NMR (400 MHz, CDCl₃) δ 8.80 (d, 2H), 7.80 (d, 2H), 7.40 (m, 5H), 6.80 (d, 1H), 6.70 (m, 2H), 6.0 (s, 2H), 5.20 (s, 2H), 4.25 (s, br, 2H), 3.35 (s, br, 2H), 2.1 (m, 2H), 1.8 (m, 2H)

MS (ESP⁺) m/z 485.2

Additional examples of compounds prepared by the methods described above are provided in Table 1.

TABLE 1
ADDITIONAL EXMPLES OF COMPOUNDS OF FORMUAL (I)

| Example No. | Name | Physical Data |
|---|---|---|
| 26 | tert-butyl 2-(benzo[d][1,3]dioxol-5- | ¹H NMR (400 MHz, CDCl₃) δ 7.85 (s, |
| | yl)-4-oxo-3-(pyridin-3-yl)-1-oxa-8- | 2H), 7.55 (d, 1H), 7.20 (t, 1H), 7.10 (d, |
| | azaspiro[4.5]dec-2-ene-8- | 1H), 6.90 (s, 1H), 6.65 (d, 1H), 5.90 (s, |
| | carboxylate | 2H), 4.15 (br, 2H), 3.14 (br, 1H), 1.90 (m, |
| | | 2H), 1.62 (m, 2H), 1.32 (s, 9H) |
| | | MS (ESP⁺) m/z 451.02 |
| 27 | tert-butyl 2-(benzo[d][1,3]dioxol-5- | ¹H NMR (400 MHz, CDCl₃) δ 8.65 (d, |
| | yl)-4-oxo-3-(pyridin-4-yl)-1-oxa-8- | 2H), 7.30 (d, 2H), 7.25 (d, 1H), 7.10 (d, |
| | azaspiro[4.5]dec-2-ene-8- | 1H), 7.12 (s, 1H), 6.80 (d, 1H), 6.05 (s, |
| | carboxylate | 2H), 4.15 (br, 2H), 3.20 (br, 1H), 2.10 (m, |
| | | 2H), 1.75 (m, 2H), 1.50 (s, 9H) |
| | | MS (ESP⁺) m/z 451.02 |
| 28 | 2-(benzo[d][1,3]dioxol-5-yl)-3- | ¹H NMR (400 MHz, CD₃OD) δ 8.80 (s, |
| | (pyridin-3-yl)-1-oxa-8- | 1H), 8.75 (d, 1H), 8.40 (d, 1H), 7.95 (t, |
| | azaspiro[4.5]dec-2-en-4-one | 2H), 7.37 (d, 1H), 7.29 (s, 1H), 6.95 (d, |
| | | 1H), 6.15 (s, 2H), 3.65 (m, 2H), 3.50 (br, |
| | | 1H), 2.30 (m, 2H), 2.20 (m, 2H), |
| | | MS (ESP⁺) m/z 351.1 |
| 29 | benzyl 2-(benzo[d][1,3]dioxol-5-yl)- | ¹H NMR (400 MHz, CDCl₃) δ 8.72 (d, |
| | 4-oxo-3-(pyridin-4-yl)-1-oxa-8- | 2H), 7.80 (d, 2H), 7.30 (m, 5H), 7.15 (d, |
| | azaspiro[4.5]dec-2-ene-8- | 1H), 6.95 (s, 1H), 6.85 (d, 1H), 6.05 (s, |
| | carboxylate | 2H), 5.05 (s, 2H), 4.20 (br, 2H), 3.25 (br, |
| | | 1H), 2.00 (m, 2H), 1.70 (m, 2H), |
| | | MS (ESP⁺) m/z 485.07 |
| 30 | benzyl 2-(benzo[d][1,3]dioxol-5-yl)- | ¹H NMR (400 MHz, CDCl₃) δ 8.80 (s, |
| | 4-oxo-3-(pyridin-3-yl)-1-oxa-8- | 1H), 8.75 (d, 1H), 8.40 (d, 1H), 7.80 (t, |
| | azaspiro[4.5]dec-2-ene-8- | 2H), 7.37 (m, 5H), 7.15 (d, 1H), 7.00 (s, |
| | carboxylate | 1H), 6.85 (d, 1H), 6.05 (s, 2H), 5.15 (s, |
| | | 2H), 4.20 (br, 2H), 3.30 (br, 1H), 2.05 (m, |
| | | 2H), 1.75 (m, 2H), |
| | | MS (ESP⁺) m/z 485.06 |
| 31 | 4-(2-(benzo[d][1,3]dioxol-5-yl)-4- | ¹H NMR (400 MHz, CDCl₃) δ 8.80 (s, |
| | oxo-3-(pyridin-3-yl)-1-oxa-8- | 1H), 8.75 (d, 1H), 8.25 (d, 1H), 7.80 (t, |
| | azaspiro[4.5]dec-2- | 1H), 7.70 (d, 2H), 7.50 (d, 2H), 7.15 (d, |
| | enecarbonyl)benzonitrile | 1H), 7.00 (s, 1H), 6.85 (d, 1H), 6.05 (s, |
| | | 2H), 5.15 (s, 2H), 4.20 (br, 2H), 3.30 (br, |
| | | 1H), 2.05 (m, 2H), 1.75 (m, 2H), |
| | | MS (ESP⁺) m/z 480.02 |
| 32 | 2-(benzo[d][1,3]dioxol-5-yl)-3- | ¹H NMR (400 MHz, CDCl₃) δ 8.80 (s, |
| | (pyridin-3-yl)-8-tosyl-1-oxa-8- | 1H), 8.75 (d, 1H), 8.25 (d, 1H), 7.80 (t, |
| | azaspiro[4.5]dec-2-en-4-one | 1H), 7.70 (d, 2H), 7.40 (d, 2H), 7.05 (d, |
| | | 1H), 6.90 (s, 1H), 6.80 (d, 1H), 6.05 (s, |
| | | 2H), 3.85 (d, 2H), 2.95 (bt, 2H), 2.50 (s, |
| | | 3H), 2.20 (dt, 2H), 1.90 (d, 2H), |
| | | MS (ESP⁺) m/z 505.2 |
| 33 | tert-butyl 3-(benzo[d][1,3]dioxol-5- | ¹H NMR (400 MHz, CDCl₃) δ 8.80 (d, |
| | yl)-4-oxo-2-(pyridin-2-yl)-1-oxa-8- | 1H), 7.80 (t, 2H), 7.65 (d, 1H), 7.50 (dt, |
| | azaspiro[4.5]dec-2-ene-8- | 1H), 6.8-6.75 (m, 3H) 5.90 (s, 2H), 4.15 |
| | carboxylate | (br, 2H), 3.20 (br, 1H), 2.00 (dt, 2H), 1.75 |
| | | (d, 2H), 1.50 (s, 9H) |
| | | MS (ESP⁺) m/z 451.2 |
| 34 | 3-(benzo[d][1,3]dioxol-5-yl)-2- | ¹H NMR (400 MHz, CD₃OD) δ 8.80 (d, |
| | (pyridin-2-yl)-1-oxa-8- | 1H), 7.80 (t, 2H), 7.65 (d, 1H), 7.50 (dt, |
| | azaspiro[4.5]dec-2-en-4-one | 1H), 6.8 (s, 1H), 6.75 (m, 2H) 6.00 (s, |
| | | 2H), 3.6-3.5 (m, 4H), 2.25 (dt, 2H), 2.10 |
| | | (d, 2H) |
| | | MS (ESP⁺) m/z 351.1 |
| 35 | benzyl 3-(benzo[d][1,3]dioxol-5-yl)- | ¹H NMR (400 MHz, CDCl₃) δ 8.90 (d, |
| | 4-oxo-2-(pyridin-2-yl)-1-oxa-8- | 1H), 7.80 (t, 2H), 7.65 (d, 1H), 7.50 (dt, |
| | azaspiro[4.5]dec-2-ene-8- | 1H), 7.35 (m, 5H), 6.8 (s, 1H), 6.75 (m, |
| | carboxylate | 2H) 6.00 (s, 2H), 5.15 (s, 2H), 4.2 (br, |
| | | 2H), 3.40 (br, 2H), 2.05(br, 2H), 1.80 (br, |
| | | 2H) |
| | | MS (ESP⁺) m/z 485.2 |
| 36 | 4-(3-(benzo[d][1,3]dioxol-5-yl)-4- | ¹H NMR (400 MHz, CDCl₃) δ 8.95 (s, |
| | oxo-2-(pyridin-2-yl)-1-oxa-8- | 1H), 7.85 (t, 1H), 7.75 (d, 2H), 7.65 (d, |
| | azaspiro[4.5]dec-2- | 1H), 7.60 (dt, 1H), 7.50 (d, 2H), 6.85 (d, |
| | enecarbonyl)benzonitrile | 1H), 6.75, s, 1H), 6.74 (s, 1H), 6.05 (s, |
| | | 2H), 3.7 (br, 2H), 3.50 (br, 1H), 2.05 (m, |
| | | 2H), 1.75 (m, 2H), |
| | | MS (ESP⁺) m/z 480.2 |
| 37 | 3-(benzo[d][1,3]dioxol-5-yl)-2- | ¹H NMR (400 MHz, CDCl₃) δ 8.85 (s, |
| | (pyridin-2-yl)-8-tosyl-1-oxa-8- | 1H), 7.80 (t, 1H), 7.65 (d, 2H), 7.55 (d, |
| | azaspiro[4.5]dec-2-en-4-one | 1H), 7.50 (t, 1H), 7.30 (d, 2H), 6.85 (d, |
| | | 1H), 6.75 (s, 1H), 6.74 (s, 1H), 5.95 (s, |
| | | 2H), 3.80 (br, 2H), 3.00 (d, 1H), 2.45 (s, |
| | | 3H), 2.20 (dt, 2H), 1.790 (d, 2H), |
| | | MS (ESP⁺) m/z 505.2 |
| 38 | 3-(benzo[d][1,3]dioxol-5-yl)-N- | ¹H NMR (400 MHz, CDCl₃) δ 8.80 (d, |
| | benzyl-4-oxo-2-(pyridin-2-yl)-1- | 1H), 7.80 (t, 1H), 7.60 (d, 1H), 7.50 (t, |
| | oxa-8-azaspiro[4.5]dec-2-ene-8- | 1H), 7.30 (d, 2H), 7.30 (m, 5H), 7.5-7.0 |
| | carboxamide | (m, 3H), 5.95 (s, 2H), 4.40 (s, 2H), 3.95 |
| | | (d, 2H), 3.20 (t, 1H), 2.20 (dt, 2H), 1.790 |
| | | (d, 2H), |
| | | MS (ESP⁺) m/z 484.2 |
| 39 | tert-butyl 2-(benzo[d][1,3]dioxol-5- | ¹H NMR (400 MHz, CDCl₃) δ 8.80 (d, |
| | yl)-4-oxo-3-(pyridin-2-yl)-1-oxa-8- | 1H), 8.12 (t, 1H), 7.75 (d, 1H), 7.55 (t, |
| | azaspiro[4.5]dec-2-ene-8- | 1H), 7.15 (d, 1H), 6.95 (s, 1H), 6.75 (d, |
| | carboxylate | 1H), 6.01 (s, 2H), 4.15 (br, 2H), 3.25 (br, |
| | | 1H), 2.00 (dt, 2H), 1.75 (d, 2H), 1.50 (s, |
| | | 9H) |
| | | MS (ESP⁺) m/z 451.02 |
| 40 | 2-(benzo[d][1,3]dioxol-5-yl)-3- | ¹H NMR (400 MHz, CDCl₃) δ 8.80 (d, |
| | (pyridin-2-yl)-8-tosyl-1-oxa-8- | 1H), 8.12 (t, 1H), 7.75 (d, 1H), 7.70 (d, |
| | azaspiro[4.5]dec-2-en-4-one | 2H), 7.55 (t, 1H), 7.40 (d, 2H), 6.95 (d, |
| | | 1H), 6.80 (s, 1H), 6.70 (d, 1H), 6.01 (s, |
| | | 2H), 3.80 (d, 2H), 2.95 (t, 1H), 2.50 (s, |
| | | 3H), 2.00 (dt, 2H), 1.95 (d, 2H) |
| | | MS (ESP⁺) m/z 504.97 |
| 41 | 2-(benzo[d][1,3]dioxol-5-yl)-8-(4- | ¹H NMR (400 MHz, CDCl₃) δ 8.70 (d, |
| | chlorophenylsulfonyl)-3-(pyridin-2- | 1H), 8.10 (t, 1H), 7.70 (d, 1H), 6.98 (d, |
| | yl)-1-oxa-8-azaspiro[4.5]dec-2-en- | 2H), 7.52 (t, 1H), 7.50 (d, 2H), 6.95 (d, |
| | 4-one | 1H), 6.80 (s, 1H), 6.70 (d, 1H), 5.95 (s, |
| | | 2H), 3.80 (d, 2H), 2.95 (t, 1H), 2.50 (s, |
| | | 3H), 2.00 (dt, 2H), 1.95 (d, 2H) |
| | | MS (ESP⁺) m/z 547.00 |
| 42 | 2-(benzo[d][1,3]dioxol-5-yl)-8-(3,4- | ¹H NMR (400 MHz, CDCl₃) δ 8.70 (d, |
| | dichlorophenylsulfonyl)-3-(pyridin- | 1H), 8.10 (t, 1H), 7.85 (s, 1H), 7.80 (d, |
| | 2-yl)-1-oxa-8-azaspiro[4.5]dec-2- | 1H), 7.60 (m, 3H), 6.98 (d, 2H), 6.95 (d, |
| | en-4-one | 1H), 6.80 (s, 1H), 6.70 (d, 1H), 5.95 (s, |
| | | 2H), 3.80 (d, 2H), 2.95 (t, 1H), 2.50 (s, |
| | | 3H), 2.00 (dt, 2H), 1.95 (d, 2H) |
| | | MS (ESP⁺) m/z 580.94/582.95 (M + Na) |
| 43 | 4-(2-(benzo[d][1,3]dioxol-5-yl)-4- | ¹H NMR (400 MHz, CDCl₃) δ 8.70 (d, |
| | oxo-3-(pyridin-2-yl)-1-oxa-8- | 1H), 8.15 (d, 2H), 8.10 (t, 1H), 7.85 (d, |
| | azaspiro[4.5]dec-2-en-8- | 2H), 7.75 (d, 1H), 7.60 (t, 1H), 6.95 (d, |
| | ylsulfonyl)benzoic acid | 1H), 6.80 (s, 1H), 6.70 (d, 1H), 5.95 (s, |
| | | 2H), 3.80 (d, 2H), 2.95 (t, 1H), 2.50 (s, |
| | | 3H), 2.00 (dt, 2H), 1.95 (d, 2H) |
| | | MS (ESP⁺) m/z 556.93 (M + Na) |
| 44 | 4-(2-(benzo[d][1,3]dioxol-5-yl)-4- | ¹H NMR (400 MHz, CDCl₃) δ 8.70 (d, |
| | oxo-3-(pyridin-2-yl)-1-oxa-8- | 1H), 8.10 (t, 1H), 7.90 (d, 2H), 7.80 (d, |
| | azaspiro[4.5]dec-2-en-8- | 2H), 7.75 (d, 1H), 7.60 (t, 1H), 6.95 (d, |
| | ylsulfonyl)benzonitrile | 1H), 6.80 (s, 1H), 6.70 (d, 1H), 5.95 (s, |
| | | 2H), 3.80 (d, 2H), 2.95 (t, 1H), 2.50 (s, |
| | | 3H), 2.00 (dt, 2H), 1.95 (d, 2H) |
| | | MS (ESP⁺) m/z 537.94 (M + Na) |
| 45 | 3-(benzo[d][1,3]dioxol-5-yl)-8-(4- | ¹H NMR (400 MHz, CDCl₃) δ 8.85 (s, |
| | chlorophenylsulfonyl)-2-(pyridin-2- | 1H), 7.85 (t, 1H), 7.65 (d, 2H), 7.50 (t, |
| | yl)-1-oxa-8-azaspiro[4.5]dec-2-en- | 2H), 7.40 (d, 2H), 6.70 (d, 1H), 6.75 (s, |
| | 4-one | 1H), 6.74 (s, 1H), 5.95 (s, 2H), 3.80 (br, |
| | | 2H), 3.00 (d, 1H), 2.45 (s, 3H), 2.20 (dt, |
| | | 2H), 1.790 (d, 2H), |
| | | MS (ESP⁺) m/z 547.0 |
| 46 | 3-(benzo[d][1,3]dioxol-5-yl)-8-(3,4- | ¹H NMR (400 MHz, CDCl₃) δ 8.85 (s, |
| | dichlorophenylsulfonyl)-2-(pyridin- | 1H), 7.85 (m, 2H), 7.50 (m, 4H), 6.70 (d, |
| | 2-yl)-1-oxa-8-azaspiro[4.5]dec-2- | 1H), 6.75 (s, 1H), 6.74 (s, 1H), 5.95 (s, |
| | en-4-one | 2H), 3.80 (br, 2H), 3.00 (d, 1H), 2.20 (dt, |
| | | 2H), 1.790 (d, 2H), |
| | | MS (ESP⁺) m/z 580.94/582.95 (M + Na) |
| 47 | 4-(3-(benzo[d][1,3]dioxol-5-yl)-4- | ¹H NMR (400 MHz, CDCl₃) δ 8.85 (s, |
| | oxo-2-(pyridin-2-yl)-1-oxa-8- | 1H), 8.10 (d, 2H), 7.82 (d, 2H), 7.70 (t, |
| | azaspiro[4.5]dec-2-en-8- | 1H), 7.55 (m, 2H), 6.75 (s, 1H), 6.74 (s, |
| | ylsulfonyl)benzoic acid | 1H), 5.95 (s, 2H), 3.80 (br, 2H), 3.00 (d, |
| | | 1H), 2.20 (dt, 2H), 1.790 (d, 2H), |
| | | MS (ESP⁺) m/z 556.93 |
| 48 | 4-(3-(benzo[d][1,3]dioxol-5-yl)-4- | ¹H NMR (400 MHz, CDCl₃) δ 8.85 (s, |
| | oxo-2-(pyridin-2-yl)-1-oxa-8- | 1H), 8.10 (d, 2H), 7.80 (d, 2H), 7.77 (t, |
| | azaspiro[4.5]dec-2-en-8- | 1H), 7.75 (d, 2H), 7.50 (m, 2H), 6.75 (s, |
| | ylsulfonyl)benzonitrile | 1H), 6.74 (s, 1H), 5.95 (s, 2H), 3.80 (br, |
| | | 2H), 3.00 (d, 1H), 2.20 (dt, 2H), 1.790 (d, 2H), |
| | | MS (ESP⁺) m/z 537.94 |
| 49 | 10-(quinoxalin-6-yl)-11-(3- | ¹H NMR, (400 MHz, CDCl₃) ä 8.90 (s, |
| | methylphenyl)-1,4,9-trioxa- | 2H), 8.59 (d, J = 1.6 Hz, 1H), 8.04 (d, J = |
| | dispiro[4.2.4.2]tetradec-10-en-12- | 9.2 Hz, 1H) 7.91 (d, J = 8.8 Hz, 1H), 7.29- |
| | one | 7.07 (m, 4H), 4.0 (s, 4H), 2.89-2.81 (m, |
| | | 2H), 2.67-2.41 (m, 2H), 2.33 (s, 3H), 2.43- |
| | | 2.33 (m, 2H), 2.29-2.23 (m, 2H); |
| | | MS ESI: 527 (M + 1). |
| 50 | 2-(quinoxalin-6-yl)-3-m-tolyl-1- | ¹H NMR, (400 MHz, CDCl₃) δ 8.90 (s, |
| | oxaspiro[4.5]dec-2-ene-4,8-dione | 2H), 8.59 (d, J = 1.6 Hz, 1H), 8.04 (d, J = |
| | | 9.2 Hz, 1H) 7.91 (d, J = 8.8 Hz, 1H), 7.29- |
| | | 7.07 (m, 4H), 2.89-2.81 (m, 2H), 2.67- |
| | | 2.41 (m, 2H), 2.33 (s, 3H), 2.43-2.33 (m, |
| | | 2H), 2.29-2.23 (m, 2H); |
| | | MS ESI: 385 (M + 1). |
| 51 | 10-(quinoxalin-6-yl)-11-(3- | ¹H NMR, (400 MHz, CDCl₃) δ 8.90 (s, |
| | chlorophenyl)-1,4,9-trioxa- | 2H), 8.59 (s, 1H), 8.17 (d, 1H) 7.87 (d, |
| | dispiro[4.2.4.2]tetradec-10-en-12- | 1H), 7.29-7.07 (m, 4H), 4.0 (s, 4H), 2.30- |
| | one | 1.81 (m, 8H), (m, 2H); |
| | | MS ESI: 448.9 (M + 1). |
| 52 | 3-(3-chlorophenyl)-2-(quinoxalin-6- | ¹H NMR, (400 MHz, CDCl₃) δ 9.0 (d, |
| | yl)-1-oxaspiro[4.5]dec-2-ene-4,8- | 2H), 8.65(s, 1H), 8.17 (d, 1H) 7.87 (d, |
| | dione | 1H), 7.29-7.07 (m, 4H), 2.85-2.25 (m, |
| | | 8H), (m, 2H); |
| | | MS ESI: 405.2 (M + 1). |
| 53 | 3-(3-chlorophenyl)-8-hydroxy-2- | ¹H NMR, (400 MHz, CDCl3) δ 8.94 (s, |
| | (quinoxalin-6-yl)-1- | 2H), 8.61 (d, J = 1.2 Hz, 1H), 8.06 (d, J = |
| | oxaspiro[4.5]dec-2-en-4-one | 8.8 Hz, 1H) 7.93 (d, J = 8.87 Hz, 1H), |
| | | 7.29-7.05 (m, 4H), 3.92-3.86 (sextet, J = |
| | | 5.1 Hz, 1H), 2.34 (s, 3H), 2.17-2.16 (m, |
| | | 2H), 2.14-1.82 (m, 6H). |
| | | MS ESI: 387 (M + 1). |
| 54 | 8-hydroxy-2-(quinoxalin-6-yl)-3-m- | ¹H NMR, (400 MHz, CDCl3) δ 8.92 (s, |
| | tolyl-1-oxaspiro[4.5]dec-2-en-4-one | 2H), 8.61 (s, 1H), 8.15 (d, 1H) 7.91 (d, |
| | | 1H), 7.25 (m, 2H), 7.17 (d, 1H), 7.05 (d, |
| | | 1H), 3.89 (m, 1H), 2.31 (s, 3H), 2.15 (m, |
| | | 2H), 2.0-1.8 (m, 6H); |
| | | MS ESI: 387.13 (M + 1). |
| 55 | ethyl 2-(4-oxo-2-(quinoxalin-6-yl)- | ¹H NMR, (400 MHz, CDCl₃) δ 8.96 (s, |
| | 3-m-tolyl-1-oxaspiro[4.5]dec-2-en- | 2H), 8.61 (d, J = 1.6 Hz, 1H), 8.07 (d, J = |
| | 8-ylidene)acetate | 8.8 Hz, 1H), 7.95 (d, J = 8.8 Hz, 1H), |
| | | 7.29-7.06 (m, 4H), 5.82 (s, 1H), 4.19 (q, J = |
| | | 7.2 Hz, 2H), 3.98 (d, J = 14.4 Hz, 1H), |
| | | 2.80-2.49 (m, 3 H), 2.34 (s, 3H), 2.17-2.05 |
| | | (m, 4H), 1.31 (t, J = 7.2 Hz, 3H). |
| | | MS ESI: 455 (M + 1). |
| 56 | 2-(4-oxo-2-(quinoxalin-6-yl)-3-m- | ¹H NMR, (400 MHz, CDCl₃) δ 8.93 (s, |
| | tolyl-1-oxaspiro[4.5]dec-2-en-8- | 2H), 8.66 (s, 1H), 8.05 (d, J = 8.8 Hz, 1H), |
| | ylidene)acetic acid | 7.93 (d, J = 8.8 Hz, 1H), 7.29-7.07 (m, |
| | | 4H), 5.87 (s, 1H), 3.98 (d, J = 14.0 Hz, |
| | | 1H), 2.34 (s, 3H), 2.66-2.02 (m, 7H). |
| | | MS ESI: 427 (M + 1). |
| 57 | 2-(4-oxo-2-(quinoxalin-6-yl)-3-m- | ¹H NMR, (400 MHz, CDCl₃) δ 8.91 (s, |
| | tolyl-1-oxaspiro[4.5]dec-2-en-8- | 2H), 8.58 (s, 1H), 8.02 (d, J = 8.8 Hz, 1H), |
| | yl)acetic acid | 7.90 (d, J = 8.8 Hz, 1H), 7.27-7.06 (m, |
| | | 4H), 5.87 (s, 1H), 3.98 (d, J = 14.0 Hz, |
| | | 1H), 2.52-1.89 (m, 11H) 2.34 (s, 3H),. |
| | | MS ESI: 429 (M + 1). |
| 58 | N-(2-morpholinoethyl)-2-(4-oxo-2- | ¹H NMR, (400 MHz, CDCl₃) δ 8.89 (s, |
| | (quinoxalin-6-yl)-3-m-tolyl-1- | 2H), 8.53 (s, 1H), 8.01 (d, J = 8.8 Hz, 1H), |
| | oxaspiro[4.5]dec-2-en-8- | 7.89 (d, J = 8.8 Hz, 1H), 7.74 (broad, 1H), |
| | yl)acetamide | 7.26-7.04 (m, 4H), 3.97-2.89 (m, 12H), |
| | | 2.32 (s, 3H), 2.60-1.88 (m, 11H). |
| | | MS ESI: 541 (M + 1). |
| 59 | N-(2-(dimethylamino)ethyl)-2-(4- | ¹H NMR, (400 MHz, CDCl₃) δ 8.88 (s, |
| | oxo-2-(quinoxalin-6-yl)-3-m-tolyl-1- | 2H), 8.52 (d, J = 1.2 Hz, 1H), 7.88 (d, J = |
| | oxaspiro[4.5]dec-2-en-8- | 8.8 Hz, 1H), 7.83 (s, 1H), 7.24 (d, J = 7.6 |
| | yl)acetamide | Hz, 1H), 7.14 (s, 2H), 7.04 (d, J = 7.6 Hz, |
| | | 1H), 3.67 (d, J = 3.2 Hz, 2H), 3.23 (s, 2H), |
| | | 2.86 (s, 6H), 2.32 (s, 3H), 2.32-1.88 (m, |
| | | 10H). |
| | | MS ESI: 499 (M + 1). |
| 60 | ethyl 2-(3-(3-chlorophenyl)-4-oxo- | ¹H NMR, (400 MHz, CDCl₃) δ 9.00 (s, |
| | 2-(quinoxalin-6-yl)-1- | 2H), 8.61 (d, 1H), 8.13 (d, 1H), 7.93 (dd, |
| | oxaspiro[4.5]dec-2-en-8- | 1H), 7.35 (m, 3H), 7.20 (m, 1H), 5.71 (s, |
| | ylidene)acetate | 1H), 4.20 (q, 2H), 3.98 (m, 1H), 2.80-2.49 |
| | | (m, 3 H), 2.10 (m, 4H), 1.31 (t, 3H). |
| | | MS ESI: 474.8 (M + 1). |
| 61 | 2-(3-(3-chlorophenyl)-4-oxo-2- | ¹H NMR, (400 MHz, CDCl₃) δ 9.00 (s, |
| | (quinoxalin-6-yl)-1- | 2H), 8.61 (d, 1H), 8.13 (d, 1H), 7.93 (dd, |
| | oxaspiro[4.5]dec-2-en-8-yl)acetic | 1H), 7.35 (m, 3H), 7.20 (m, 1H), 2.5 (d, |
| | acid | 2H), 2.10-1.9 (m, 9H), |
| | | MS ESI: 449.0 (M + 1). |
| 62 | N-methyl-2-(4-oxo-2-(quinoxalin-6- | ¹H NMR, (400 MHz, CDCl₃) δ 8.91 (s, |
| | yl)-3-m-tolyl-1-oxaspiro[4.5]dec-2- | 2H), 8.58 (d, 1H), 8.05 (d, 1H), 7.92 (dd, |
| | en-8-yl)acetamide | 1H), 7.27-7.06 (m, 4H), 2.71 (d, 3H), 2.34 |
| | | (s, 3H),. 2.30 (br, 2H), 2.1-1.8 (m, 8H). |
| | | MS ESI: 442.1 (M + 1). |
| 63 | N-(2-methoxyethyl)-2-(4-oxo-2- | ¹H NMR, (400 MHz, CDCl₃) δ 8.88 (d, |
| | (quinoxalin-6-yl)-3-m-tolyl-1- | 2H), 8.37 (s, 1H), 8.01 (d, 1H), 7.90 (d, |
| | oxaspiro[4.5]dec-2-en-8- | 1H), 7.30-6.95 (m, 4H), 3.45-3.20 (m, |
| | yl)acetamide | 7H), 2.30 (s, 3H),. 2.40-1.75 (m, 11H). |
| | | MS ESI: 486.1 (M + 1). |
| 64 | 2-(3-(3-chlorophenyl)-4-oxo-2- | ¹H NMR, (400 MHz, CDCl₃) δ 8.89 (s, |
| | (quinoxalin-6-yl)-1- | 2H), 8.50 (s, 1H), 8.08 (d, 1H), 7.85 (d, |
| | oxaspiro[4.5]dec-2-en-8-yl)-N-(2- | 1H), 7.74 (broad, 1H), 7.26-7.04 (m, 4H), |
| | morpholinoethyl)acetamide | 4.0 (s, 4H), 3.25 (s, 2H), 2.80 (m, 2H), |
| | | 2.30-1.70 (m, 11H). |
| | | MS ESI: 561.1 (M + 1). |
| 65 | N-(2-([1,2,4]triazolo[1,5-a]pyridin- | ¹H NMR (CDCl₃, 300 MHz): δ 9.17 (s, |
| | 6-yl)-4-oxo-3-m-tolyl-1- | 1H), 8.54 (s, 1H), 7.75 (s, 2H), 7.35-7.09 |
| | oxaspiro[4.5]dec-2-en-8- | (m, 4H), 3.87(m, 1H), 2.36 (s, 3H), 2.04 |
| | yl)acetamide | (s, 3H), 2.10-1.80 (m, 8H) |
| | | MS ESI: 417.22 (M + 1). |
| 66 | 1-(2-([1,2,4]triazolo[1,5-a]pyridin- | ¹H NMR (CDCl₃₎ 300 MHz): δ 9.17 (s, |
| | 6-yl)-4-oxo-3-m-tolyl-1- | 1H), 8.88 (s, 1H), 7.78 (s, 2H), 7.35-7.09 |
| | oxaspiro[4.5]dec-2-en-8-yl)urea | (m, 4H), 3.67 (m, 1H), 2.36 (s, 3H), 2.10- |
| | | 1.80 (m, 8H) |
| | | MS ESI: 418.24 (M + 1). |
| 67 | 8-aminosulfonylamino-2- | ¹H NMR (CDCl₃, 300 MHz): δ 9.17 (s, |
| | ([1,2,4]triazolo[1,5-a]pyridin-6-yl)- | 1H), 8.52 (s, 1H), 7.75 (s, 2H), 7.35-7.09 |
| | 3-m-tolyl-1-oxaspiro[4.5]dec-2-en- | (m, 4H), 3.70 (m, 1H), 2.36 (s, 3H), 2.10- |
| | 4-one | 1.80 (m, 8H) |
| | | MS ESI: 454.27 (M + 1). |
| 68 | 2-([1,2,4]triazolo[1,5-a]pyridin-6- | ¹H NMR (CDCl₃, 300 MHz): δ 9.23 (s, |
| | yl)-8-hydroxy-3-(6-methylpyridin- | 1H), 8.37 (s, 1H), 8.14 (t, 1H), 7.67-7.51 |
| | 2-yl)-1-oxaspiro[4.5]dec-2-en-4-one | (m, 4H), 3.60 (m, 1H), 2.45 (s, 3H), 1.94- |
| | | 1.59 (m, 8H) |
| | | MS ESI: 377.30 (M + 1). |
| 69 | 2-(4-oxo-2-(quinoxalin-6-yl)-3-m- | ¹H NMR, (400 MHz, CDCl₃) δ 8.93 (s, |
| | tolyl-1-oxaspiro[4.5]dec-2-en-8- | 2H), 8.56 (s, 1H), 8.05 (d, J = 8.4 Hz, 1H), |
| | yl)acetamide | 7.93 (d, J = 8.4 Hz, 1H), 7.29-7.04 (m, |
| | | 4H), 6.96 (broad, 1H), 6.25 (broad, 1H), |
| | | 2.41 (d, J = 7.6 Hz, 2H), 2.34 (s, 3H), |
| | | 2.26-1.87 (m, 9H). |
| | | MS ESI: 428 (M + 1). |
| 70 | 2-(2-([1,2,4]triazolo[,5-a]pyridin- | ¹H NMR (CDCl₃, 300 MHz): δ 9.17 (s, |
| | 6-yl)-4-oxo-3-m-tolyl-1- | 1H), 8.50 (s, 1H), 7.79 (d, 1H), 7.71 (d, |
| | oxaspiro[4.5]dec-2-en-8-ylamino)- | 1H), 7.34-7.04 (m, 4H), 6.21 (d, 1H), 4.62 |
| | 2-oxoethyl acetate | (s, 1H), 4.06 (m, 1H), 2.37 (s, 3H), 1. 2.24 |
| | | (s, 3H), 2.11-1.41 (m, 8H) |
| | | MS ESI: 475.24 (M + 1). |
| 71 | methyl 3-(2-([1,2,4]triazolo[1,5- | ¹H NMR (CDCl₃, 300 MHz): δ 9.21 (s, |
| | a]pyridin-6-yl)-4-oxo-3-m-tolyl-1- | 1H), 8.57 (s, 1H), 7.88 (d, 1H), 7.81 (d, |
| | oxaspiro[4.5]dec-2-en-8-ylamino)- | 1H), 7.58 (d, 1H), 7.34-7.04 (m, 4H), 4.62 |
| | 3-oxopropanoate | (s, 1H), 4.13 (m, 1H), 3.85 (s, 3H), 3.49 (s, |
| | | 3H), 2.45 (s, 3H), 2.24 (s, 3H), 2.28-1.82 |
| | | (m, 8H) |
| | | MS ESI: 475.24 (M + 1). |
| 72 | dimethyl 3,3′-(2-([1,2,4]triazolo[1,5- | ¹H NMR (CDCl₃, 300 MHz): δ 9.54 (d, |
| | a]pyridin-6-yl)-4-oxo-3-m-tolyl-1- | 1H), 9.27 (s, 1H), 8.68 (s, 1H), 8.04 (d, |
| | oxaspiro[4.5]dec-2-en-8- | 1H), 7.95 (d, 1H), 7.50-7.19 (m, 4H), 4.62 |
| | ylazanediyl)bis(3-oxopropanoate) | (s, 1H), 4.22 (m, 1H), 3.95 (s, 3H), 3.90 (s, |
| | | 3H), 2.52 (s, 3H), 2.37 (s, 3H), 2.28-1.82 |
| | | (m, 8H) |
| | | MS ESI: 573.30 (M + 1). |
| 73 | 3-(2-([1,2,4]triazolo[1,5-a]pyridin- | ¹H NMR (CDCl₃, 300 MHz): δ 9.21 (s, |
| | 6-yl)-4-oxo-3-m-tolyl-1- | 1H), 8.57 (s, 1H), 7.87 (d, 1H), 7.81 (d, |
| | oxaspiro[4.5]dec-2-en-8-ylamino)- | 1H), 7.34-7.04 (m, 4H), 6.95 (t, 1H), 4.26 |
| | 3-oxopropanoic acid | (s, 2H), 4.14 (m, 1H), 2.43 (s, 3H), 2.28- |
| | | 1.80 (m, 8H) |
| | | MS ESI: 433.21 (M + 1). |
| 74 | N-hydroxy-2-(4-oxo-2-(quinoxalin- | ¹H NMR, (400 MHz, CDCl₃) δ 8.90 (s, |
| | 6-yl)-3-m-tolyl-1-oxaspiro[4.5]dec- | 2H), 8.55 (s, 1H), 8.03 (d, J = 8.8 Hz, 1H), |
| | 2-en-8-yl)acetamide | 7.90 (d, J = 8.8 Hz, 1H), 7.25-7.05 (m, |
| | | 4H), 2.33 (s, 3H), 2.07-1.80 (m, 9H). |
| | | MS ESI: 444 (M + 1). |
| 75 | 2-(2-([1,2,4]triazolo[1,5-a]pyridin- | ¹H NMR, (400 MHz, CDCl₃) δ 9.08 (s, |
| | 6-yl)-4-oxo-3-m-tolyl-1- | 1H), 8.86 (s, 1H), 8.63 (s, 1H), 8.40 (s, |
| | oxaspiro[4.5]dec-2-en-8-ylidene)-N- | 1H), 8.32 (d, J = 6.8 Hz, 1H), 7.78 (s, 1H), |
| | (pyridin-3-ylmethyl)acetamide | 7.64 (q, J = 8.0 Hz, 2H), 7.31-7.06 (m, |
| | | 4H), 6.86 (t, J = 13.2 Hz, 1H), 4.61 (d, J = |
| | | 4.4 Hz, 2H), 4.01 (d, J = 13.6 Hz, 1H), |
| | | 2.70-2.00 (m, 7H), 2.34 (s, 3H). |
| | | MS ESI: 506 (M + 1) |
| 76 | 11-(3-chloro-4-fluoro-phenyl)-10- | ¹H NMR, (400 MHz, CDCl₃) δ 9.12 (s, |
| | [1,2,4]triazolo[1,5-a]pyridin-6-yl- | 1H), 8.56 (s, 1H), 7.94 (d, J = 9.2 Hz, 1H), |
| | 1,4,9-trioxa-dispiro[4.2.4.2]tetradec- | 7.73 (d, J = 9.2 Hz, 1H), 7.40 (d, J = 9.2 |
| | 10-en-12-one | Hz, 1H), 7.22-7.14 (m, 2H), 4.04 (seven, J = |
| | | 4.7 Hz, 4H), 2.28-1.88 (m, 8H). |
| | | MS ESI: 456 (M + 1). |
| 77 | 3-(3-chloro-4-fluoro-phenyl)-2- | ¹H NMR, (400 MHz, CDCl₃) δ 9.16 (s, |
| | [1,2,4]triazolo[1,5-a]pyridin-6-yl-1- | 1H), 8.52 (s, 1H), 7.87 (d, J = 6.8 Hz, 1H), |
| | oxa-spiro[4.5]dec-2-ene-4,8-dione | 7.68 (d, J = 6.8 Hz, 1H), 7.40 (d, J = 6.8 |
| | | Hz, 1H), 7.22-7.09 (m, 2H), 4.04 (seven, J = |
| | | 4.7 Hz, 4H), 2.84-2.23 (m, 8H). |
| | | MS ESI: 412 (M + 1). |
| 78 | ethyl 2-(2-([1,2,4]triazolo[1,5- | ¹H NMR, (400 MHz, CDCl₃) δ 9.17 (s, |
| | a]pyridin-6-yl)-3-(3-chloro-4- | 1H), 8.58 (s, 1H), 7.98 (d, J = 9.2 Hz, 1H), |
| | fluorophenyl)-4-oxo-1- | 7.75 (d, J = 9.2 Hz, 1H), 7.40 (d, J = 9.2 |
| | oxaspiro[4.5]dec-2-en-8- | Hz, 1H), 7.23-7.14 (m, 2H), 5.82 (s, 1H), |
| | ylidene)acetate | 4.21 (q, J = 7.0 Hz, 2H), 3.95 (d, J = |
| | | 14.0 Hz, 1H), 2.72-2.51 (m, 3H), 2.15-2.04 |
| | | (m, 4H), 1.31 (t, J = 7.0 Hz, 3H). |
| | | MS ESI: 481 (M + 1). |
| 79 | 5-([1,2,4]triazolo[1,5-a]pyridin-6- | ¹H NMR (300 MHz, CD₃OD) δ: 9.17 (s, |
| | yl)-4-(3-chlorophenyl)-2,2- | 1H), 8.52 (s, 1H), 7.73 (m, 2H), 7.40 (m, |
| | dimethylfuran-3(2H)-one | 3H), 7.24 (m, 1H), 1.59 (s, 6H). |
| | | MS (ESP⁺) m/z 340.11. |
| 80 | 5-([1,2,4]triazolo[1,5-a]pyridin-6- | ¹H NMR (300 MHz, CD₃OD) δ 9.19 (s, |
| | yl)-4-(3-chloro-4-fluorophenyl)-2,2- | 1H), 8.49 (s, 1H), 7.75 (m, 3H), 7.29 (m, |
| | dimethylfuran-3(2H)-one | 2H), 1.59 (s, 6H). |
| | | MS (ESP⁺) m/z 358.09. |

### Example 81

### Cell-Free Assay for Evaluating Inhibition of Autophosphorylation of TGFβ Type I Receptor

The serine-threonine kinase activity of TGFβ type I receptor was measured as the autophosphorylation activity of the cytoplasmic domain of the receptor containing an N-terminal poly histidine, TEV cleavage site-tag, e.g., His-TGFβRI. The His-tagged receptor cytoplasmic kinase domains were purified from infected insect cell cultures using the Gibco-BRL FastBac HTb baculovirus expression system.

To a 96-well Nickel FlashPlate (NEN Life Science, Perkin Elmer) was added 20 μL of 1.25 μCi ³³P-ATP/25 μM ATP in assay buffer (50 mM Hepes, 60 mM NaCl, 1 mM MgCl₂, 2 mM DTT, 5 mM MnCl₂, 2% glycerol, and 0.015% Brij® 35). 10 μL of each test compound of Formula (I) prepared in 5% DMSO solution were added to the FlashPlate. The assay was then initiated with the addition of 20 μL of assay buffer containing 12.5 pmol of His-TGFβRI to each well. Plates were incubated for 30 minutes at room temperature and the reactions were then terminated by a single rinse with TBS. Radiation from each well of the plates was read on a TopCount (Packard). Total binding (no inhibition) was defined as counts measured in the presence of DMSO solution containing no test compound and non-specific binding was defined as counts measured in the presence of EDTA or no-kinase control.

Alternatively, the reaction performed using the above reagents and incubation conditions but in a microcentrifuge tube was analyzed by separation on a 4-20% SDS-PAGE gel and the incorporation of radiolabel into the 40 kDa His-TGFβRI SDS-PAGE band was quantitated on a Storm Phosphoimager (Molecular Dynamics).

Compounds of Formula (I) typically exhibited IC₅₀ values of less than 10 μM; some exhibited IC₅₀ values of less than 1 μM; and some even exhibited IC₅₀ values of less than 50 nM.

### Example 82

### Cell-Free Assay for Evaluating Inhibition of Activin Type I Receptor Kinase Activity

Inhibition of the Activin type I receptor (Alk 4) kinase autophosphorylation activity by test compounds of Formula (I) can be determined in a similar manner to that described above in Example 34 except that a similarly His-tagged forM Alk 4 (His-Alk 4) is used in place of the His-TGFβRI.

### Example 83

### TGFβ Type I Receptor Ligand Displacement FlashPlate Assay

50 nM tritiated 4-(3-pyridin-2-yl-1H-pyrazol-4-yl)-quinoline (custom-ordered from PerkinElmer Life Science, Inc., Boston, Mass.) in assay buffer (50 mM Hepes, 60 mM NaCl₂, 1 mM MgCl₂, 5 mM MnCl₂, 2 mM 1,4-dithiothreitol (DTT), 2% Brij® 35; pH 7.5) was premixed with a test compound of Formula (I) in 1% DMSO solution in a v-bottom plate. Control wells containing either DMSO without any test compound or control compound in DMSO were used. To initiate the assay, His-TGFβ Type I receptor in the same assay buffer (Hepes, NaCl₂, MgCl₂, MnCl₂, DTT, and 30% Brij® added fresh) was added to a nickel coated FlashPlate (PE, NEN catalog number: SMP107), while the control wells contained only buffer (i.e., no His-TGFβ Type I receptor). The premixed solution of tritiated 4-(3-pyridin-2-yl-1H-pyrazol-4-yl)-quinoline and test compound of Formula (I) was then added to the wells. The wells were aspirated after an hour at room temperature and radioactivity in wells (emitted from the tritiated compound) was measured using TopCount (PerkinElmer Lifesciences, Inc., Boston Mass.).

Compounds of Formula (I) typically exhibited K, values of less than 10 μM; some exhibited IC, values of less than 1 μM; and some even exhibited K, values of less than 50 nM.

### Example 84

### Assay for Evaluating Cellular Inhibition of TGFβ Signaling and Cytotoxicity

Biological activity of the compounds of Formula (I) was determined by measuring their ability to inhibit TGFβ-induced PAI-Luciferase reporter activity in HepG2 cells.

HepG2 cells were stably transfected with the PAI-luciferase reporter grown in DMEM medium containing 10% FBS, penicillin (100 U/mL), streptomycin (100 μg/mL), L-glutamine (2 mM), sodium pyruvate (1 mM), and non-essential amino acids (1×). The transfected cells were then plated at a concentration of 2.5×10⁴ cells/well in 96 well plates and starved for 3-6 hours in media with 0.5% FBS at 37° C. in a 5% CO₂ incubator. The cells were then stimulated with 2.5 ng/mL TGFβ ligand in the starvation media containing 1% DMSO either in the presence or absence of a test compound of Formula (I) and incubated as described above for 24 hours. The media was washed out the following day and the luciferase reporter activity was detected using the LucLite Luciferase Reporter Gene Assay kit (Packard, cat. no. 6016911) as recommended. The plates were read on a Wallac Microbeta plate reader, the reading of which was used to determine the IC₅₀ values of compounds of Formula (I) for inhibiting TGFβ-induced PAI-Luciferase reporter activity in HepG2 cells. Compounds of Formula (I) typically exhibited IC₅₀ values of less 10 μM.

Cytotoxicity was determined using the same cell culture conditions as described above. Specifically, cell viability was determined after overnight incubation with the CytoLite cell viability kit (Packard, Cat. No. 6016901). Compounds of Formula (I) typically exhibited LD₂₅ values greater than 10 μM.

### Example 85

### Assay for Evaluating Inhibition of TGFβ Type I Receptor Kinase Activity in Cells

The cellular inhibition of activin signaling activity by the test compounds of Formula (I) is determined in a similar manner as described above in Example 37 except that 100 ng/mL of activin is added to serum starved cells in place of the 2.5 ng/mL TGFβ.

### Example 86

### Assay for TGFβ-Induced Collagen Expression

### Step 1: Preparation of Immortalized Collagen Promotor-Green Fluorescent Protein Cells

Fibroblasts are derived from the skin of adult transgenic mice expressing Green Fluorescent Protein (GFP) under the control of the collagen 1Al promoter (see Krempen, K. et al., Gene Exp. 8: 151-163 (1999)). Cells are immortalized with a temperature sensitive large T antigen that is in an active stage at 33° C. Cells are expanded at 33° C. and then transferred to 37° C. at which temperature the large T antigen becomes inactive (see Xu, S. et al., Exp. Cell Res., 220: 407-414 (1995)). Over the course of about 4 days and one split, the cells cease proliferating. Cells are then frozen in aliquots sufficient for a single 96 well plate.

### Step 2: Assay of TGFβ-induced Collagen-GFP Expression

Cells are thawed, plated in complete DMEM (contains non-essential amino acids, 1 mM sodium pyruvate and 2 mM L-glutamine) with 10% fetal calf serum, and then incubated for overnight at 37° C., 5% CO₂. The cells are trypsinized in the following day and transferred into 96 well format with 30,000 cells per well in 50 μL complete DMEM containing 2% fetal calf serum, but without phenol red. The cells are incubated at 37° C. for 3 to 4 hours to allow them to adhere to the plate. Solutions containing a test compound of Formula (I) are then added to wells with no TGFβ (in triplicates), as well as wells with 1 ng/mL TGFβ (in triplicates). DMSO is also added to all of the wells at a final concentration of 0.1%. GFP fluorescence emission at 530 nm following excitation at 485 nm is measured at 48 hours after the addition of solutions containing a test compound on a CytoFluor microplate reader (PerSeptive Biosystems). The data are then expressed as the ratio of TGFβ-induced to non-induced for each test sample.

### Example 87

### Fluorescence Polarization Assay for Evaluating Inhibition of TGFβ Receptor

Competitive displacement using a fluorescence polarization assay utilized an Oregon green-labeled ALK4/5 inhibitor, which was shown to bind with high affinity to ALK5 (Kd, 0.34+0.01 nmol/L) and ALK4 (Kd, 0.53+0.03 nmol/L), using fluorescence polarization saturation curve analysis. Varying concentrations of compounds of Formula (I) and 25 nmol/L of the Oregon Green-labeled ALK4/5 inhibitor were incubated (1 hour, room temperature, in the dark) with 4.5 nmol/L of hALK4-K or hALK5-K, 30 mmol/L Hepes pH 7.5, 20 mmol/L NaCl, 1 mmol/L MgCl₂, 100 mmol/L KCl, 0.01% BSA, 0.01% Tween-20 at a final concentration of 1% DMSO in black 96-well Microfluor 2 plates (Cat. No. 7205, ThermoLab Systems). The signal was detected at excitation/emission settings of 490/530 nanometers using an Analyst HT (LJL BioSystems, Sunnyvale, Calif.). The IC₅₀ values for the tested compounds of Formula (I) were determined by nonlinear regression and their Ki values were calculated from the Cheng-Prusoff equation.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-55. (canceled)

56. A compound of Formula (I), 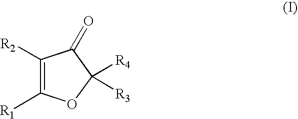
an N-oxide derivative, or a pharmaceutically acceptable salt thereof, wherein:
R¹ is an optionally substituted aryl or heteroaryl;
R² is an optionally substituted aryl or heteroaryl; and
Each of R³ and R⁴ is independently an optionally substituted aliphatic, an optionally substituted aryl, or an optionally substituted heteroaryl; or R³ and R⁴, together with the atom to which they are attached, form an optionally substituted 5- to 8-membered cycloaliphatic or an optionally substituted 5- to 8-membered heterocycloaliphatic ring.

57. The compound of claim 56, wherein R¹ is an optionally substituted aryl, or optionally substituted heteroaryl; substituted with 1 to 3 substituents each independently selected from the group consisting of alkyl, alkenyl, alkynyl, alkoxy, acyl, halo, hydroxy, amino, nitro, cyano, guanadino, amidino, carboxy, amido, alkoxycarbonyl, alkylcarbonyloxy, urea, thiourea, carbamoyl, cycloalkyl, cycloalkyloxy, cycloalkylcarbonyl, heterocycloalkyl, heterocycloalkyloxy, heterocycloalkylcarbonyl, aryl, aryloxy, arylsulfanyl, aroyl, heteroaryl, heteroaryloxy, and heteroaroyl.

58. The compound of claim 57, wherein R¹ is an optionally substituted heteroaryl.

59. The compound of claim 58, wherein R¹ is optionally substituted pyridyl or pyrimidinyl.

60. The compound of claim 59, wherein R¹ is benzo[1,3]dioxolyl, benzo[b]thiophenyl, benzooxadiazolyl, benzothiadiazolyl, benzoimidazolyl, benzooxazolyl, benzothiazolyl, 2-oxo-benzooxazolyl, 2,3-dihydrobenzo[1,4]dioxyl, 2,3-dihydrobenzofuryl, 2,3-dihydrobenzo[b]thiophenyl, 3,4-dihydrobenzo[1,4]oxazinyl, 3-oxo-benzo[1,4]oxazinyl, 1,1-dioxo-2,3 dihydrobenzo[b]thiophenyl, [1,2,4]triazolo[1,5 a]pyridyl, [1,2,4]triazolo[4,3 a]pyridyl, quinolinyl, quinoxalinyl, quinazolinyl, isoquinolinyl, or cinnolinyl; wherein each R¹ may be optionally substituted with 1 to 3 substituents each independently selected from the group consisting of: alkyl, alkenyl, alkynyl, alkoxy, acyl, halo, hydroxy, amino, nitro, oxo, thioxo, cyano, guanadino, amidino, carboxy, sulfo, mercapto, alkylsulfanyl, alkylsulfinyl, alkylsulfonyl, amido, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, cycloalkylcarbonyl, heterocycloalkyl, heterocycloalkyloxy, heterocycloalkylsulfanyl, heterocycloalkylcarbonyl, aryl, aryloxy, arylsulfanyl, aroyl, heteroaryl, heteroaryloxy, heteroarylsulfanyl, and heteroaroyl.

61. The compound of claim 60, wherein R¹ is benzo[1,3]dioxolyl, [1,2,4]triazolo[1,5 a]pyridin-6-yl, or quinoxalin-6-yl, or optionally substituted benzo[1,3]dioxolyl, optionally substituted [1,2,4]triazolo[1,5-a]pyridin-6-yl, or optionally substituted quinoxalin-6-yl.

62. The compound of claim 61, wherein R² is optionally substituted phenyl

63. The compound of claim 62, wherein R² is phenyl, optionally substituted with 1 to 3 substituents each independently selected from the group consisting of alkyl, alkenyl, alkynyl, alkoxy, acyl, halo, hydroxy, amino, nitro, oxo, thioxo, cyano, guanadino, amidino, carboxy, sulfo, mercapto, alkylsulfanyl, alkylsulfinyl, alkylsulfonyl, amido, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, cycloalkylcarbonyl, heterocycloalkyl, heterocycloalkyloxy, heterocycloalkylsulfanyl, heterocycloalkylcarbonyl, aryl, aryloxy, arylsulfanyl, aroyl, heteroaryl, heteroaryloxy, heteroarylsulfanyl, and heteroaroyl.

64. The compound of claim 63, wherein R² is o-, in-, or p-methylphenyl, chlorophenyl or fluorophenyl.

65. The compound of claim 64, wherein R² is an optionally substituted heteroaryl.

66. The compound of claim 65, wherein R² is optionally substituted pyridyl or optionally substituted pyrimidinyl optionally substituted with 1 to 3 substituents each independently selected from the group consisting of alkyl, alkenyl, alkynyl, alkoxy, acyl, halo, hydroxy, amino, nitro, oxo, thioxo, cyano, guanadino, amidino, carboxy, sulfo, mercapto, alkylsulfanyl, alkylsulfinyl, alkylsulfonyl, amido, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, cycloalkylcarbonyl, heterocycloalkyl, heterocycloalkyloxy, heterocycloalkylsulfanyl, heterocycloalkylcarbonyl, aryl, aryloxy, arylsulfanyl, aroyl, heteroaryl, heteroaryloxy, heteroarylsulfanyl, and heteroaroyl.

67. The compound of claim 66, wherein R² is selected from the group consisting of benzo[1,3]dioxolyl, benzo[b]thiophenyl, benzooxadiazolyl, benzothiadiazolyl, benzoimidazolyl, benzooxazolyl, benzothiazolyl, 2-oxo-benzooxazolyl, 2,3-dihydrobenzo[1,4]dioxyl, 2,3-dihydrobenzofuryl, 2,3-dihydrobenzo[b]thiophenyl, 3,4-dihydrobenzo[1,4]oxazinyl, 3-oxo-benzo[1,4]oxazinyl, 1,1-dioxo-2,3-dihydrobenzo[b]thiophenyl, [1,2,4]triazolo[1,5a]pyridyl, [1,2,4]triazolo[4,3a]pyridyl, quinolinyl, quinoxalinyl, quinazolinyl, isoquinolinyl, and cinnolinyl; and R² is optionally substituted.

68. The compound of claim 67, wherein R² is optionally substituted benzo[1,3]dioxolyl.

69. The compound of claim 56, wherein R³ and R⁴ together with the atom to which they are attached form an optionally substituted 5- to 8-membered cycloaliphatic ring compound of Formula (Ia), 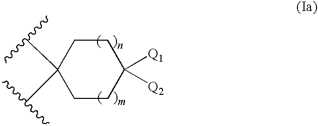
wherein:
Each of m and n is independently 0, 1, 2, 3 or 4, provided that the sum of m and n is 1, 2, 3, 4 or 5; and
Each of Qᵢ and Q₂ is independently H, alkyl, alkenyl, alkynyl, alkoxy, acyl, halo, hydroxy, amino, azido, nitro, cyano, guanadino, amidino, carboxy, sulfa, mercapto, alkylsulfanyl, alkylsulfinyl, alkylsulfonyl, arylsulfonyl, amido, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, cycloalkylcarbonyl, heterocycloalkyl, heterocycloalkyloxy, heterocycloalkylsulfanyl, heterocycloalkylcarbonyl, aryl, aryloxy, arylsulfanyl, aroyl, heteroaryl, heteroaryloxy, heteroarylsulfanyl, or heteroaroyl; or
Qᵢ and Q₂, together with the atom to which they are attached, form oxo, optionally substituted imino, or optionally substituted alkene; or
Qᵢ and Q₂, together with the atom to which they are attached, form an optionally substituted 5- to 7-membered cycloaliphatic or an optionally substituted 5- to 7-membered heterocycloaliphatic ring.

70. The compound of claim 69, wherein Qᵢ is H; and Q₂ is hydroxy, alkoxy, alkylcarbonyloxy, or carbamoyl, alkoxycarbonyl substituted aliphatic, carboxy substituted aliphatic, or amido substituted aliphatic,

71. The compound of claim 69, wherein Q₁ is H; and Q₂ is amino, azido, alkylsulfonylamino, arylsulfonylamino, alkylamido, arylamido, heteroarylamido, urea or aminosulfonylamino.

72. The compound of claim 69, wherein Qᵢ and Q₂, together with the atom to which they are attached, form oxo or optionally substituted imino or a 5- to 7-membered cycloaliphatic or a 5- to 7-membered heterocycloaliphatic ring.

73. The compound of claim 56, wherein R³ and R⁴, together with the atom to which they are attached, form an optionally substituted 5- to 8-membered heterocycloaliphatic ring of Formula (Ib), 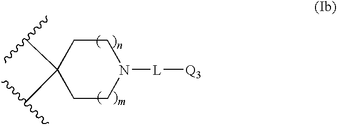
wherein:
Each of m and n is independently 0, 1, 2, 3 or 4, provided that the sum of m and n is 1, 2, 3, 4 or 5;
L is a bond, C(O) or S(O)ₚ;
p is 0, 1 or 2; and
Q₃ is H, optionally substituted aliphatic, optionally substituted aryl, optionally substituted cycloaliphatic, optionally substituted heterocycloaliphatic, amino, amido optionally substituted alkoxy, or optionally substituted aryloxy.

74. The compound of claim 73, wherein, each of m and n is independently 1 and L-Q₃ is H, alkoxycarbonyl, or amido.

75. The compound of claim 74, wherein, each of m and n is independently 1 and L-Q₃ is acyl, aroyl, alkylsulfonyl, or arylsulfonyl.

76. The compound of claim 56, wherein the compound is
tert-butyl 3-(benzo[d][1,3]dioxol-5-yl)-4-oxo-2-(pyridin-4-yl)-1-oxa-8-azaspiro[4.5]dec-2-ene-8-carboxylate;
benzyl 3-(benzo[d][1,3]dioxol-5-yl)-4-oxo-2-(pyridin-4-yl)-1-oxa-8-azaspiro[4.5]dec-2-ene-8-carboxylate;
3-(benzo[d][1,3]dioxol-5-yl)-2-(pyridin-4-yl)-1-oxa-8-azaspiro[4.5]dec-2-en-4-one;
4-(3-(benzo[d][1,3]dioxol-5-yl)-4-oxo-2-(pyridin-4-yl)-1-oxa-8-azaspiro[4.5]dec-2-enecarbonyl)benzonitrile;
tert-butyl 2-(benzo[d][1,3]dioxol-5-yl)-4-oxo-3-(pyridin-3-yl)-1-oxa-8-azaspiro[4.5]dec-2-ene-8-carboxylate;
tert-butyl 2-(benzo[d][1,3]dioxol-5-yl)-4-oxo-3-(pyridin-4-yl)-1-oxa-8-azaspiro[4.5]dec-2-ene-8-carboxylate;
2-(benzo[d][1,3]dioxol-5-yl)-3-(pyridin-3-yl)-1-oxa-8-azaspiro[4.5]dec-2-en-4-one;
benzyl 2-(benzo[d][1,3]dioxol-5-yl)-4-oxo-3-(pyridin-4-yl)-1-oxa-8-azaspiro[4.5]dec-2-ene-8-carboxylate;
benzyl 2-(benzo[d][1,3]dioxol-5-yl)-4-oxo-3-(pyridin-3-yl)-1-oxa-8-azaspiro[4.5]dec-2-ene-8-carboxylate;
2-(benzo[d][1,3]dioxol-5-yl)-8-(4-hydroxybenzoyl)-3-(pyridin-3-yl)-1-oxa-8-azaspiro[4.5]dec-2-en-4-one;
2-(benzo[d][1,3]dioxol-5-yl)-3-(pyridin-3-yl)-8-tosyl-1-oxa-8-azaspiro[4.5]dec-2-en-4-one;
tert-butyl 3-(benzo[d][1,3]dioxol-5-yl)-4-oxo-2-(pyridin-2-yl)-1-oxa-8-azaspiro[4.5]dec-2-ene-8-carboxylate;
3-(benzo[d][1,3]dioxol-5-yl)-2-(pyridin-2-yl)-1-oxa-8-azaspiro[4.5]dec-2-en-4-one;
benzyl 3-(benzo[d][1,3]dioxol-5-yl)-4-oxo-2-(pyridin-2-yl)-1-oxa-8-azaspiro[4.5]dec-2-ene-8-carboxylate;
4-(3-(benzo[d][1,3]dioxol-5-yl)-4-oxo-2-(pyridin-2-yl)-1-oxa-8-azaspiro[4.5]dec-2-enecarbonyl)benzonitrile;
3-(benzo[d][1,3]dioxol-5-yl)-2-(pyridin-2-yl)-8-tosyl-1-oxa-8-azaspiro[4.5]dec-2-en-4-one;
3-(benzo[d][1,3]dioxol-5-yl)-N-benzyl-4-oxo-2-(pyridin-2-yl)-1-oxa-8-azaspiro[4.5]dec-2-ene-8-carboxamide;
tert-butyl 2-(benzo[d][1,3]dioxol-5-yl)-4-oxo-3-(pyridin-2-yl)-1-oxa-8-azaspiro[4.5]dec-2-ene-8-carboxylate;
2-(benzo[d][1,3]dioxol-5-yl)-3-(pyri din-2-yl)-8-tosyl-1-oxa-8-azaspiro[4.5]dec-2-en-4-one;
2-(benzo[d][1,3]dioxol-5-yl)-8-(4-chlorophenylsulfonyl)-3-(pyridin-2-yl)-1-oxa-8-azaspiro[4.5]dec-2-en-4-one;
2-(benzo[d][1,3]dioxol-5-yl)-8-(3,4-dichlorophenylsulfonyl)-3-(pyridin-2-yl)-1-oxa-8-azaspiro[4.5]dec-2-en-4-one;
4-(2-(benzo[d][1,3]dioxol-5-yl)-4-oxo-3-(pyridin-2-yl)-1-oxa-8-azaspiro[4.5]dec-2-en-8-ylsulfonyl)benzoic acid;
4-(2-(benzo[d][1,3]dioxol-5-yl)-4-oxo-3-(pyridin-2-yl)-1-oxa-8-azaspiro[4.5]dec-2-en-8-ylsulfonyl)benzonitrile;
3-(benzo[d][1,3]dioxol-5-yl)-8-(4-chlorophenylsulfonyl)-2-(pyridin-2-yl)-1-oxa-8-azaspiro[4.5]dec-2-en-4-one;
3-(benzo[d][1,3]dioxol-5-yl)-8-(3,4-dichlorophenylsulfonyl)-2-(pyridin-2-yl)-1-oxa-8-azaspiro[4.5]dec-2-en-4-one;
4-(3-(benzo[d][1,3]dioxol-5-yl)-4-oxo-2-(pyridin-2-yl)-1-oxa-8-azaspiro[4.5]dec-2-en-8-ylsulfonyl)benzoic acid;
4-(3-(benzo[d][1,3]dioxol-5-yl)-4-oxo-2-(pyridin-2-yl)-1-oxa-8-azaspiro[4.5]dec-2-en-8-ylsulfonyl)benzonitrile;
2-([1,2,4]triazolo[1,5-a]pyridin-6-yl)-3-m-tolyl-1-oxaspiro[4.5]dec-2-ene-4,8-dione;
10-[1,2,4]Triazolo[1,5-a]pyridin-6-yl-11-(6-methylpyridine-2-yl)-1,4,9-trioxa-dispiro[4.2.4.2]tetradec-10-en-12-one;
10-(quinoxalin-6-yl)-11-(3-methylphenyl)-1,4,9-trioxa-dispiro[4.2.4.2]tetradec-10-en-12-one;
2-(quinoxalin-6-yl)-3-m-tolyl-1-oxaspiro[4.5]dec-2-ene-4,8-dione;
10-(quinoxalin-6-yl)-11-(3-chlorophenyl)-1,4,9-trioxa-dispiro[4.2.4.2]tetradec-10-en-12-one;
10-[1,2,4]Triazolo[1,5-a]pyridin-6-yl-11-(3-m-tolyl)-1,4,9-trioxa-dispiro[4.2.4.2]tetradec-10-en-12-one;
2-([1,2,4]triazolo[1,5-a]pyridin-6-yl)-8-hydroxy-3-m-tolyl-1-oxaspiro[4.5]dec-2-en-4-one;
3-(3-chlorophenyl)-2-(quinoxalin-6-yl)-1-oxaspiro[4.5]dec-2-ene-4,8-dione;
3-(3-chlorophenyl)-8-hydroxy-2-(quinoxalin-6-yl)-1-oxaspiro[4.5]dec-2-en-4-one;
8-hydroxy-2-(quinoxalin-6-yl)-3-m-tolyl-1-oxaspiro[4.5]dec-2-en-4-one;
ethyl 2-(4-oxo-2-(quinoxalin-6-yl)-3-m-tolyl-1-oxaspiro[4.5]dec-2-en-8-ylidene)acetate;
2-(4-oxo-2-(quinoxalin-6-yl)-3-m-tolyl-1-oxaspiro[4.5]dec-2-en-8-ylidene)acetic acid;
2-(4-oxo-2-(quinoxalin-6-yl)-3-m-tolyl-1-oxaspiro[4.5]dec-2-en-8-yl)acetic acid;
N-(2-morpholinoethyl)-2-(4-oxo-2-(quinoxalin-6-yl)-3-m-tolyl-1-oxaspiro[4.5]dec-2-en-8-yl)acetamide;
N-(2-(dimethylamino)ethyl)-2-(4-oxo-2-(quinoxalin-6-yl)-3-m-tolyl-1-oxaspiro[4.5]dec-2-en-8-yl)acetamide;
ethyl 2-(3-(3-chlorophenyl)-4-oxo-2-(quinoxalin-6-yl)-1-oxaspiro[4.5]dec-2-en-8-ylidene)acetate;
2-(3-(3-chlorophenyl)-4-oxo-2-(quinoxalin-6-yl)-1-oxaspiro[4.5]dec-2-en-8-yl)acetic acid;
2-([1,2,4]triazolo[1,5-a]pyridin-6-yl)-8-azido-3-m-tolyl-1-oxaspiro[4.5]dec-2-en-4-one;
2-([1,2,4]triazolo[1,5-a]pyridin-6-yl)-8-amino-3-m-tolyl-1-oxaspiro[4.5]dec-2-en-4-one;
N-(2-([1,2,4]triazolo[1,5-a]pyridin-6-yl)-4-oxo-3-m-tolyl-1-oxaspiro[4.5]dec-2-en-8-yl)methanesulfonamide;
N-methyl-2-(4-oxo-2-(quinoxalin-6-yl)-3-m-tolyl-1-oxaspiro[4.5]dec-2-en-8-yl)acetamide;
N-(2-methoxyethyl)-2-(4-oxo-2-(quinoxalin-6-yl)-3-m-tolyl-1-oxaspiro[4.5]dec-2-en-8-yl)acetamide;
2-(3-(3-chlorophenyl)-4-oxo-2-(quinoxalin-6-yl)-1-oxaspiro[4.5]dec-2-en-8-yl)-N-(2-morpholinoethyl)acetamide;
N-(2-([1,2,4]triazolo[1,5-a]pyridin-6-yl)-4-oxo-3-m-tolyl-1-oxaspiro[4.5]dec-2-en-8-yl)acetamide;
1-(2-([1,2,4]triazolo[1,5-a]pyridin-6-yl)-4-oxo-3-m-tolyl-1-oxaspiro[4.5]dec-2-en-8-yl)urea;
8-aminosulfonylamino-2-([1,2,4]triazolo[1,5-a]pyridin-6-yl)-3-m-tolyl-1-oxaspiro[4.5]dec-2-en-4-one;
5-([1,2,4]triazolo[1,5-a]pyridin-6-yl)-2,2-dimethyl-4-m-tolylfuran-3(2H)-one;
2-([1,2,4]triazolo[1,5-a]pyridin-6-yl)-8-hydroxy-3-(6-methylpyridin-2-yl)-1-oxaspiro[4.5]dec-2-en-4-one;
2-(4-oxo-2-(quinoxalin-6-yl)-3-m-tolyl-1-oxaspiro[4.5]dec-2-en-8-yl)acetamide;
ethyl 2-(2-([1,2,4]triazolo[1,5-a]pyridin-6-yl)-4-oxo-3-m-tolyl-1-oxaspiro[4.5]dec-2-en-8-ylidene)acetate;
2-(2-([1,2,4]triazolo[1,5-a]pyridin-6-yl)-4-oxo-3-m-tolyl-1-oxaspiro[4.5]dec-2-en-8-ylamino)-2-oxoethyl acetate;
methyl 3-(2-([1,2,4]triazolo[1,5-a]pyridin-6-yl)-4-oxo-3-m-tolyl-1-oxaspiro[4.5]dec-2-en-8-ylamino)-3-oxopropanoate;
dimethyl 3,3′-(2-([1,2,4]triazolo[1,5-a]pyridin-6-yl)-4-oxo-3-m-tolyl-1-oxaspiro[4.5]dec-2-en-8-ylazanediyl)bis(3-oxopropanoate);
3-(2-([1,2,4]triazolo[1,5-a]pyridin-6-yl)-4-oxo-3-m-tolyl-1-oxaspiro[4.5]dec-2-en-8-ylamino)-3-oxopropanoic acid;
N-hydroxy-2-(4-oxo-2-(quinoxalin-6-yl)-3-m-tolyl-1-oxaspiro[4.5]dec-2-en-8-yl)acetamide;
ethyl 2-(2-([1,2,4]triazolo[1,5-a]pyridin-6-yl)-4-oxo-3-m-tolyl-1-oxaspiro[4.5]dec-2-en-8-yl)acetate (isomer A);
ethyl 2-(2-([1,2,4]triazolo[1,5-a]pyridin-6-yl)-4-oxo-3-m-tolyl-1-oxaspiro[4.5]dec-2-en-8-yl)acetate (isomer B);
2-(2-([1,2,4]triazolo[1,5-a]pyridin-6-yl)-4-oxo-3-m-tolyl-1-oxaspiro[4.5]dec-2-en-8-ylidene)acetamide;
2-(2-([1,2,4]triazolo[1,5-a]pyridin-6-yl)-4-oxo-3-m-tolyl-1-oxaspiro[4.5]dec-2-en-8-ylidene)-N-(pyridin-3-ylmethyl)acetamide;
2-(2-([1,2,4]triazolo[1,5-a]pyridin-6-yl)-4-oxo-3-m-tolyl-1-oxaspiro[4.5]dec-2-en-8-ylidene)-N-(3-(2-oxopyrrolidin-1-yl)propyl)acetamide;
11-(3-chloro-4-fluoro-phenyl)-10-[1,2,4]triazolo[1,5-a]pyridin-6-yl-1,4,9-trioxa-dispiro[4.2.4.211tetradec-10-en-12-one;
3-(3-chloro-4-fluoro-phenyl)-2-[1,2,4]triazolo[1,5-a]pyridin-6-yl-1-oxa-spiro[4.5]dec-2-ene-4,8-dione;
ethyl 2-(2-([1,2,4]triazolo[1,5-a]pyridin-6-yl)-3-(3-chloro-4-fluorophenyl)-4-oxo-1-oxaspiro[4.5]dec-2-en-8-ylidene)acetate;
2-([1,2,4]triazolo[1,5-a]pyridin-6-yl)-8-(1,3-dioxolan-2-yl)-3-m-tolyl-1-oxaspiro[4,5]dec-2-en-4-one;
5-([1,2,4]triazolo[1,5-a]pyridin-6-yl)-4-(3-chlorophenyl)-2,2-dimethylfuran-3(2H)-one;
5-([1,2,4]triazolo[1,5-a]pyridin-6-yl)-4-(3-chloro-4-fluorophenyl)-2,2-dimethylfuran-3(2H)-one;
2-([1,2,4]triazolo[1,5-a]pyridin-6-yl)-8-((pyridin-3-ylamino ethyl)-3-m-tolyl-1-oxaspiro[4.5]dec-2-en-4-one;
2-([1,2,4]triazolo[1,5-a]pyridin-6-yl)-8-(1,3-dioxolan-2-yl)-3-(4-fluoro-3-methylphenyl)-1-oxaspiro[4.5]dec-2-en-4-one;
2-([1,2,4]triazolo[1,5-a]pyridin-6-yl)-3-(4-fluoro-3-methylphenyl)-4-oxo-1-oxaspiro[4.5]dec-2-ene-8-carboxylic acid; or
2-([1,2,4]triazolo[1,5-a]pyridin-6-yl)-3-(4-fluoro-3-methylphenyl)-4-oxo-1-oxaspiro[4.5]dec-2-ene-8-carboxamide.

77. A pharmaceutical composition comprising a compound of claim 56, and a pharmaceutically acceptable carrier.

78. A method of inhibiting the TGFβ signaling pathway in a subject, inhibiting the TGFβ type receptor in a cell, reducing the accumulation of excess extracellular matrix induced by TGFβ in a subject, of treating or preventing a fibrotic condition in a subject, treating restinosis, of treating or preventing vascular disease, of treating or preventing hypertension in a subject, of inhibiting growth or metastasis of tumor cells or cancer in a subject, or of treating a disease or disorder mediated by an overexpression of TGFβ in a subject, comprising administering to the subject in need thereof an effective amount of a compound of any of claim 56.

79. The method of claim 78, wherein the fibrotic condition is selected from the group consisting of scleroderma, lupus nephritis, connective tissue disease, wound healing, surgical scarring, spinal cord injury, CNS scarring, acute lung injury, idiopathic pulmonary fibrosis, radiation-induced pulmonary fibrosis, chronic obstructive pulmonary disease, adult respiratory distress syndrome, acute lung injury, drug-induced lung injury, glomerulonephritis, diabetic nephropathy, hypertension-induced nephropathy, alimentary track or gastrointestinal fibrosis, renal fibrosis, hepatic or biliary fibrosis, liver cirrhosis, primary biliary cirrhosis, fatty liver disease, primary sclerosing cholangitis, restenosis, radiation-induced fibrosis, chemotherapy-induced fibrosis, cardiac fibrosis, opthalmic scarring, fibrosclerosis, a fibrotic cancer, a fibroid, fibroma, a fibroadenoma, a fibrosarcoma, transplant arteriopathy, mesothelioma, and keloid or wherein the restenosis is coronary restenosis, peripheral restenosis, or carotid restenosis or wherein the vascular disease is intimal thickening, vascular remodeling, or organ transplant-related vascular disease or wherein the hypertension is primary or secondary hypertension, systolic hypertension, pulmonary hypertension, or hypertension-induced vascular remodeling.

80. A method of claim 78 wherein the disease or disorder is mediated by an overexpression of TGFβ in a subject, or such as where the disease or disorder is a carcinoma, and wherein the carcinoma is for example a carcinoma of the lung, breast, liver, biliary tract, gastrointestinal tract, head and neck, pancreas, prostate, cervix, multiple myeloma, melanoma, glioma, or glioblastomas; or
wherein, the disease or disorder is selected from the group consisting of demyelination of neurons in multiple sclerosis, Alzheimer's disease, cerebral angiopathy, squamous cell carcinomas, multiple myeloma, melanoma, glioma, glioblastomas, leukemia, sarcomas, leiomyomas, mesothelioma, and carcinomas of the lung, breast, ovary, cervix, liver, biliary tract, gastrointestinal tract, pancreas, prostate, head, and neck, comprising administering to the subject in need thereof an effective amount of a compound of claim 56.
